SEQUENCE LISTING (47 sequences)

SEQ ID NO: 1 - DNA, 2235 bp, Homo sapiens
atggagcaggatcccaagccgccccgtctgcggctctgggccctgatcccctggcttcccaggaagcagcggcccaggatcagccagacctctctgcctgtccctggccctggctctggcccccagcgggactcggatgagggcgtcctcaaggagatctccatcacgcaccacgtcaaggctggctctgagaaggctgatccatcccatttcgagctcctcaaggttctgggccagggatcctttggcaaagtcttcctggtgcggaaagtcacccggcctgacagtgggcacctgtatgctatgaaggtgctgaagaaggcaacgctgaaagtacgtgaccgcgtccggaccaagatggagagagacatcctggctgatgtaaatcacccattcgtggtgaagctgcactatgccttccagaccgagggcaagctctatctcattctggacttcctgcgtggtggggacctcttcacccggctctcaaaagaggtgatgttcacggaggaggatgtgaagttttacctggccgagctggctctgggcctggatcacctgcacagcctgggtatcatttacagagacctcaagcctgagaacatccttctggatgaggagggccacatcaaactcactgactttggcctgagcaaagaggccattgaccacgagaagaaggcctattctttctgcgggacagtggagtacatggcccctgaggtcgtcaaccgccagggccactcccatagtgcggactggtggtcctatggggtgttgatgtttgagatgctgacgggctccctgcccttccaggggaaggaccggaaggagaccatgacactgattctgaaggcgaagctaggcatgccccagtttctgagcactgaagcccagagcctcttgcgggccctgttcaagcggaatcctgccaaccggctcggctccggccctgatggggcagaggaaatcaagcggcatgtcttctactccaccattgactggaataagctataccgtcgtgagatcaagccacccttcaagccagcagtggctcagcctgatgacaccttctactttgacaccgagttcacgtcccgcacacccaaggattccccaggcatcccccccagcgctggggcccatcagctgttccggggcttcagcttcgtggccaccggcctgatggaagacgacggcaagcctcgtgccccgcaggcacccctgcactcggtggtacagcaactccatgggaagaacctggtttttagtgacggctacgtggtaaaggagacaattggtgtgggctcctactctgagtgcaagcgctgtgtccacaaggccaccaacatggagtatgctgtcaaggtcattgataagagcaagcgggatccttcagaagagattgagattcttctgcggtatggccagcaccccaacatcatcactctgaaagatgtgtatgatgatggcaaacacgtgtacctggtgacagagctgatgcggggtggggagctgctggacaagatcctgcggcagaagttcttctcagagcgggaggccagctttgtcctgcacaccattggcaaaactgtggagtatctgcactcacagggggttgtgcacagggacctgaagcccagcaacatcctgtatgtggacgagtccgggaatcccgagtgcctgcgcatctgtgactttggttttgccaaacagctgcgggctgagaatgggctcctcatgacaccttgctacacagccaactttgtggcgcctgaggtgctgaagcgccagggctacgatgaaggctgcgacatctggagcctgggcattctgctgtacaccatgctggcaggatatactccatttgccaacggtcccagtgacacaccagaggaaatcctaacccggatcggcagtgggaagtttaccctcagtgggggaaattggaacacagtttcagagacagccaaggacctggtgtccaagatgctacacgtggatccccaccagcgcctcacagctaagcaggttctgcagcatccatgggtcacccagaaagacaagcttccccaaagccagctgtcccaccaggacctacagcttgtgaagggagccatggctgccacgtactccgcactcaacagctccaagcccaccccccagctgaagcccatcgagtcatccatcctggcccagcggcgagtgaggaagttgccatccaccaccctgtga

SEQ ID NO: 2 - protein, 744 aa, Homo sapiens
MEQDPKPPRLRLWALIPWLPRKQRPRISQTSLPVPGPGSGPQRDSDEGVLKEISITHHVKAGSEKADPSHFELLKVLGQGSFGKVFLVRKVTRPDSGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHSHSADWWSYGVLMFEMLTGSLPFQGKDRKETMTLILKAKLGMPQFLSTEAQSLLRALFKRNPANRLGSGPDGAEEIKRHVFYSTIDWNKLYRREIKPPFKPAVAQPDDTFYFDTEFTSRTPKDSPGIPPSAGAHQLFRGFSFVATGLMEDDGKPRAPQAPLHSVVQQLHGKNLVFSDGYVVKETIGVGSYSECKRCVHKATNMEYAVKVIDKSKRDPSEEIEILLRYGQHPNIITLKDVYDDGKHVYLVTELMRGGELLDKILRQKFFSEREASFVLHTIGKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTPEEILTRIGSGKFTLSGGNWNTVSETAKDLVSKMLHVDPHQRLTAKQVLQHPWVTQKDKLPQSQLSHQDLQLVKGAMAATYSALNSSKPTPQLKPIESSILAQRRVRKLPSTTL

SEQ ID NO: 3 - DNA, 2253 bp, Homo sapiens
atgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatag

SEQ ID NO: 4 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 5 - protein, 20 aa, Artificial Sequence
  Description of Artificial Sequence Synthetic peptide
  MOD_RES (10)..(10): Met-oxide
  MOD_RES (11)..(11): PhosphoSer
LQTTDNLLPMSPEEFDEVSR

SEQ ID NO: 6 - RNA, 19 nt, Unknown
  Description of Unknown non-targeting control pool sequence
ugguuuacaugucgacuaa

SEQ ID NO: 7 - RNA, 19 nt, Unknown
  Description of Unknown non-targeting control pool sequence
ugguuuacauguuguguga

SEQ ID NO: 8 - RNA, 19 nt, Unknown
  Description of Unknown non-targeting control pool sequence
ugguuuacauguuuucuga

SEQ ID NO: 9 - RNA, 19 nt, Unknown
  Description of Unknown non-targeting control pool sequence
ugguuuacauguuuuccua

SEQ ID NO: 10 - RNA, 19 nt, Homo sapiens
gugggcaccuguaugcuau

SEQ ID NO: 11 - RNA, 19 nt, Homo sapiens
gauaagagcaagcgggauc

SEQ ID NO: 12 - RNA, 19 nt, Homo sapiens
gaaaguacgugaccgcguc

SEQ ID NO: 13 - RNA, 19 nt, Homo sapiens
gaacacaguuucagagaca

SEQ ID NO: 14 - protein, 19 aa, Artificial Sequence
  Description of Artificial Sequence Synthetic peptide
  MOD_RES (5)..(5): PhosphoThr
RRAETFGGFDSHQMNASKG

SEQ ID NO: 15 - protein, 10 aa, Artificial Sequence
  Description of Artificial Sequence Synthetic peptide
  MOD_RES (4)..(4): PhosphoThr
RLNTSDFQKL

SEQ ID NO: 16 - protein, 11 aa, Artificial Sequence
  Description of Artificial Sequence Synthetic peptide
  MOD_RES (4)..(4): PhosphoThr
RSKTEEDILRA

SEQ ID NO: 17 - protein, 17 aa, Artificial Sequence
  Description of Artificial Sequence Synthetic peptide
  MOD_RES (4)..(4): PhosphoThr
RASTLLRDEELEEIKKE

SEQ ID NO: 18 - protein, 19 aa, Artificial Sequence
  Description of Artificial Sequence Synthetic peptide
  MOD_RES (4)..(4): PhosphoSer
RRQSSGSATNVASTPDNRG

SEQ ID NO: 19 - protein, 22 aa, Artificial Sequence
  Description of Artificial Sequence Synthetic peptide
  MOD_RES (4)..(4): PhosphoSer
RRKSQVNGEAGSYEMTNQHVKQ

SEQ ID NO: 20 - protein, 19 aa, Artificial Sequence
  Description of Artificial Sequence Synthetic peptide
  MOD_RES (6)..(6): PhosphoSer
RLRRPSVNGEPGSVPPPRA

SEQ ID NO: 21 - protein, 19 aa, Artificial Sequence
  Description of Artificial Sequence Synthetic peptide
  MOD_RES (4)..(4): PhosphoSer
RTGSESSQTGTSTTSSRSN

SEQ ID NO: 22 - protein, 19 aa, Artificial Sequence
  Description of Artificial Sequence Synthetic peptide
  MOD_RES (5)..(5): PhosphoSer
RRAPSVANVGSHCDLSLKI

SEQ ID NO: 23 - protein, 24 aa, Artificial Sequence
  Description of Artificial Sequence Synthetic peptide
  MOD_RES (5)..(5): PhosphoSer
RRHNSSDGFDSAIGRPNGGNFGRK

SEQ ID NO: 24 - protein, 23 aa, Artificial Sequence
  Description of Artificial Sequence Synthetic peptide
  MOD_RES (4)..(4): PhosphoSer
RHNSSDGFDSAIGRPNGGNFGRK

SEQ ID NO: 25 - protein, 13 aa, Artificial Sequence
  Description of Artificial Sequence Synthetic peptide
  MOD_RES (4)..(4): PhosphoThr
RSATLETKPESKY

SEQ ID NO: 26 - protein, 19 aa, Artificial Sequence
  Description of Artificial Sequence Synthetic peptide
  MOD_RES (4)..(4): PhosphoThr
RLTTLALNGNRLTRAVLRD

SEQ ID NO: 27 - protein, 13 aa, Artificial Sequence
  Description of Artificial Sequence Synthetic peptide
  MOD_RES (4)..(4): PhosphoThr
RKRTEEPDRDERL

SEQ ID NO: 28 - protein, 23 aa, Artificial Sequence
  Description of Artificial Sequence Synthetic peptide
  MOD_RES (4)..(4): PhosphoThr
RRNTTQNTGYSSGTQNANYPVRA

SEQ ID NO: 29 - protein, 16 aa, Artificial Sequence
  Description of Artificial Sequence Synthetic peptide
  MOD_RES (4)..(4): PhosphoSer
RTASGSSVTSLDGTRS

SEQ ID NO: 30 - protein, 19 aa, Artificial Sequence
  Description of Artificial Sequence Synthetic peptide
  MOD_RES (4)..(4): PhosphoSer
RTHSTSSSLGSGESPFSRS

SEQ ID NO: 31 - protein, 20 aa, Artificial Sequence
  Description of Artificial Sequence Synthetic peptide
  MOD_RES (5)..(5): PhosphoSer
RRLKSEDELRPEVDEHTQKT

SEQ ID NO: 32 - protein, 19 aa, Artificial Sequence
  Description of Artificial Sequence Synthetic peptide
  MOD_RES (4)..(4): PhosphoSer
RLKSEDELRPEVDEHTQKT

SEQ ID NO: 33 - protein, 15 aa, Artificial Sequence
  Description of Artificial Sequence Synthetic peptide
  MOD_RES (6)..(6): PhosphoSer
KRRRLSSLRASTSKS

SEQ ID NO: 34 - protein, 13 aa, Artificial Sequence
  Description of Artificial Sequence Synthetic peptide
  MOD_RES (4)..(5): PhosphoSer
RRLSSLRASTSKS

SEQ ID NO: 35 - protein, 11 aa, Artificial Sequence
  Description of Artificial Sequence Synthetic peptide
  MOD_RES (6)..(6): PhosphoSer
RERLPSVDLKE

SEQ ID NO: 36 - protein, 13 aa, Artificial Sequence
  Description of Artificial Sequence Synthetic peptide
  MOD_RES (4)..(4): PhosphoSer
RENSTVDFSKVEG

SEQ ID NO: 37 - protein, 15 aa, Artificial Sequence
  Description of Artificial Sequence Synthetic peptide
  MOD_RES (4)..(4): PhosphoSer
RRSSTSSEPTPTVKT

SEQ ID NO: 38 - protein, 17 aa, Artificial Sequence
  Description of Artificial Sequence Synthetic peptide
  MOD_RES (4)..(4): PhosphoSer
RRASISEPSDTDPEPRT

SEQ ID NO: 39 - protein, 17 aa, Artificial Sequence
  Description of Artificial Sequence Synthetic peptide
  MOD_RES (4)..(4): PhosphoSer
RASSRVSGSFPEDSSKE

SEQ ID NO: 40 - protein, 15 aa, Artificial Sequence
  Description of Artificial Sequence Synthetic peptide
  MOD_RES (4)..(4): PhosphoSer
RRPSTEDTHEVDSKA

SEQ ID NO: 41 - protein, 4 aa, Artificial Sequence
  Description of Artificial Sequence Synthetic peptide
  MOD_RES (2)..(3): Any amino acid
  MOD_RES (4)..(4): PhosphoSer or PhosphoThr
RXXX

SEQ ID NO: 42 - protein, 4 aa, Artificial Sequence
  Description of Artificial Sequence Synthetic peptide
  MOD_RES (2)..(3): Any amino acid
  MOD_RES (4)..(4): Ser or Thr
RXXX

SEQ ID NO: 43 - protein, 4 aa, Artificial Sequence
  Description of Artificial Sequence Synthetic peptide
  MOD_RES (4)..(4): PhosphoSer
LPMS

SEQ ID NO: 44 - protein, 4 aa, Artificial Sequence
  Description of Artificial Sequence Synthetic peptide
  MOD_RES (4)..(4): PhosphoSer
YLRS

SEQ ID NO: 45 - protein, 4 aa, Artificial Sequence
  Description of Artificial Sequence Synthetic peptide
  MOD_RES (4)..(4): PhosphoSer
MQPS

SEQ ID NO: 46 - protein, 4 aa, Artificial Sequence
  Description of Artificial Sequence Synthetic peptide
  MOD_RES (4)..(4): PhosphoSer
PNRS

SEQ ID NO: 47 - protein, 19 aa, Homo sapiens
KAYSFCGTVEYMAPEVVNR

(19) United States
(12) Patent Application Publication
(10) Pub. No.: US 2021/0123061 A1
(43) Pub. Date: Apr. 29, 2021

(54) METHODS AND COMPOSITIONS FOR TREATING INFLAMMATORY DISEASE OR DISORDER

(71) Applicant: THE BRIGHAM AND WOMEN'S HOSPITAL, INC., Boston, MA (US)

(72) Inventors: Keishi NIHIRA, Boston, MA (US); Masanori AIKAWA, Chestnut Hill, MA (US); Sasha SINGH, Boston, MA (US); Arda HALU, Brookline, MA (US); Takaharu ASANO, Brookline, MA (US)

(73) Assignee: THE BRIGHAM AND WOMEN'S HOSPITAL, INC., Boston, MA (US)

(21) Appl. No.: 17/052,656

(22) PCT Filed: May 3, 2019
(86) PCT No.: PCT/US2019/030546

§ 371 Date: Nov. 3, 2020

Related U.S. Application Data
(60) Provisional application No. 62/666,787, filed on May 4, 2018.

(51) Int. Cl. C12N 15/113; C07K 16/40; A61K 31/519; A61K 31/336; A61K 31/395; A61K 31/167; A61K 31/216; A61P 37/06

(52) U.S. Cl. CPC C12N 15/1137; C07K 16/40; A61K 31/519; A61K 31/336; A61K 31/395; C07K 2317/24; A61K 31/216; A61P 37/06; C12N 2310/141; C12N 2310/14; C07K 2317/76; A61K 31/167

(57) ABSTRACT

Described herein are methods and compositions for treating inflammatory disease. Aspects of the invention relates to administering to a subject an agent that inhibits RSK1. Another aspect of the invention relates to administering the STAT1 phosphorylation.

### CROSS REFERENCE TO RELATED APPLICATION

This application is a 35 U.S.C. § 371 National Phase Entry Application of International Application No. PCT/US2019/030546 filed May 3, 2019, which designates the U.S. and which claims the benefit under 35 U.S.C. § 119(e) of U.S. Provisional Application No. 62/666,787, filed on May 4, 2018, the contents of which is incorporated herein by reference in their entireties.

### SEQUENCE LISTING

The instant application contains a Sequence Listing which has been submitted electronically in ASCII format and is hereby incorporated by reference in its entirety. Said ASCII copy, created on Nov. 3, 2020, is named 043214-092230WOPT_SL.txt and is 32,582 bytes in size.

### FIELD OF THE INVENTION

The field of the invention relates to the treatment of inflammatory disease or disorder.

### BACKGROUND

Pro-inflammatory activation of macrophages plays a critical role in the pathogenesis of multiple human diseases. Macrophages are a heterogeneous population. Transcriptomics studies have demonstrated that stimuli, such as interferon-γ (IFN-γ) and LPS, skew macrophages towards subsets of pro-inflammatory phenotypes warranting their polarization state to be classified according to their stimulant conditions, for example, M(IFN-γ) and M(LPS) macrophages. Similarly, anti-inflammatory stimuli such as interleukin-4 (IL-4) and IL-13 shift their phenotype toward the subset of alternative activation, e.g., M (IL-4) and M(IL-13), to resolve inflammatory responses (5, 7-10). Nonetheless, M(IFN-γ) and M(LPS) macrophages commonly express high levels of pro-inflammatory chemokines such as CCL2/MCP-1 to recruit immune cells to inflamed sites, permitting either stimulant to be used as an inducer of general pro-inflammatory signaling events. Although emerging evidence suggests a theory that macrophage heterogeneity is multidimensional rather than a conventional M1/M2 polarization paradigm, the use of an in vitro model of each phenotype such as M(IFN-γ), where specific cause-effect relationships are known, helps identify new molecular mechanisms.

Intracellular signaling mechanisms, including the Janus kinase-signal transducer and activator of transcription (JAK-STAT) pathway, mediate IFN-γ-triggered pro-inflammatory cellular responses. IFN-γ stimulation leads to STAT1 phosphorylation at Tyr701 (phospho-STAT1-Tyr701) by JAK1 and JAK2 in the cytoplasm, promoting nuclear translocation of phospho-STAT1-Tyr701. In the nucleus, phospho-STAT1-Tyr701 then undergoes phosphorylation at Ser727 (phospho-STAT1-Tyr701/Ser727), leading to transactivation of STAT1-target genes to produce chemokines in macrophages. These lines of evidence support the notion that nuclear-targeting molecules regulate pro-inflammatory activation of macrophages. However, an understanding of mechanisms and roles for nuclear translocation of such potential regulators in pro-inflammatory activation of macrophages remains limited.

### SUMMARY

The invention described herein is based partly on the finding that IFN-γ stimulation results in RSK1 phosphorylation at Ser380 via JAK signaling, causing its translocation into the nucleus of macrophages. Nuclear RSK1 phosphorylates STAT1 at Ser727, in the nuclei of macrophages, activating the macrophage. Importantly, inhibition of RSK1 hinders IFN-γ-induced secretion of pro-inflammatory chemokines in human primary macrophages. Accordingly, one aspect described herein provides a method of treating an inflammatory disease or disorder comprising administering to a subject in need thereof an effective amount of an agent that inhibits Ribosomal S6 Kinase-1 (RSK1).

In one embodiment of any aspect, inhibition of RSK1 is the inhibition of RSK1 phosphorylation. In one embodiment, the RSK1 phosphorylation is at Serine 380.

In one embodiment of any aspect, inhibition of RSK1 is the inhibition of RSK1 nuclear translocation.

In one embodiment of any aspect, inhibition of RSK1 is the inhibition of RSK1 kinase activity. In one embodiment of any aspect, inhibition of RSK1 kinase activity inhibits the phosphorylation of Signal transducer and activator of transcription 1 (STAT1). In one embodiment of any aspect, the phosphorylation of STAT1 is at Serine 727.

In one embodiment of any aspect, inhibition of RSK1 inhibits the inflammatory response.

In one embodiment of any aspect, the agent that inhibits RSK1 is selected from the group consisting of a small molecule, an antibody, a peptide, a genome editing system, an antisense oligonucleotide, and an RNAi. In one embodiment of any aspect, the RNAi is a microRNA, an siRNA, or a shRNA. In one embodiment of any aspect, the antibody is a humanized antibody.

In one embodiment of any aspect, the small molecule is MK-1775, Manumycin-a, Cerulenin, Tanespimycin, salermide, or tosedostat.

In one embodiment of any aspect, inhibiting RSK1 is inhibiting the expression level and/or activity of RSK1. In one embodiment of any aspect, the expression level and/or activity of RSK1 is inhibited by at least 50%, at least 60%, at least 70%, at least 80%, at least 90%, or more as compared to an appropriate control.

In one embodiment of any aspect, wherein inhibition of RSK1 suppresses IFN-γ-induced chemokines in primary macrophages.

In one embodiment of any aspect, the IFN-γ-induced chemokines are suppressed by at least 50%, at least 60%, at least 70%, at least 80%, at least 90%, or more as compared to an appropriate control.

Another aspect described herein provides a method of treating an inflammatory disease or disorder, the method comprising administering to a subject in need thereof an effective amount of an agent that inhibits Signal transducer and activator of transcription 1 (STAT1) phosphorylation.

In one embodiment of any aspect, STAT1 phosphorylation is at Serine 727.

In one embodiment of any aspect, inhibition of STAT1 phosphorylation inhibits the inflammatory response.

In one embodiment of any aspect, the agent that inhibits STAT1 phosphorylation is selected from the group consisting of a small molecule, an antibody, a peptide, a genome editing system, an antisense oligonucleotide, and an RNAi. In one embodiment of any aspect, the RNAi is a microRNA, an siRNA, or a shRNA. In one embodiment of any aspect, the antibody is a humanized antibody.

In one embodiment of any aspect, the phosphorylation is inhibited by at least 50%, at least 60%, at least 70%, at least 80%, at least 90%, or more as compared to an appropriate control.

In one embodiment of any aspect, the method further comprises, prior to administration, diagnosing a subject with having an inflammatory disease or disorder.

In one embodiment of any aspect, the method further comprises, prior to administration, receiving results that identify a subject as having an inflammatory disease or disorder.

In one embodiment of any aspect, the method further comprises administering at least a second therapeutic for an inflammatory disease or disorder.

In one embodiment of any aspect, the subject has not been previously diagnosed with or identified as having an inflammatory disease or disorder. In one embodiment of any aspect, the subject has been previously diagnosed with or identified as having an inflammatory disease or disorder.

In one embodiment of any aspect, the inflammatory disease or disorder is selected from the group consisting of but is not limited to: macrophage activation syndrome, ulcerative colitis, type II diabetes, rheumatoid arthritis, juvenile idiopathic arthritis, Takayasu disease, aortic stenosis, Coffin-Lowry syndrome, pulmonary hypertension, Gaucher disease, systemic lupus erythematosus, Buerger disease, atherosclerosis, coronary artery disease, myocardial infarction, peripheral artery disease, vein graft disease, in-stent restenosis, arterioveneous fistula disease, arterial calcification, calcific aortic valve disease, Crohn's disease, vasculitis syndrome, scleroderma, rheumatic heart disease, acute lung injury, chronic obstructive pulmonary disease, acute kidney injury, stroke, neuroinflammation, and fatty liver.

Another aspect provided herein is a method of inhibiting macrophage activation, comprising administering to a subject in need thereof an effective amount of an agent that inhibits RSK1.

Another aspect provided herein is a method of inhibiting macrophage activation, comprising administering to a subject in need thereof an effective amount of an agent that inhibits STAT1 phosphorylation.

Another aspect provided herein is a composition comprising an agent that inhibits RSK1.

Another aspect provided herein is a composition comprising an agent that inhibits STAT1 phosphorylation.

In one embodiment of any aspect, the composition further comprises a pharmaceutically acceptable carrier.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with an inflammatory disease or disorder. The term “treating” includes reducing or alleviating at least one adverse effect or symptom of an inflammatory disease or disorder (e.g., skin rash, fatigue, joint pain, etc.). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., inflammatory disease or disorder) does not occur due to the actions of the methodology (such as, for example, administration of an agent that inhibits RSK1, or STAT1 phosphorylation, or a composition described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition described herein).

As used herein, the term “administering,” refers to the placement of a therapeutic (e.g., an agent that inhibits RSK1, or STAT1 phosphorylation) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., inflammatory disease or disorder. A subject can be male or female. A subject can be a child (e.g., less than 18 years of age), or an adult (e.g., greater than 18 years of age).

A subject can be one who has been previously diagnosed with or identified as suffering from or having a disease or disorder in need of treatment (e.g., inflammatory disease or disorder) or one or more complications related to such a disease or disorder (e.g., myocardial infarction, vein graft failure), and optionally, have already undergone treatment (e.g., statin therapy) for the disease or disorder or the one or more complications related to the disease or disorder. Alternatively, a subject can also be one who has not been previously diagnosed as having such disease or disorder (e.g., inflammatory disease or disorder) or related complications (e.g., myocardial infarction, vein graft failure). For example, a subject can be one who exhibits one or more risk factors for the disease or disorder or one or more complications related to the disease or disorder or a subject who does not exhibit risk factors. Risk factors for inflammatory disease or disorder include, but are not limited to, increased age, obesity, dyslipidemia, hypertension, diabetes, chronic kidney disease, diet of high saturated fats, reduced sex hormones (e.g., testosterone or estrogen), smoking, and having a sleep disorder (e.g., sleep apnea and narcolepsy).

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit RSK1, or STAT1 phosphorylation, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

Methods and compositions described herein require that the levels and/or activity of RSK1 are inhibited. As used herein, “Ribosomal Protein S6 A1 (RSK1)”, also known as HU-1, RSK, p90RSK, and MAPKAPK1A refers to kinase that has been implicated in controlling cell growth and differentiation. RSK1 kinase substrates include members of the MAPK signaling pathway. RSK1 sequences are known for a number of species, e.g., human RSK1 (NCBI Gene ID: 6195) polypeptide (e.g., NCBI Ref Seq NP_001006666.1) and mRNA (e.g., NCBI Ref Seq NM_001006665.1). RSK1 can refer to human RSK1, including naturally occurring variants, molecules, and alleles thereof. RSK1 refers to the mammalian RSK1 of, e.g., mouse, rat, rabbit, dog, cat, cow, horse, pig, and the like. The nucleic sequence of SEQ ID NO: 1 comprises a nucleic sequence which encodes RSK1.

Methods and compositions described herein require that the levels and/or activity of phosphorylated STAT1 are inhibited. As used herein, “signal transducer and activator of transcription 1 (STAT1),” also known as CANDF7; IMD31A; IMD31B; IMD31C; ISGF-3; and STAT91 refers to a protein that, in response to phosphorylation, form homo- or heterodimers that translocate to the cell nucleus where they act as a transcription activator. STAT1 sequences are known for a number of species, e.g., human STAT1 (NCBI Gene ID: 6772) polypeptide (e.g., NCBI Ref Seq NP_009330.1) and mRNA (e.g., NCBI Ref Seq NM_007315.3). STAT1 can refer to human STAT1, including naturally occurring variants, molecules, and alleles thereof. STAT1 refers to the mammalian STAT1 of, e.g., mouse, rat, rabbit, dog, cat, cow, horse, pig, and the like. The nucleic sequence of SEQ ID NO: 3 comprises a nucleic sequence which encodes STAT1.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an inflammatory disease or disorder, or a biological sample that has not been contacted with an agent or composition thereof disclosed herein).

As used herein, an “appropriate control” refers to an untreated, otherwise identical cell or population (e.g., a patient who was not administered an agent or composition thereof described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1H show the identification of RSK1 nuclear translocating in human primary macrophages in response to IFN-γ. (FIG. 1A) Proteomics workflow to identify nuclear translocating enzymes using tandem mass tagging (TMT) and LC-MS/MS. (FIG. 1B) Percent enrichment of nuclear proteins according to three public databases. (FIG. 1C) A more detailed distribution of protein compartment localization according to Uniprot.org. “Other localization” indicates that annotation of subcellular localization do not include the nucleus or nuclear organelles. “Nuclear shuttling” indicates that annotation of subcellular localization includes the nucleus or nuclear organelle, plus other intracellular organelles. (FIG. 1D) Reference normalized traces of STAT1 and RSK1 proteins over the IFN-γ stimulation period, compared to the average trace for the entire nuclear proteomics data. (FIG. 1E) Cell lysates of human PBMC-derived macrophages were subjected to immunoblot analysis with anti-RSK1, anti-RSK2, anti-RSK3, anti-RSK4, and anti-Tubulin. Recombinant RSK1, RSK2, RSK3, and RSK4 proteins were used as positive control for immunoblotting. Equal amount of recombinant proteins was confirmed by Coomassie blue staining. (FIG. 1F and FIG. 1G) Human PBMC-derived macrophages were stimulated with IFN-γ for 30 minutes. Cells were fixed and stained with anti-RSK1. Nuclei were stained with 4,6-diamidino-2-phenylindole (DAPI). (FIG. 1H) Human PBMC-derived macrophages were stimulated with IFN-γ. Nuclear lysates were subjected with immunoblotting with anti-RSK1, anti-STAT1, or anti-Lamin A/C, anti-Tubulin. Whole cell lysates (WCL) were used as control for blotting with anti-Tubulin.

FIG. 2 shows a network analysis links RSK1 with inflammatory diseases. Schematic showing the shared diseases that are significantly close (empirical p-value <0.05) in the interactome to the RSK1-, RSK2-, RSK3-, and RSK4-first neighbor modules. The average shortest distance of the same number of randomly selected genes to disease genes was calculated for N=250 realizations in order to compare the average shortest distance value to random expectation. Empirical p-values were calculated based on 100 degree-preserved randomizations of the first neighbor networks.

FIGS. 3A-3F show RSK1 is activated through JAK signaling and its inhibition suppresses STAT1 phosphorylation at Ser727 in human primary macrophages in response to IFN-γ. (FIG. 3A) PBMC-derived macrophages were pre-treated with DMSO or 10 μM a pan-JAK inhibitor, pyridone-6, for two hours and then stimulated with IFN-γ for indicated time under serum starvation. Cell lysates were subjected to immunoprecipitation with normal IgG or anti-RSK1 followed by immunoblot analysis using the indicated antibodies. WCL, whole cell lysate. N=3 donors. (FIG. 3B) Immunoblots of cell lysates from macrophages that were transfected with control siRNA or RSK1 siRNA followed by stimulation with IFN-γ for indicated time under serum starvation. N=2 donors. (FIG. 3C) Immunoblots of cell lysates from macrophages that were pre-treated with DMSO or BI-D1870 for two hours and subsequently left unstimulated or stimulated with IFN-γ for 1 hour under serum starvation. (FIG. 3D) Densitometric based quantification (ImageJ Software) of STAT1-pSer727 in panel (FIG. 3C) plus three additional donors. The phosphorylation levels in cells pretreated with DMSO and stimulated were defined as 1.0. Data are means±SE. N=6 donors. **P<0.01 indicate significance for phosphorylation levels compared to control (treatment with DMSO and IFN-γ) by Dunnett's comparison test. (FIG. 3E) Macrophages were treated with DMSO or BI-d1870 for two hours followed by IFN-γ stimulation under serum starvation. Cell lysates were subjected to immunoprecipitation with normal IgG or anti-STAT1-pSer727 followed by SYPRO ruby stain or immunoblot analysis with the indicated antibodies. Immunoprecipitants in the gel (red square) were in-gel digested for parallel reaction monitoring (PRM) mass spectrometry. (FIG. 3F) Quantification of PRM ions (MS/MS ions) of the indicated STAT1 peptide harboring a phosphorylation at Ser727 (and oxidized methionine, m), generated from the digest of panel (FIG. 3F).

FIGS. 4A-D show silencing RSK1 suppresses IFN-γ-induced chemokines in human primary macrophages. (FIG. 4A and FIG. 4B) Human PBMC-derived macrophages were treated with control siRNA or RSK1 siRNA followed by treatment with IFN-γ for the indicated time. Total RNA samples were subjected to real-time PCR analysis using the indicated probes and primers. GAPDH was used for normalization. (FIG. 4A) Representative results from one donor. Data are means±SD. (FIG. 4B) Quantification of the area-under-the-curve (AUC) plots of the real-time PCR data in panel (FIG. 4A). Data are means±SE. N=3 donors. *P<0.05 and **P<0.01 indicate significance for AUC of mRNA levels by paired Student's test. (FIG. 4C) Macrophages were treated with DMSO or BI-D1870 (1 or 10 μM) for two hours followed by stimulation with IFN-γ for eight hours. Total RNA samples were subjected to real-time PCR analysis using the indicated probes and primers. GAPDH was used for normalization. Data are means±SE. N=3-4 donors. *P<0.05 and **P<0.01 indicate significance for mRNA levels compared to control (treatment with DMSO and IFN-γ) by Dunnett's comparison test. (FIG. 4D) Macrophages were transfected with control siRNA or RSK1 siRNA followed by treatment with IFN-γ for 24 hours. Secreted chemokine levels were measured using ELISA. Data are means±SE. N=3 donors. *P<0.05 and **P<0.01 indicate significance for secretion of chemokines by paired Student's test.

FIGS. 5A-5D show RSK activity plays a key role for pro-inflammatory activation of macrophages in peritonitis model. (FIG. 5A) Model overview—mice were injected intraperitoneally with vehicle or 30 mg/kg BI-D1870. Twenty-four hours after the injection, 4% thioglycollate as well as vehicle or 30 mg/kg BI-D1870 were injected intraperitoneally in the mice. Forty-eight hours after the first injection, peritoneal cells were harvested. (FIG. 5B) Representative results of flow cytometry. Peritoneal cells were incubated with APC-Cy7-anti-CD45, FITC-anti-F4/80, APC-anti-CD11b, and PE-Cy7-anti-CD86 followed by flow-cytometry analysis. (FIG. 5C) Ratio of CD86-positive cells in peritoneal macrophages. Data are means±SE. N=10 mice. *P<0.05 indicates significance compared to control (vehicle) by unpaired Student's test. (FIG. 5D) Cell numbers of peritoneal CD86-positive macrophages. Data are means±SE. N=10 mice. *P<0.05 indicates significance by unpaired Student's test.

FIGS. 6A-6C show phospho-proteomics identified RSK substrates in human PBMC-derived macrophages. (FIG. 6A) Scheme of phospho-proteomics. Human PBMC-derived macrophages were treated with DMSO or BI-D1870 for two hours followed by IFN-γ stimulation for subsequent proteolysis and phospho-peptide enrichment using the anti-RXXpS/T antibody strategy. (FIG. 6B) Cell lysates were subjected to immunoblot analysis with anti-RPS6-pSer235/236, anti-RPS6, anti-PRAS40-pThr246, anti-PRAS40, or anti-Tubulin. N=2 donors. (FIG. 6C) Schematic showing the shared diseases that are significantly close (empirical p-value <0.05) in the interactome to the RSK-substrates modules. The average shortest distance of the same number of randomly selected genes to disease genes was calculated for N=250 realizations in order to compare the average shortest distance value to random expectation. Empirical p-values were calculated based on 100 degree-preserved randomizations of the first neighbor networks.

FIGS. 7A-7B show screening for nuclear translocating proteins. (FIG. 7A) High-dimensional cluster analysis revealed early-increasing patterns and late-increasing patterns in the dataset. We selected 11 clusters (red traces) as an early-increasing pattern and 9 clusters (blue traces) as a late-increasing pattern. (FIG. 7B) A flow chart of screening for nuclear translocating enzymes. We selected RPS6KA1 (RSK1) as a candidate enzyme that translocates to the nucleus for pro-inflammatory activation in macrophages.

FIG. 8 shows the RSK enzyme family. This schematic representation of the RSK enzyme family is based on, e.g., Y. Romeo, X. Zhang, P. P. Roux, Regulation and function of the RSK family of protein kinases. Biochem J 441, 553-569 (2012)].

FIG. 9 shows heatmap of network closeness between RSK isoforms and disease modules. Heatmap of the significance of network closeness of RSK1, RSK2, RSK3 and RSK4 first neighbor modules to 44 human disorders including cardiovascular, autoimmune, metabolic and malignant diseases. The average shortest distance of the same number of randomly selected genes to disease genes was calculated for N=250 realizations in order to compare the average shortest distance value to random expectation. Empirical p-values were calculated based on 100 degree-preserved randomizations of the first neighbor networks.

FIGS. 10A and 10B show RSK1 is activated by JAK1/2 signaling in human primary macrophages in response to IFN-γ. (FIG. 10A) Human PBMC-derived macrophages were stimulated with IFN-γ for the indicated times under serum starvation. Cell lysates were subjected to immunoprecipitation with normal IgG or anti-RSK1 followed by immunoblot analysis with anti-pSer380-RSK1, anti-pSer221-RSK1, anti-pSer732-RSK1, anti-pThr573-RSK1, anti-pThr359-RSK1, or anti-RSK1. Cell lysates were subjected to immunoblot analysis with anti-pSer727-STAT1, anti-STAT1, anti-RSK1, or anti-Tubulin. (FIG. 10B) Human PBMC-derived macrophages were stimulated with IFN-γ for indicated time under serum starvation. Cells were fixed and stained with anti-RSK1. Nuclei were stained with 4,6-diamidino-2-phenylindole. N=2 donors.

FIG. 11 shows RSK1-mediated STAT1 phosphorylation at Ser727. Annotated spectrum of the pSer727-containing peptide. The spectrum was acquired using parallel reaction monitoring, PRM, using EThCD as the activation method.

FIG. 12 shows effects of silencing RSK1 on IFN-γ-induced transcription in human primary macrophages. Human PBMC-derived macrophages were treated with control siRNA or RSK1 siRNA followed by treatment with IFN-γ for indicated time. Total RNA samples were subjected to real-time PCR analysis using the indicated probes and primers. GAPDH was used for normalization. Data are means±SD from a triplicate experiment for each donor.

FIG. 13 shows effects of RSK inhibition on IFN-γ-induced production of chemokines in human macrophages. Human PBMC-derived macrophages were treated with DMSO or BI-D1870 (1 or 10 μM) for 2 hours followed by stimulation with IFN-γ for 8 hours. Total RNA samples were subjected to real-time PCR analysis using the indicated probes and primers. GAPDH was used for normalization. Data are means±SD from a triplicate experiment for each donor.

FIG. 14 shows effects of silencing RSK1 on IFN-γ-induced production of chemokines in human macrophages. Human PBMC-derived macrophages were treated with control siRNA or RSK1 siRNA followed by treatment with IFN-γ for 24 h. Culture medium was collected and then subjected to ELISA for CCL2/MCP-1, CCL7/MCP-3, CCL8/MCP-2, CXCL9/MIG, CXCL10/IP-10, or CXCL11/I-TAC. Data are means±SD from triplicate in one donor. ND stands for ‘not detected’. N=3 donors.

FIGS. 15A-15C show RXXpS/T pattern in human IFN-γ-stimulated macrophages. (FIG. 15A) Human PBBMC-derived macrophages were treated with DMSO or 10 μM BI-D1870 for 2 hours followed by stimulation with IFN-γ for 1 hour. Cell lysates were subjected to immunoblot analysis with anti-RXXpS/T antibody. (FIG. 15B) Four ellipse Venn diagram showing numbers of detected phospho-peptides in each sample. (FIG. 15C) A flow chart of screening for RSK-substrates in human macrophages. We identified 24 candidates including RPS6 and AKT1S1/PRAS40 as RSK-substrates.

FIG. 16 shows heatmap of network closeness between RSK-substrates and disease modules. Heatmap of the significance of network closeness of RSK-substrates module to 44 human disorders including cardiovascular, autoimmune, metabolic and malignant diseases. The average shortest distance of the same number of randomly selected genes to disease genes was calculated for N=250 realizations in order to compare the average shortest distance value to random expectation. Empirical p-values were calculated based on 100 degree-preserved randomizations of the first neighbor networks.

FIG. 17 shows model for RSK1-mediated macrophage activation. In IFN-γ-stimulated macrophages, RSK1 is activated by JAK1/2 signaling through Ser380 phosphorylation and translocates to the nucleus. On the other hand, JAK1/2 phosphorylates STAT1 at Tyr701, which is essential for nuclear translocation. After nuclear translocation of STAT1, RSK1 phosphorylates STAT1 at Ser727 in the nucleus and promotes production of chemokines.

FIG. 18 shows MK-1775 is the top perturbagen (small molecule) predicted to decrease RSK1 gene transcription in the monocyte/macrophage-like cancer cell line U937. Data herein is output from the web application found on the world wide web at https://amp.pharm.mssm.edu/L1000CDS2/#/index, that provides gene expression data in response to greater than 10,000 perturbagens. RSK1 is included as the 1000 (hence L1000) genes directly monitored for responsiveness to these perturbagens. We queried the database to find perturbagens that would specifically inhibit RSK1 gene expression with minimal effect on the >12,000 genes profiles (1000 directly measured and ˜11,000 gene profiles were inferred)

FIG. 19 shows human PBMC-Mq was exposed to MK-1775 for 6 h.

FIG. 20 shows RSK1 expression effect of MK-1775 under various conditions. MK-1775 may have a U937-specific effect.

FIG. 21 shows New L1000 analysis strategy.

FIG. 22 shows steps to increase specificity of candidate compounds to RSK1. We developed a workflow to find compounds that are specific to RSK1 and have no effect on its other three gene family members RSK2, 3 and 4).

FIG. 23 shows steps to increase specificity of candidate compounds to RSK1. We developed a workflow to find compounds that are specific to RSK1 and have no effect on its other three gene family members RSK2, 3 and 4).

FIG. 24 shows “analyte-centric” computational approach. This approach focuses on a given “analyte” (i.e. a phosphorylation site on a given protein—RSK1 being an example) and determines the “perturbations” (i.e. small molecules) that result in strong changes in the phosphorylation of that analyte. For each analyte, we extract all of the 1,713 perturbations (consisting of 6 cell lines, 90 small molecules (i.e. drugs) and 3 replicates for each) to identify the drugs that cause significant changes in that phosphosite.

FIG. 25 shows “z-score consensus” results on the RSK1 (S230p) phosphorylation site. On the vertical axis are the perturbations (drugs). Each subfigure is a different cell line, and each replicate for a given drug is represented as a dot. Any dot to the left of the left grey line (marking a z-score of −2) represents a drug that significantly down-regulates RSK1 (S230p), whereas any dot to the right of the right grey line (marking a z-score of 2) represents a drug that significantly up-regulates RSK1 (S230p). If there are two or more of these dots for a given drug on either side of the |z|=2 marks, it is counted as a significant modulator of RSK1 phosphorylation at the S230 residue.

FIG. 26 shows phosphosite-drug networks derived from the P100 dataset, for each cell line. RSK1-S230p is marked with a green circle if it is part of the large connected component of that network. The links emerging from RSK1-230p can be examined to determine drugs that significantly modulate the phosphorylation of that site. For example, in the MCF7 cell line, RSK1-S230p is modulated by Staurosporine. Abbreviations-A375: Human skin malignant melanoma; A549: Non-small-cell lung carcinoma; MCF7: Breast adenocarcinoma; NPC: Neural progenitor cells; PC3: Prostate adenocarcinoma; YAPC: Pancreas carcinoma.

FIG. 27 shows phosphosite-drug networks derived from the P100 dataset, for each cell line. RSK1-S230p is marked with a green circle if it is part of the large connected component of that network. The links emerging from RSK1-230p can be examined to determine drugs that significantly modulate the phosphorylation of that site. For example, in the PC3 cell line, RSK1-S230p is modulated by the UNC-1215 compound and in the YAPC cell line, RSK1-S230p is modulated by okadaic acid, vorinostat and the compound CHIR-99021. Abbreviations-A375: Human skin malignant melanoma; A549: Non-small-cell lung carcinoma; MCF7: Breast adenocarcinoma; NPC: Neural progenitor cells; PC3: Prostate adenocarcinoma; YAPC: Pancreas carcinoma.

### DETAILED DESCRIPTION

Pro-inflammatory activation of macrophages promotes various inflammatory disorders. The underlying molecular mechanisms for macrophage activation, particularly in the context of nuclear translocation, remains obscure. Data provided herein shows a systems approach to explore novel key regulators of pro-inflammatory macrophage activation using quantitative proteomics to monitor protein translocation to the nuclei of human primary macrophages elicited with interferon γ (IFN-γ). This unbiased bioinformatics identified several candidates, including RSK1, a ribosomal protein kinase. Network analysis linked RSK1 with human gene modules for various inflammatory disorders.

Work provided herein show IFN-γ stimulation promotes RSK1 phosphorylation at Ser380 via JAK signaling, resulting in STAT1 phosphorylation at Ser727, in the nuclei of macrophages. Silencing of RSK1 hinders IFN-γ-induced secretion of pro-inflammatory chemokines in human primary macrophages. Furthermore, in a mouse model of peritonitis, compound-mediated inhibition of RSK isoforms suppressed macrophage recruitment and activations. These data provide evidence that RSK1 is a key nuclear shuttling enzyme that mediates pro-inflammatory activation of macrophages.

### Treating or Preventing an Inflammatory Disease or Disorder

One aspect of the invention is a method of treating an inflammatory disease or disorder by administering to a subject in need thereof an agent that inhibits RSK1. In on embodiment, RSK1 is inhibited in a macrophage.

Another aspect of the invention is a method of treating an inflammatory disease or disorder by administering to a subject in need thereof an agent that inhibits STAT1 phosphorylation.

RSK1 and STAT1 phosphorylation can be inhibited via directly or indirectly. Agents that target RSK1 and STAT1 phosphorylation are identified herein below.

In one embodiment, inhibition of RSK1 is the inhibition of RSK1 phosphorylation. For example, inhibition prevents the phosphorylation of Serine 380 of RSK1. Methods for determining whether an agent is effective at inhibiting phosphorylation of RSK1 are known in the art, and can be performed by using an antibody specific to the phosphorylated-form of RSK1 protein via western blotting. Further, one could assess whether the RSK1 band has shifted upwards on an SDS-PAGE gel; mobility shift (e.g., upwards or downwards) of a protein band on SDS-PAGE gel is known in the art to indicate, for example, a phosphorylated-form of the protein. Further, mass-spectrometry can be used to determine if the agent has inhibited the phosphorylation of Serine 380 of RSK1. In one embodiment, the level of RSK1 phosphorylation is reduced by at least 5, 10, 15, 20, 25, 30, 35, 40, 45, 50, 55, 60, 65, 70, 75, 80, 85, 90, 95, 99%, or more following administration of an agent that inhibits RSK1, as compared to the level of phosphorylation in an untreated control population.

In one embodiment, inhibition of RSK1 is the inhibition of RSK1's nuclear translation from the cytosol into the nucleus. Upon phosphorylation, RSK1 translocates into the nucleus where it can act upon its substrates (e.g., phosphorylate its substrates). One skilled in the art can determine if an agent has prevented nuclear translocation of RSK1 using microscopy to observe both the nucleus, e.g., using DAPI stain, and RSK1, e.g., using an anti-RSK1 antibody or live reporter of RSK1, e.g., a fluorescent fusion of RSK1. In one embodiment, the percentage of cells with nuclear RSK1 is reduced by at least 5, 10, 15, 20, 25, 30, 35, 40, 45, 50, 55, 60, 65, 70, 75, 80, 85, 90, 95, 99%, or more following administration of an agent that inhibits RSK1, as compared to the percentage of cells with nuclear RSK1 in an untreated control population.

In one embodiment, inhibition of RSK1 is the inhibition of RSK1 kinase activity. RSK1 kinase activity can be assessed by determining if RSK1's known substrates, for example, STAT1, are phosphorylated. Methods for determining whether STAT1 is phosphorylated are known in the art, and can be performed by using an antibody specific to the phosphorylated-form of STAT1 protein via western blotting. Other methods for assessing phosphorylation are described herein above. Further, kinase activity assays are known in the art and are further described in, for example, Brabek, J. and Hanks, S K, Methods Mol Biol, 2004, which is incorporated herein by reference in its entirety. In one embodiment, the level of RSK1 kinase activity is reduced by at least 5, 10, 15, 20, 25, 30, 35, 40, 45, 50, 55, 60, 65, 70, 75, 80, 85, 90, 95, 99%, or more following administration of an agent that inhibits RSK1, as compared to the level of RSK1 kinase activity in an untreated control population.

In one embodiment, inhibition of RSK1 is the inhibiting the expression level and/or activity of RSK1. RSK1 kinase activity can be assessed by determining if RSK1's known substrates, for example, STAT1, are phosphorylated. Methods for determining the level of RSK1 mRNA or protein expression include, e.g., PCR based-assays and western-blotting, respectively. Assays to determine RSK1 activity include kinase activity assays, as described herein above, and assessing if RSK1 substrates are phosphorylated as described herein above. In one embodiment, the level and/or activity of RSK1 is reduced by at least 5, 10, 15, 20, 25, 30, 35, 40, 45, 50, 55, 60, 65, 70, 75, 80, 85, 90, 95, 99%, or more following administration of an agent that inhibits RSK1, as compared to the level and/or activity of RSK1 in an untreated control population.

In one embodiment, inhibition of RSK1 is the inhibition of STAT1 phosphorylation. In one embodiment, the phosphorylation of STAT1 is at Serine 727. In one embodiment, the level of STAT1 phosphorylation is reduced by at least 5, 10, 15, 20, 25, 30, 35, 40, 45, 50, 55, 60, 65, 70, 75, 80, 85, 90, 95, 99%, or more following administration of an agent that inhibits RSK1, as compared to the level of STAT1 phosphorylation in an untreated control population.

In various embodiment, inhibition of RSK1 and/or STAT1 phosphorylation inhibits the inflammatory response. In various embodiment, inhibition of RSK1 and/or STAT1 phosphorylation suppresses IFN-γ-induced pro-inflammatory chemokines in primary macrophages. One skilled in the art can determine if an inflammatory response has occurred, or been inhibited, e.g., by assaying for pro-inflammatory cytokines and/or chemokines using standard detection techniques. Pro-inflammatory cytokines and inflammation mediators include, but are not limited to, IL-1-alpha, IL-1-beta, IL-6, IL-8, IL-11, IL-12, IL-17, IL-18, TNF-alpha, leukocyte inhibitory factor (LIF), IFN-gamma, Oncostatin M (OSM), ciliary neurotrophic factor (CNTF), TGF-beta, granulocyte-macrophage colony stimulating factor (GM-CSF), and chemokines that chemoattract inflammatory cells. In one embodiment, the inflammatory response is reduced by at least 5, 10, 15, 20, 25, 30, 35, 40, 45, 50, 55, 60, 65, 70, 75, 80, 85, 90, 95, 99%, or more following administration of an agent that inhibits RSK1, or STAT1 phosphorylation, as compared to the inflammatory response in an untreated control population. In one embodiment, the percentage of suppressed IFN-γ-induced pro-inflammatory chemokines in primary macrophages is increased by at least 5, 10, 15, 20, 25, 30, 35, 40, 45, 50, 55, 60, 65, 70, 75, 80, 85, 90, 95, 99%, or more following administration of an agent that inhibits RSK1, or STAT1 phosphorylation, as compared to the percentage of suppressed IFN-γ-induced pro-inflammatory chemokines in primary macrophages in an untreated control population.

In one embodiment, the method further comprises, prior to administration, diagnosing a subject with having an inflammatory disease or disorder. In one embodiment, the method further comprises, prior to administration, receiving results that identify a subject as having an inflammatory disease or disorder.

An inflammatory disease or disorder, e.g., a condition, is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions.

Exemplary inflammatory diseases or disorders that can be treated using methods described herein include, but are not limited to, macrophage activation syndrome, ulcerative colitis, type II diabetes, rheumatoid arthritis, juvenile idiopathic arthritis, Takayasu disease, aortic stenosis, Coffin-Lowry syndrome, pulmonary hypertension, Gaucher disease, systemic lupus erythematosus, Buerger disease, atherosclerosis, coronary artery disease, myocardial infarction, peripheral artery disease, vein graft disease, in-stent restenosis, arterioveneous fistula disease, arterial calcification, calcific aortic valve disease, Crohn's disease, vasculitis syndrome, scleroderma, rheumatic heart disease, acute lung injury, chronic obstructive pulmonary disease, acute kidney injury, stroke, neuroinflammation, and fatty liver.

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, artherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-lassociated myelopathy/tropical spactic paraparesis; Devic's disease; human immunodeficiency virus encephalopathy; human immunodeficiency virus vacuolar myelopathy; peripheral neuropathies; Guillanne-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organizing pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g. viral, bacterial, fungal, parasite or prion infections. In some embodiments, an inflammatory condition is associated with an allergic response. In some embodiments, an inflammatory condition is associated with a pollutant (e.g. asbestosis, silicosis, or berylliosis).

A subject can be identified as having or be at risk of having an inflammatory disease or disorder by a skilled clinician. Diagnostic tests useful in identifying a subject having a given inflammatory disease or disorder are known in the art, and further described herein below.

Other aspects provided herein are methods of inhibiting macrophage activation comprising administering to a subject in need thereof an effective amount of an agent that inhibits RSK1, or STAT1 phosphorylation. One skilled in the art can assess whether macrophage activation has occurred using standard techniques. For example, by assessing the presence of receptors found on an activated macrophage (e.g., TLR receptors, scavenger receptors, or Fc or complement receptors) or cytokines secreted from activated macrophages (e.g., IFNγ, TNFα, IL-1, IL-6, IL-15, IL-18, and IL-23). In one embodiment, macrophage activation is decreased by at least 5, 10, 15, 20, 25, 30, 35, 40, 45, 50, 55, 60, 65, 70, 75, 80, 85, 90, 95, 99%, or more following administration of an agent that inhibits RSK1, or STAT1 phosphorylation, as compared macrophage activation in an untreated control population.

### Agents

In various aspects, an agent that inhibits RSK1, or STAT1 phosphorylation is administered to a subject having, or at risk of having an inflammatory disease or disorder. In various other aspects, an agent that inhibits RSK1, or STAT1 phosphorylation is administered to a subject to inhibit macrophage activation. In one embodiment, the agent that inhibits RSK1 or STAT1 is a small molecule, an antibody or antibody fragment, a peptide, an antisense oligonucleotide, a genome editing system, or an RNAi.

An agent can inhibit e.g., the transcription, or the translation of RSK1 in the cell. An agent can inhibit the activity or alter the activity (e.g., such that the activity no longer occurs, or occurs at a reduced rate) of RSK1 in the cell (e.g., RSK1's expression). An agent can inhibit post-translational modifications, for example, phosphorylation, of a protein (e.g., RSK1 or STAT1), interfering with the wild-type function of the protein.

The agent may function directly in the form in which it is administered. Alternatively, the agent can be modified or utilized intracellularly to produce something which inhibits RSK1, or STAT1 phosphorylation, such as introduction of a nucleic acid sequence into the cell and its transcription resulting in the production of the nucleic acid and/or protein inhibitor of RSK1, or STAT1 phosphorylation. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits RSK1. Methods for screening small molecules are known in the art and can be used to identify a small molecule that is efficient at, for example, decreasing macrophage activation, given the desired target (e.g., RSK1).

In one embodiment, the agent that inhibit RSK1 is selected from Table 1.

TABLE 1
Agents that alter RSK1 expression.

| pert_iname | pert_type | RSK1 |
|---|---|---|
| RPS6KA1 | trt_sh | −4.89 |
| RPS6KA1 | trt_sh | −4.54 |
| BUB1B | trt_sh | −3.13 |
| LCK | trt_sh | −2.75 |
| GNPDA1 | trt_sh | −2.06 |
| ERBB3 | trt_sh | −1.98 |
| ATP6V0B | trt_sh | −1.81 |
| F-1566-0341 | trt_cp | −1.80 |
| POLR2A | trt_sh | −1.77 |
| BRD-K92317137 | trt_cp | −1.71 |
| SUZ12 | trt_sh | −1.66 |
| GPR56 | trt_sh | −1.65 |
| NMT1 | trt_sh | −1.54 |
| LOXL1 | trt_sh | −1.51 |
| ARHGEF5 | trt_sh | −1.50 |
| manumycin-a | trt_cp | −1.48 |
| cerulenin | trt_cp | −1.40 |
| LDN-193189 | trt_cp | −1.38 |
| SUZ12 | trt_sh | −1.35 |
| RPS6KA1 | trt_sh | −1.34 |
| lacZ | ctl_vector | −1.28 |
| NVP-AUY922 | trt_cp | −1.23 |
| avicin-g | trt_cp | −1.17 |
| tanespimycin | trt_cp | −1.17 |
| OSI-027 | trt_cp | −1.06 |
| WDR61 | trt_sh | −1.05 |
| RFP | ctl_vector | −1.02 |
| BRD-K68548958 | trt_cp | −0.99 |
| salermide | trt_cp | −0.98 |
| BRD-K73261812 | trt_cp | −0.97 |
| tosedostat | trt_cp | −0.96 |
| chaetocin | trt_cp | −0.93 |
| MW-A1-12 | trt_cp | −0.93 |
| ZSTK-474 | trt_cp | −0.73 |
| cyclosporin-a | trt_cp | −0.69 |
| BRD-K08663380 | trt_cp | −0.65 |
| tanespimycin | trt_cp | −0.59 |
| PI-103 | trt_cp | −0.54 |
| RFP | ctl_vector | −0.52 |
| AAGTTGG | trt_sh.css | −0.52 |
| sulforaphane | trt_cp | −0.50 |

In Table 1, “cp” indicates a small molecule, “sh” indicates an shRNA, and “ctl vector” indicates a control vector. Control vectors are not designed, for example, to target RSK1 and can be used to assess an off-target effect of a vector.

In one embodiment, the small molecule that inhibits RSK1 is selected from Table 2.

TABLE 2
Small molecules that inhibit RSK1 expression.

| pert_iname | MOA | RPS6KA1 |
|---|---|---|
| manumycin-a | Unknown | −1.48 |
| cerulenin | fatty acid synthase inhibitor | −1.40 |
| tanespimycin | HSP inhibitor | −1.17 |
| salermide | Unknown | −0.98 |
| tosedostat | peptidase inhibitor | −0.96 |

In Table 2, MOA, or “mechanism of action,” indicates the class or type of small molecule tested. It is specifically contemplated herein that another small molecule having the same or similar MOAs known in the art can be used to treat an inflammatory disease or disorder, given that it targets RSK1. Accordingly, in one embodiment, the small molecule that inhibits RSK1 is a fatty acid synthase inhibitor. In another embodiment, the small molecule that inhibits RSK1 is a HSP inhibitor. And in another embodiment, the small molecule that inhibits RSK1 is a peptidase inhibitor. The mechanisms of action listed in Table 2 are in no way meant to be limiting; other mechanisms of action for the small molecules listed in Table 2 are known in the art, and are specifically contemplated herein.

In another embodiment, the small molecule is MK-1775. MK-1775 belongs to a class of tyrosine inhibitors; MK-1775 specifically inhibits the tyrosine WEE1. Accordingly, in one embodiment, the small molecule that inhibits RSK1 is a tyrosine inhibitor.

MK-1775 has a chemical compound of C₂₇H₃₂N₈O₂ and a structure of:

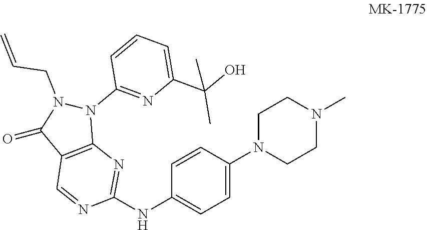 [SMILES: C=CCn1c(=O)c2cnc(Nc3ccc(N4CCN(C)CC4)cc3)nc2n1-c1cccc(C(C)(C)O)n1]

Manumycin-a is also known in the art as N-[(1S,5S,6R)-5-hydroxy-5-[(1E,3E,5E)-7-[(2-hydroxy-5-oxo-1-cyclopenten-1-yl)amino]-7-oxo-1,3,5-heptatrien-1-yl]-2-oxo-7-oxabicyclo [4.1.0]hept-3-en-3-yl]-2E,4E,6R-trimethyl,2,4-decadienamide, and has a structure of:

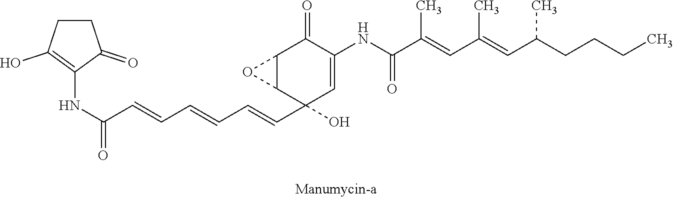

Cerulenin is also known in the art as (2R,3S)-3-[(4E,7E)-1-Oxo-4,7-nonadien-1-yl]-2-oxiranecarboxamide, and has a structure of:

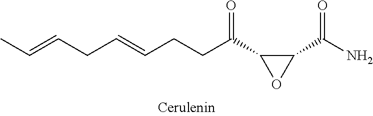 [SMILES: C/C=C/C/C=C/CCC(=O)[C@H]1O[C@H]1C(N)=O]

Tanespimycin is also known in the art as 17-N-allylamino-17-demethoxygeldanamycin, or 17-AAG, and has a structure of:

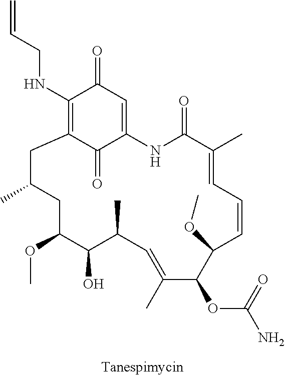 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

Salermide is also known in the art as N-[3-[[2-hydroxy-1-naphthalenyl)methylene]amino]phenyl]-a-methyl-benzeneacetamide, and has a structure of:

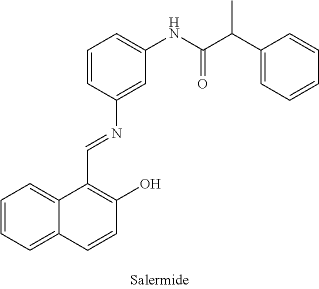 [SMILES: CC(C(=O)Nc1cccc(/N=C/c2c(O)ccc3ccccc23)c1)c1ccccc1]

Tosedostat is also known in the art as αS-[[(2R)-2-[(1S)-1-hydroxy-2-(hydroxyamino)-2-oxoethyl]-4-methyl-1-oxopentyl]amino]-benzeneacetic acid, cyclopentyl ester, and has a structure of

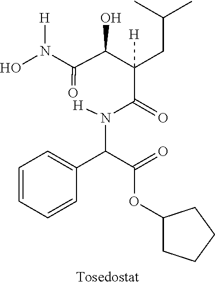 [SMILES: CC(C)C[C@@H](C(=O)NC(C(=O)OC1CCCC1)c1ccccc1)[C@H](O)C(=O)NO]

In one embodiment, the small molecule is a phosphorylation inhibitor. Specifically, the small molecule is an inhibitor of serine, or serine/threonine phosphorylation. In another embodiment, the agent is a phosphatase. A phosphatase hydrolyzes the phosphoester bonds of phosphoserines, phosphothreonines or phosphotyrosines, removing the phosphorylation of the protein. Exemplary phosphatases include, but are not limited to, Protein Phosphatase 1 (PP1), Protein Phosphatase 2A (PP2A), Protein Phosphatase 2B (PP2B), Protein Phosphatase 2C (PP2C), Protein Phosphatase 4 (PP4), Protein Phosphatase 5 (PP5), Protein Phosphatase 6 (PP6), and Protein Phosphatase 7 (PP7). In one embodiment, the phosphatase is a nucleic acid that encodes a phosphatase, or a polypeptide encoding a phosphatase. The phosphatase can be comprised within a vector for expression in a cell.

A phosphorylation inhibitor or phosphatase can be used in methods described herein to inhibit the phosphorylation of RSK1, and/or STAT1 phosphorylation.

Further, in one embodiment, the small molecule is a derivative of any of the small molecules described herein. In one embodiment, the small molecule is a variant or analog of any of the small molecules described herein. For example, the small molecule that inhibits RSK1 is a derivative of MK-1775, Manumycin-a, Cerulenin, Tanespimycin, salermide, and tosedostat. A molecule is said to be a “derivative” of another molecule when it contains additional chemical moieties not normally a part of the molecule and/or when it has been chemically modified. Such moieties can improve the molecule's expression levels, enzymatic activity, solubility, absorption, biological half-life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990). A “variant” of a molecule is meant to refer to a molecule substantially similar in structure and function to either the entire molecule, or to a fragment thereof. A molecule is said to be “substantially similar” to another molecule if both molecules have substantially similar structures and/or if both molecules possess a similar biological activity. Thus, provided that two molecules possess a similar activity, they are considered variants as that term is used herein even if the structure of one of the molecules not found in the other, or if the structure is not identical. An “analog” of a molecule is meant to refer to a molecule substantially similar in function to either the entire molecule or to a fragment thereof.

In various embodiments, the agent that inhibits RSK1, or STAT1 phosphorylation is an antibody or antigen-binding fragment thereof, or an antibody reagent that is specific for RSK1, or STAT1 phosphorylation site, e.g., STAT1 Serine 727. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the binding of the antibody inhibits the phosphorylation of RSK1 at Serine 380. In one embodiment, the binding of the antibody inhibits the phosphorylation of STAT1 at Serine 727.

In one embodiment, the agent that inhibits RSK1, or STAT1 phosphorylation is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use. Methods for humanizing a non-human antibody are known in the art.

Exemplary antibodies, for example, that are useful in inhibiting RSK1, and/or STAT1 phosphorylation (e.g., anti-RSK1 antibodies), are further described herein below in the Examples. These antibodies can further be humanized and used in the claimed methods and compositions herein.

In one embodiment, the antibody or antibody reagent binds to an amino acid sequence that corresponds to the amino acid sequence encoding RSK1 (SEQ ID NO: 2).

(SEQ ID NO: 2)
MEQDPKPPRLRLWALIPWLPRKQRPRISQTSLPVPGPGSGPQRD
SDEGVLKEISITHHVKAGSEKADPSHFELLKVLGQGSFGKVFLVRKVTR
PDSGHLYAMKVLKKATLKVRDRVRTKMERDILADVNHPFVVKLHYAFQT
EGKLYLILDFLRGGDLFTRLSKEVMFTEEDVKFYLAELALGLDHLHSLG
IIYRDLKPENILLDEEGHIKLTDFGLSKEAIDHEKKAYSFCGTVEYMAP
EVVNRQGHSHSADWWSYGVLMFEMLIGSLPFQGKDRKETMTLILKAKLG
MPQFLSTEAQSLLRALFKRNPANRLGSGPDGAEEIKRHVFYSTIDWNKL
YRREIKPPFKPAVAQPDDTFYFDTEFTSRTPKDSPGIPPSAGAHQLFRG
FSFVATGLMEDDGKPRAPQAPLHSVVQQLHGKNLVFSDGYVVKETIGVG
SYSECKRCVHKATNMEYAVKVIDKSKRDPSEEISILLRYGQHPNIITLK
DVYDDGKHVYLVTELMRGGELLDKILRQKFFSEREASFVLHTIGKTVEY
LHSQGVVHRDLKPSNILYVDESGNPECLRICDFGFAKQLRAENGLLMTP
CYTANFVAPEVLKRQGYDEGCDIWSLGILLYTMLAGYTPFANGPSDTPE
EILTRIGSGKFTLSGGNWNTVSETAKDLVSKMLHVDPHQRLTAKQVLQH
PWVTQKDKLPQSQLSHQDLQLVKGAMAATYSALNSSKPTPQLKPIESSI
LAQRRVRKLPSTTL

In another embodiment, the anti-RSK1 antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-RSK1 antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., RSK1, and for example, decreases macrophage activation.

In one embodiment, the antibody or antibody reagent binds to an amino acid sequence that corresponds to the amino acid sequence encoding STAT1 (SEQ ID NO: 4).

(SEQ ID NO: 4)
MSQTNYELQQLDSKFLEQVHQLYDDSFPMEIRQYLAQWLEKQDWE
HAANDVSFATIRFHDLLSQLDDQYSRFSLENNFLLQHNIRKSKRNLQDN
FQEDPIQMSMIIYSCLKEERKILENAQRFNQAQSGNIQSTVMLDKQKEL
DSKVRNVKDKVMCIEHEIKSLEDLQDEYDFKCKTLQNREHETNGVAKSD
QKQEQLLLKKMYLMLDNKRKEVVHKIIELLNVTELTQNALINDELVEWK
RRQQSACIGGPPNACLDQLQNWFTIVAESLQQVRQQLKKLEELEQKYTY
EHDPITKNKQVLWDRTFSLFQQLIQSSFVVERQPCMPTHPQRPLVLKTG
VQFTVKLRLLVKLQELNYNLKVKVLFDKDVNERNTVKGFRKFNILGTHT
KVMNMEESTNGSLAAEFRHLQLKEQKNAGTRTNEGPLIVTEELHSLSFE
TQLCQPGLVIDLETTSLPVVVISNVSQLPSGWASILWYNMLVAEPRNLS
FFLTPPCARWAQLSEVLSWQFSSVTKRGLNVDQLNMLGEKLLGPNASPD
GLIPWTRFCKENINDKNFPFWLWIESILELIKKHLLPLWNDGCIMGFIS
KERERALLKDQQPGTFLLRFSESSREGAITFTWVERSQNGGEPDFHAVE
PYTKKELSAVTFPDIIRNYKVMAAENIPENPLKYLYPNIDKDHAFGKYY
SRPKEAPEPMELDGPKGTGYIKTELISVSEVHPSRLQTTDNLLPMSPEE
FDEVSRIVGSVEFDSMMNTV

In another embodiment, the anti-STAT1 antibody or antibody reagent binds to an amino acid sequence that comprises the sequence of SEQ ID NO: 4; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 4. In one embodiment, the anti-STAT1 antibody or antibody reagent binds to an amino acid sequence that comprises the entire sequence of SEQ ID NO: 4. In another embodiment, the antibody or antibody reagent binds to an amino acid sequence that comprises a fragment of the sequence of SEQ ID NO: 4, wherein the fragment is sufficient to bind its target, e.g., STAT1, and for example, decreases macrophage activation

In one embodiment, the agent that inhibits RSK1, or STAT1 phosphorylation is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., RSK1, or STAT1 phosphorylation. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits RSK1, or STAT1 phosphorylation may comprise at least 5, at least 10, at least 15, at least 20, at least 25, at least 30, or more bases complementary to a portion of the coding sequence of the human RSK1 gene (e.g., SEQ ID NO: 1) or STAT1 gene (e.g., SEQ ID NO: 3).

| SEQ ID NO: 1 is a nucleotide sequence that encodes RSK1. | |
|---|---|
| (SEQ ID NO: 1) | |
| | a tggagcagga |
| 61 | tcccaagccg ccccgtctgc ggctctgggc cctgatcccc tggcttccca ggaagcagcg |
| 121 | gcccaggatc agccagacct ctctgcctgt ccctggccct ggctctggcc cccagcggga |
| 181 | ctcggatgag ggcgtcctca aggagatctc catcacgcac cacgtcaagg ctggctctga |
| 241 | gaaggctgat ccatcccatt tcgagctcct caaggttctg ggccagggat cctttggcaa |
| 301 | agtcttcctg gtgcggaaag tcacccggcc tgacagtggg cacctgtatg ctatgaaggt |
| 361 | gctgaagaag gcaacgctga aagtacgtga ccgcgtccgg accaagatgg agagagacat |
| 421 | cctggctgat gtaaatcacc cattcgtggt gaagctgcac tatgccttcc agaccgaggg |
| 481 | caagctctat ctcattctgg acttcctgcg tggtggggac ctcttcaccc ggctctcaaa |
| 541 | agaggtgatg ttcacggagg aggatgtgaa gttttacctg gccgagctgg ctctgggcct |
| 601 | ggatcacctg cacagcctgg gtatcattta cagagacctc aagcctgaga acatccttct |
| 661 | ggatgaggag ggccacatca aactcactga ctttggcctg agcaaagagg ccattgacca |
| 721 | cgagaagaag gcctattctt tctgcgggac agtggagtac atggcccctg aggtcgtcaa |
| 781 | ccgccagggc cactcccata gtgcggactg gtggtcctat ggggtgttga tgtttgagat |
| 841 | gctgacgggc tccctgccct tccaggggaa ggaccggaag gagaccatga cactgattct |
| 901 | gaaggcgaag ctaggcatgc cccagtttct gagcactgaa gcccagagcc tcttgcgggc |
| 961 | cctgttcaag cggaatcctg ccaaccggct cggctccggc cctgatgggg cagaggaaat |
| 1021 | caagcggcat gtcttctact ccaccattga ctggaataag ctataccgtc gtgagatcaa |
| 1081 | gccacccttc aagccagcag tggctcagcc tgatgacacc ttctactttg acaccgagtt |
| 1141 | cacgtcccgc acacccaagg attccccagg catccccccc agcgctgggg cccatcagct |
| 1201 | gttccggggc ttcagcttcg tggccaccgg cctgatggaa gacgacggca agcctcgtgc |
| 1261 | cccgcaggca cccctgcact cggtggtaca gcaactccat gggaagaacc tggtttttag |
| 1321 | tgacggctac gtggtaaagg agacaattgg tgtgggctcc tactctgagt gcaagcgctg |
| 1381 | tgtccacaag gccaccaaca tggagtatgc tgtcaaggtc attgataaga gcaagcggga |
| 1441 | tccttcagaa gagattgaga ttcttctgcg gtatggccag caccccaaca tcatcactct |
| 1501 | gaaagatgtg tatgatgatg gcaaacacgt gtacctggtg acagagctga tgcggggtgg |
| 1561 | ggagctgctg gacaagatcc tgcggcagaa gttcttctca gagcgggagg ccagctttgt |
| 1621 | cctgcacacc attggcaaaa ctgtggagta tctgcactca cagggggttg tgcacaggga |
| 1681 | cctgaagccc agcaacatcc tgtatgtgga cgagtccggg aatcccgagt gcctgcgcat |
| 1741 | ctgtgacttt ggttttgcca aacagctgcg ggctgagaat gggctcctca tgacaccttg |
| 1801 | ctacacagcc aactttgtgg cgcctgaggt gctgaagcgc cagggctacg atgaaggctg |
| 1861 | cgacatctgg agcctgggca ttctgctgta caccatgctg gcaggatata ctccatttgc |
| 1921 | caacggtccc agtgacacac cagaggaaat cctaacccgg atcggcagtg ggaagtttac |
| 1981 | cctcagtggg ggaaattgga acacagtttc agagacagcc aaggacctgg tgtccaagat |
| 2041 | gctacacgtg gatccccacc agcgcctcac agctaagcag gttctgcagc atccatgggt |
| 2101 | cacccagaaa gacaagcttc cccaaagcca gctgtcccac caggacctac agcttgtgaa |
| 2161 | gggagccatg gctgccacgt actccgcact caacagctcc aagcccaccc cccagctgaa |
| 2221 | gcccatcgag tcatccatcc tggcccagcg gcgagtgagg aagttgccat ccaccaccct |
| 2281 | gtga |
| SEQ ID NO: 3 is a nucleotide sequence that encodes STAT1. | |
| (SEQ ID NO: 3) | |
| | at gtctcagtgg tacgaacttc agcagcttga |
| 421 | ctcaaaattc ctggagcagg ttcaccagct ttatgatgac agttttccca tggaaatcag |
| 481 | acagtacctg gcacagtggt tagaaaagca agactgggag cacgctgcca atgatgtttc |
| 541 | atttgccacc atccgttttc atgacctcct gtcacagctg gatgatcaat atagtcgctt |
| 601 | ttctttggag aataacttct tgctacagca taacataagg aaaagcaagc gtaatcttca |
| 661 | ggataatttt caggaagacc caatccagat gtctatgatc atttacagct gtctgaagga |
| 721 | agaaaggaaa attctggaaa acgcccagag atttaatcag gctcagtcgg ggaatattca |
| 781 | gagcacagtg atgttagaca aacagaaaga gcttgacagt aaagtcagaa atgtgaagga |
| 841 | caaggttatg tgtatagagc atgaaatcaa gagcctggaa gatttacaag atgaatatga |
| 901 | cttcaaatgc aaaaccttgc agaacagaga acacgagacc aatggtgtgg caaagagtga |
| 961 | tcagaaacaa gaacagctgt tactcaagaa gatgtattta atgcttgaca ataagagaaa |
| 1021 | ggaagtagtt cacaaaataa tagagttgct gaatgtcact gaacttaccc agaatgccct |
| 1081 | gattaatgat gaactagtgg agtggaagcg gagacagcag agcgcctgta ttggggggcc |
| 1141 | gcccaatgct tgcttggatc agctgcagaa ctggttcact atagttgcgg agagtctgca |
| 1201 | gcaagttcgg cagcagctta aaaagttgga ggaattggaa cagaaataca cctacgaaca |
| 1261 | tgaccctatc acaaaaaaca aacaagtgtt atgggaccgc accttcagtc ttttccagca |
| 1321 | gctcattcag agctcgtttg tggtggaaag acagccctgc atgccaacgc accctcagag |
| 1381 | gccgctggtc ttgaagacag gggtccagtt cactgtgaag ttgagactgt tggtgaaatt |
| 1441 | gcaagagctg aattataatt tgaaagtcaa agtcttattt gataaagatg tgaatgagag |
| 1501 | aaatacagta aaaggattta ggaagttcaa cattttgggc acgcacacaa aagtgatgaa |
| 1561 | catggaggag tccaccaatg gcagtctggc ggctgaattt cggcacctgc aattgaaaga |
| 1621 | acagaaaaat gctggcacca gaacgaatga gggtcctctc atcgttactg aagagcttca |
| 1681 | ctcccttagt tttgaaaccc aattgtgcca gcctggtttg gtaattgacc tcgagacgac |
| 1741 | ctctctgccc gttgtggtga tctccaacgt cagccagctc ccgagcggtt gggcctccat |
| 1801 | cctttggtac aacatgctgg tggcggaacc caggaatctg tccttcttcc tgactccacc |
| 1861 | atgtgcacga tgggctcagc tttcagaagt gctgagttgg cagttttctt ctgtcaccaa |
| 1921 | aagaggtctc aatgtggacc agctgaacat gttgggagag aagcttcttg gtcctaacgc |
| 1981 | cagccccgat ggtctcattc cgtggacgag gttttgtaag gaaaatataa atgataaaaa |
| 2041 | ttttcccttc tggctttgga ttgaaagcat cctagaactc attaaaaaac acctgctccc |
| 2101 | tctctggaat gatgggtgca tcatgggctt catcagcaag gagcgagagc gtgccctgtt |
| 2161 | gaaggaccag cagccgggga ccttcctgct gcggttcagt gagagctccc gggaaggggc |
| 2221 | catcacattc acatgggtgg agcggtccca gaacggaggc gaacctgact tccatgcggt |
| 2281 | tgaaccctac acgaagaaag aactttctgc tgttactttc cctgacatca ttcgcaatta |
| 2341 | caaagtcatg gctgctgaga atattcctga gaatcccctg aagtatctgt atccaaatat |
| 2401 | tgacaaagac catgcctttg gaaagtatta ctccaggcca aaggaagcac cagagccaat |
| 2461 | ggaacttgat ggccctaaag gaactggata tatcaagact gagttgattt ctgtgtctga |
| 2521 | agttcaccct tctagacttc agaccacaga caacctgctc cccatgtctc ctgaggagtt |
| 2581 | tgacgaggtg tctcggatag tgggctctgt agaattcgac agtatgatga acacagtata |
| 2641 | g |

In one embodiment, RSK1, or STAT1 phosphorylation is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits RSK1, or STAT1 phosphorylation does so via RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. RSK1, or STAT1 phosphorylation. In some embodiments of any of the aspects, the agent is siRNA that inhibits RSK1, or STAT1 phosphorylation. In some embodiments of any of the aspects, the agent is shRNA that inhibits RSK1, or STAT1 phosphorylation.

One skilled in the art would be able to design siRNA, shRNA, or miRNA to target RSK1, or STAT1 phosphorylation, e.g., using publically available design tools. siRNA, shRNA, or miRNA is commonly made using companies such as Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions.

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits RSK1, or STAT1 phosphorylation. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., RSK1, or STAT1 phosphorylation), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., RSK1, or STAT1 phosphorylation, for its downregulation, for example by transfecting the siRNA, shRNA, or miRNA into cells and detecting the levels of a gene or gene product, and/or post-translational modification (e.g., RSK1, or STAT1 phosphorylation) found within the cell via PCR-based assay or western-blotting, respectively.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an RSK1, or STAT1 phosphorylation inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In one embodiment, the composition further comprises a pharmaceutically acceptable carrier. As used herein, the term “pharmaceutically acceptable”, and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. Each carrier must also be “acceptable” in the sense of being compatible with the other ingredients of the formulation. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The pharmaceutical formulation contains a compound of the invention in combination with one or more pharmaceutically acceptable ingredients. The carrier can be in the form of a solid, semi-solid or liquid diluent, cream or a capsule. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques. The phrase “pharmaceutically acceptable carrier or diluent” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

Compositions described herein can be formulated for any route of administration described herein below. Methods for formulating a composition for a desired administration are further discussed below.

### Administration

In some embodiments, the methods described herein relate to treating a subject having or diagnosed as having an inflammatory disease or disorder comprising administering an agent that inhibits RSK1, or STAT1 phosphorylation as described herein. Subjects having an inflammation disease or disorder can be identified by a physician using current methods (i.e. assessment of physical symptoms, blood work, etc.) of diagnosing a condition. Symptoms and/or complications of inflammation, which characterize these disease and aid in diagnosis are well known in the art and include but are not limited to, joint pain, skin rash, fatigue, and joint stiffness. Tests that may aid in a diagnosis of, e.g. inflammatory diseases or disorders, include but are not limited Erythrocyte sedimentation rate (ESR), C-reactive protein (CRP) and plasma viscosity (PV) blood tests. A family history of, e.g., inflammatory diseases or disorders, will also aid in determining if a subject is likely to have the condition or in making a diagnosis of an inflammatory diseases or disorders.

The agents described herein (e.g., an agent that inhibits RSK1, or STAT1 phosphorylation) can be administered to a subject having or diagnosed as having an inflammatory disease or disorder. In some embodiments, the methods described herein comprise administering an effective amount of an agent to a subject in order to alleviate at least one symptom of, e.g., an inflammatory disease or disorder. As used herein, “alleviating at least one symptom of an inflammatory disease or disorder” is ameliorating any condition or symptom associated with, e.g., an inflammatory disease or disorder (e.g., joint pain and/or stiffness, fatigue, and/or skin rash). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agent is administered systemically or locally (e.g., to an affected organ). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

In one embodiment, the agent, or compositions comprising an agent is administered through inhalation. Thus, in one embodiment, a composition comprising an agent described herein is formulated for aerosol delivery.

The term “effective amount” as used herein refers to the amount of an agent (e.g., an agent that inhibits RSK1, or STAT1 phosphorylation) can be administered to a subject having or diagnosed as having an inflammatory disease or disorder needed to alleviate at least one or more symptom of, e.g., an inflammatory disease or disorder. The term “therapeutically effective amount” therefore refers to an amount of an agent that is sufficient to provide, e.g., a particular anti-inflammatory effect when administered to a typical subject. An effective amount as used herein, in various contexts, would also include an amount of an agent sufficient to delay the development of a symptom of, e.g., an inflammatory disease or disorder, alter the course of a symptom of, e.g., an inflammatory disease or disorder (e.g., slowing the progression of joint stiffness and/or pain, or development of skin rash), or reverse a symptom of, e.g., (e.g., relieve joint stiffness and/or pain or clear skin rash). Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, or on-body injectors.

An agent described herein can be administered at least once a day, a week, every 3 weeks, a month, every 2 months, every 3 months, every 4 months, every 5 months, every 6 months, every 7 months, every 8 months, every 9 months, every 10 months, every 11 months, a year, or more.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring macrophage activation, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combinational Therapy

In one embodiment, the agent described herein is used as a monotherapy. In one embodiment, the agents described herein can be used in combination with other known agents and therapies for inflammatory disease or disorder. Administered “in combination,” as used herein, means that two (or more) different treatments are delivered to the subject during the course of the subject's affliction with the disorder, e.g., the two or more treatments are delivered after the subject has been diagnosed with the disorder or disease (for example, inflammatory disease or disorder) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder. Therapeutics used to treat inflammatory disease or disorder are known in the art and can be identified by a skilled physician.

When administered in combination, the agent and the at least one additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of asthma) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Aerosol Formulations

An agent that inhibits RSK1, or STAT1 phosphorylation or composition comprising an agent that inhibits RSK1, or STAT1 phosphorylation can be administered directly to the airways of a subject in the form of an aerosol or by nebulization. For use as aerosols, an agent that RSK1, or STAT1 phosphorylation in solution or suspension may be packaged in a pressurized aerosol container together with suitable propellants, for example, hydrocarbon propellants like propane, butane, or isobutane with conventional adjuvants. An agent that RSK1, or STAT1 phosphorylation can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of an agent that inhibits RSK1, or STAT1 phosphorylation. Exemplary gases including, but are not limited to, nitrogen, argon or helium can be used to high advantage.

In some embodiments, an agent that inhibits RSK1, or STAT1 phosphorylation can also be administered directly to the airways in the form of a dry powder. For use as a dry powder, a GHK tripeptide can be administered by use of an inhaler. Exemplary inhalers include metered dose inhalers and dry powdered inhalers.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

### Controlled and Delayed Release Dosage Forms

In some embodiments of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of an inflammatory disease or disorder, can be determined by the skilled practitioner. However, a treatment is considered “effective treatment,” as the term is used herein, if one or more of the signs or symptoms of, e.g., inflammatory disease or disorder, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., decreased joint pain, descreased joint stiffness, or decreased appearance of skin rash. Efficacy can also be measured by a failure of an individual to worsen as assessed by hospitalization, or need for medical interventions (i.e., progression of inflammation). Methods of measuring these indicators are known to those of skill in the art and/or are described herein.

Efficacy can be assessed in animal models of a condition described herein, for example, a mouse model or an appropriate animal model of inflammatory disease or disorder, as the case may be. When using an experimental animal model, efficacy of treatment is evidenced when a statistically significant change in a marker is observed.

Efficacy of an agent that inhibits inflammatory disease or disorder can additionally be assessed using methods described herein.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The claimed invention can further be described in the following numbered paragraphs:
1. A method of treating an inflammatory disease or disorder, the method comprising administering to a subject in need thereof an effective amount of an agent that inhibits Ribosomal S6 Kinase-1 (RSK1).
2. The method of paragraph 1, wherein inhibition of RSK1 is the inhibition of RSK1 phosphorylation.
3. The method of paragraph 2, wherein the RSK1 phosphorylation is at Serine 380.
4. The method of any of the preceding paragraphs, wherein inhibition of RSK1 is the inhibition of RSK1 nuclear translocation.
5. The method of any of the preceding paragraphs, wherein inhibition of RSK1 is the inhibition of RSK1 kinase activity.
6. The method of paragraph 5, wherein inhibition of RSK1 kinase activity inhibits the phosphorylation of Signal transducer and activator of transcription 1 (STAT1).
7. The method of paragraph 6, wherein the phosphorylation of STAT1 is at Serine 727.
8. The method of any of the preceding paragraphs, wherein inhibition of RSK1 inhibits the inflammatory response.
9. The method of any of the preceding paragraphs, further comprising, prior to administration, diagnosing a subject with having an inflammatory disease or disorder.
10. The method of any of the preceding paragraphs, further comprising, prior to administration, receiving results that identify a subject as having an inflammatory disease or disorder.
11. The method any of the preceding paragraphs, wherein the agent that inhibits RSK1 is selected from the group consisting of a small molecule, an antibody, a peptide, a genome editing system, an antisense oligonucleotide, and an RNAi.
12. The method of paragraph 11, wherein the small molecule is selected from the group consisting of: MK-1775, Manumycin-a, Cerulenin, Tanespimycin, salermide, and tosedostat.
13. The method of paragraph 11, wherein the RNAi is a microRNA, an siRNA, or a shRNA.
14. The method of paragraph 11, wherein the antibody is a humanized antibody.
15. The method of any of the preceding paragraphs, wherein inhibiting RSK1 is inhibiting the expression level and/or activity of RSK1.
16. The method of paragraph 15, wherein the expression level and/or activity of RSK1 is inhibited by at least 50%, at least 60%, at least 70%, at least 80%, at least 90%, or more as compared to an appropriate control.
17. The method of any of the preceding paragraphs, wherein inhibition of RSK1 suppresses IFN-γ-induced chemokines in primary macrophages.
18. The method of paragraph 17, wherein the IFN-γ-induced chemokines are suppressed by at least 50%, at least 60%, at least 70%, at least 80%, at least 90%, or more as compared to an appropriate control.
19. The method of any of the preceding paragraphs, further comprising administering at least a second therapeutic for an inflammatory disease or disorder.
20. A method of treating an inflammatory disease or disorder, the method comprising administering to a subject in need thereof an effective amount of an agent that inhibits Signal transducer and activator of transcription 1 (STAT1) phosphorylation.
21. The method of paragraph 20, wherein STAT1 phosphorylation is at Serine 727.
22. The method of any of the preceding paragraphs, wherein inhibition of STAT1 phosphorylation inhibits the inflammatory response.
23. The method of any of the preceding paragraphs, further comprising, prior to administration, diagnosing a subject with having an inflammatory disease or disorder.
24. The method of any of the preceding paragraphs, further comprising, prior to administration, receiving results that identify a subject as having an inflammatory disease or disorder.
25. The method of any of the preceding paragraphs, wherein the agent that inhibits STAT1 phosphorylation is selected from the group consisting of a small molecule, an antibody, a peptide, a genome editing system, an antisense oligonucleotide, and an RNAi.
26. The method of paragraph 25, wherein the RNAi is a microRNA, an siRNA, or a shRNA.
27. The method of paragraph 25, wherein the antibody is a humanized antibody.
28. The method of any of the preceding paragraphs, wherein the phosphorylation is inhibited by at least 50%, at least 60%, at least 70%, at least 80%, at least 90%, or more as compared to an appropriate control.
29. The method of any of the preceding paragraphs, further comprising administering at least a second therapeutic for an inflammatory disease or disorder.
30. The method of any of the preceding paragraphs, wherein the subject has not been previously diagnosed with or identified as having an inflammatory disease or disorder.
31. The method of any of the preceding paragraphs, wherein the subject has been previously diagnosed with or identified as having an inflammatory disease or disorder.
32. The method of any of the preceding paragraphs, wherein the inflammatory disease or disorder is selected from the group consisting of: macrophage activation syndrome, ulcerative colitis, type II diabetes, Rheumatoid arthritis, juvenile idiopathic arthritis, Takayasu disease, aortic stenosis, Coffin-Lowry syndrome, pulmonary hypertension, Gaucher disease, systemic lupus erythematosus, Buerger disease, atherosclerosis, coronary artery disease, Crohn's disease, myocardial infarction, vasculitis syndrome, and scleroderma.
33. A method of inhibiting macrophage activation, the method comprising administering to a subject in need thereof an effective amount of an agent that inhibits RSK1.
34. A method of inhibiting macrophage activation, the method comprising administering to a subject in need thereof an effective amount of an agent that inhibits STAT1 phosphorylation.
35. A composition comprising an agent that inhibits RSK1.
36. A composition comprising an agent that inhibits STAT1 phosphorylation.
37. The composition of any of the preceding paragraphs, further comprising a pharmaceutically acceptable carrier.

### EXAMPLES

### Example 1

In data presented herein, IFN-γ, representing pro-inflammatory instigators, was used to identify new molecular mechanisms contributing to macrophage activation through transcriptional activation of nuclear STAT1. While many studies have used human cell lines or mouse cells in macrophage research, we are aware that responses to stimuli may differ between cancer cells and primary cells and between species. The present study utilized human primary macrophages derived from peripheral blood mononuclear cells (PBMC) in a systems approach to identify IFN-γ-induced nuclear translocation of key regulators of macrophage activation. Our holistic target discovery platform has involved proteomics of nuclear translocation, bioinformatics for clustering, and network analysis. We discovered that RSK1 is a nuclear shuttling kinase for pro-inflammatory macrophage activation in response to IFN-γ. IFN-γ-induced RSK1 phosphorylation in turn facilitates STAT1 phosphorylation at Ser727 in the nucleus, promoting inflammatory responses. These novel findings provide insights into regulatory mechanism for inflammatory diseases.

Results

Quantitative nuclear proteomics demonstrates enrichment of nuclear-specific and nuclear shuttling proteins.

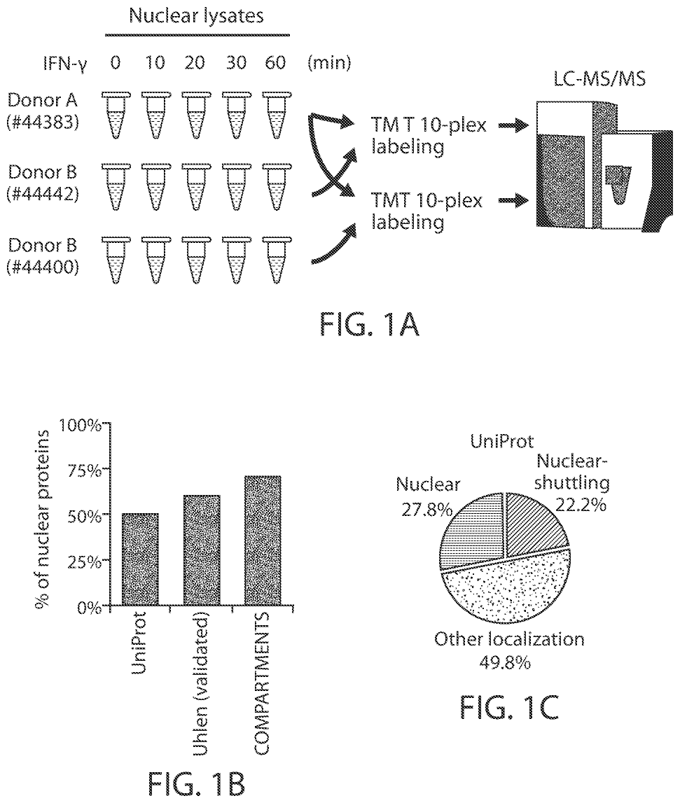

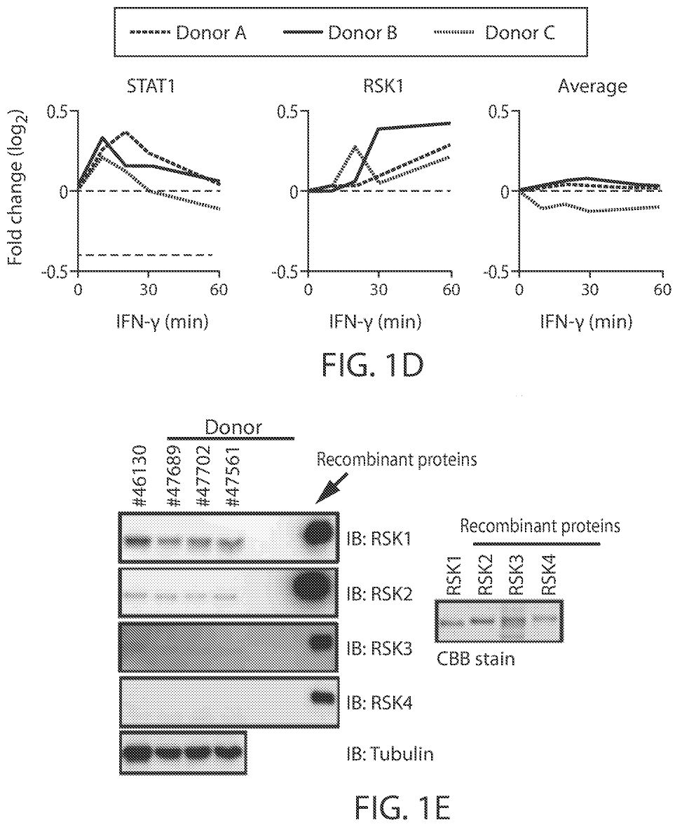

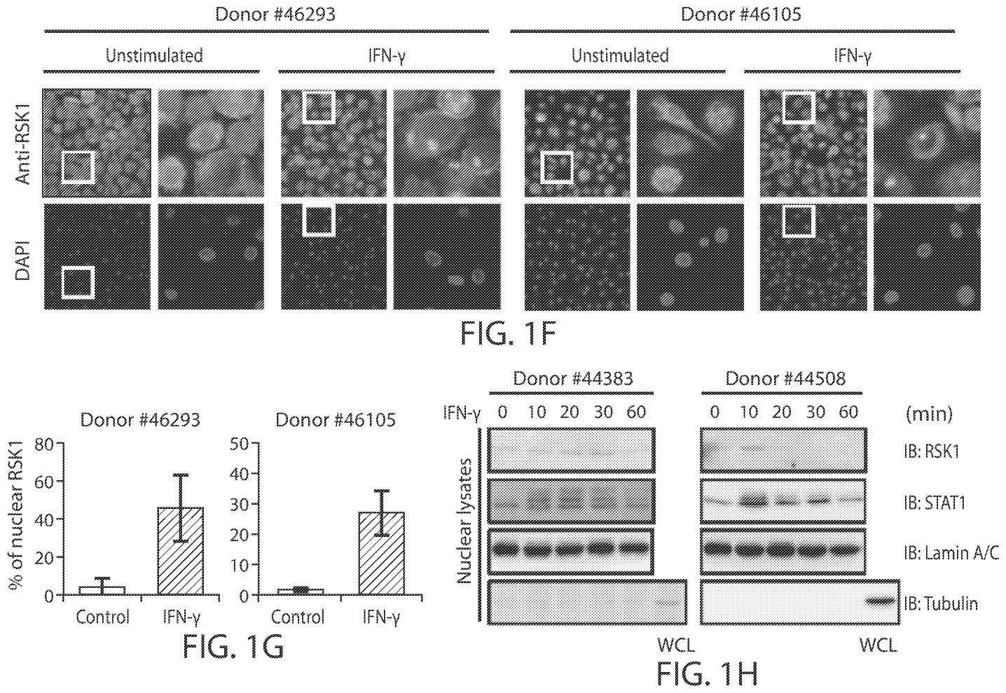

Translocation of phospho-STAT1-Tyr701 to the nucleus in response to IFN-γ is a critical step towards the STAT1-dependent expression of pro-inflammatory molecules such as chemokines. This transient increase in nuclear signal (primarily measured using immunoblotting or immunostaining) usually occurs within 60 minutes after IFN-γ treatment (14, 15). To investigate whether additional proteins are translocated to the nucleus in a similar manner, we performed quantitative nuclear translocation proteomics using human PBMC-derived primary macrophages elicited with IFN-γ for one hour. Nuclear lysates from three different PBMC donors (Donors A, B, and C) with five time points of IFN-γ stimulation (0, 10, 20, 30, and 60 minutes) were digested and labeled using isobaric tandem mass tags (TMT) followed by mass-spectrometric analysis (FIG. 1A). Two donor time-course experiments were combined in each TMT 10-plex experiment, where Donor A was run in duplicate to account for potential technical variations due to TMT batch effects (FIG. 1A). We identified a total of 1086 distinct proteins when considering the combined data from both TMT 10-plex sets. To verify that we enriched for nuclear-prominent proteins, we queried the corresponding gene identifiers against three public datasets: UniProt (16), Uhlen et al. (17), and COMPARTMENTS (18), which in term confirmed that 50.1-70.8% of detected proteins are known to be localized to the nucleus or are annotated as being localized to the nucleus and other organelles (FIGS. 1, 1B and 1C). For this latter annotation we refer to these proteins as nuclear shuttling proteins since they can be found in multiple compartments in the cell, and IFN-γ could promote their accumulation in the nucleus, a process that can be monitored by a kinetics experiment.

RSK1 is a novel IFN-γ-induced nuclear translocating protein.

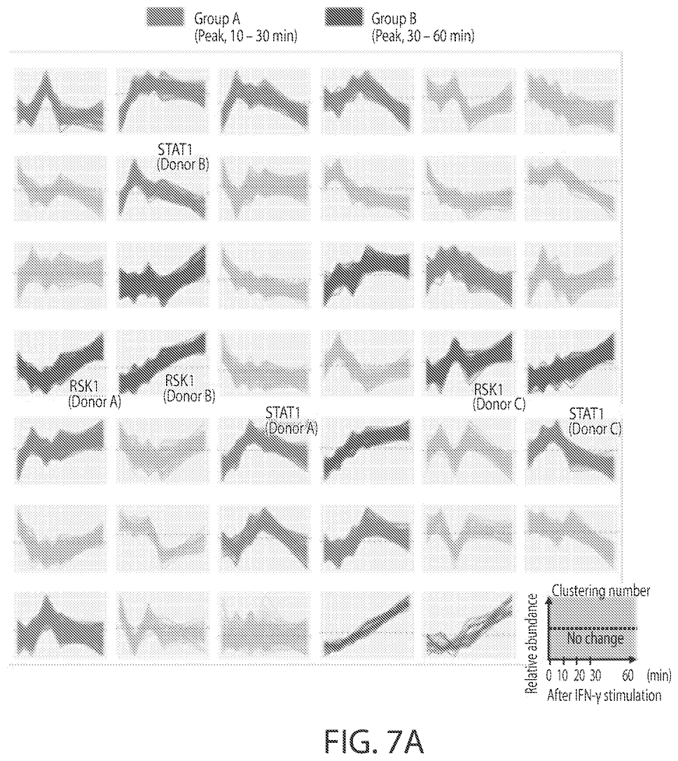

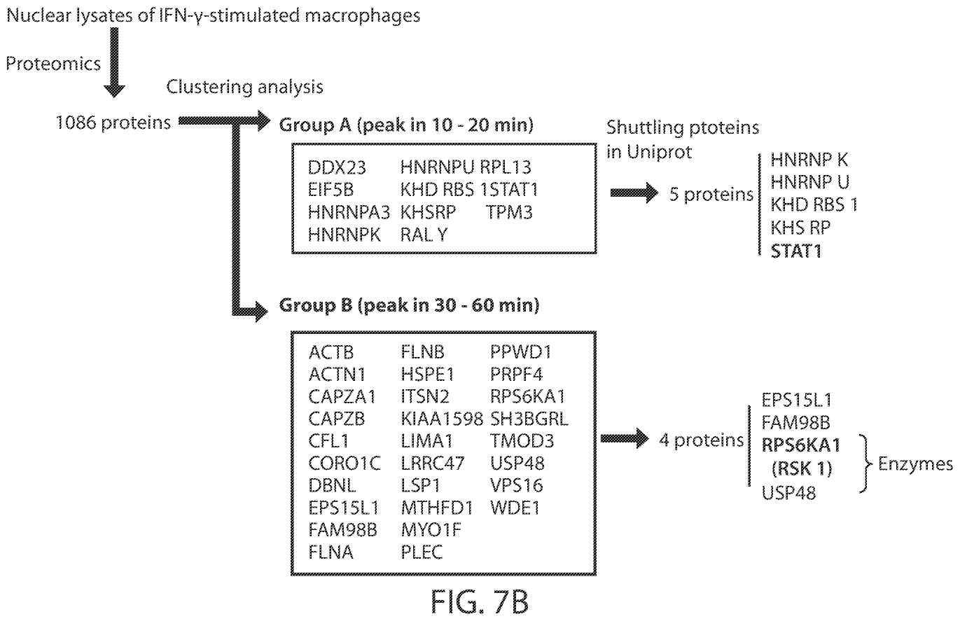

Our hypothesis is that proteins that undergo nuclear translocation in response to IFN-γ will exhibit a discernable increase in abundance at one time point (10, 20, 30, or 60 minutes) that could decline before the 60-minute mark or remain sustained. To classify the protein kinetics profiles, we performed a high-dimensional cluster analysis method (see Methods) previously published by our group (19). We combined the three donors' kinetics data into a single input for clustering (see Methods) that resulted in 41 clusters (FIG. 7A). We focused on clusters that would indicate translocation to the nucleus by an increase in abundance at a given time point followed by a decrease, or, an increase in abundance followed by a sustained signal up to 60 minutes. STAT1, for instance, appeared in three distinct clusters (clusters #8, #27 and #30), owing to the slight difference in observed kinetics across the three donors (FIG. 7A). Irrespective of the donor, however, STAT1 signal peaked between the 10 to 30-minutes time range. We therefore grouped the clusters according to their relative peak abundance timing with respect to the STAT1 control as either Group A (peaking between 10-30 minutes) or Group B (peaking between 30 to 60 minutes) (FIG. 7A).

From these two cluster groups, we further refined our list of proteins to those whose kinetic trends were similar in all three donors, and selected proteins quantified with at least five unique to include profiles with increased quantified observations (20), resulting in 11 proteins from Group A, including STAT1, and 28 proteins in Group B (FIGS. 7A and 7B). We then cross-checked these 39 proteins with the UniProt extracted annotations (FIG. 1C) for those that we characterized as nuclear shuttling proteins. This final filtering step resulted in five candidate proteins from Group A, HNRNPK, HNRNPU, KHDRBS1, KHSRP, and STAT1 that are annotated as RNA or DNA binding proteins (FIGS. 7A and 7B); and four proteins from Group B, EPS15L1, FAM98B, RPS6KA1, and USP48 that are annotated as having a variety of molecular functions including cadherin binding (EPS15L1), t-RNA processing (FAM98B), protein kinase activity (RPS6KA1), and ubiquitin hydrolase activity (USP48) (FIGS. 7A and 7B).

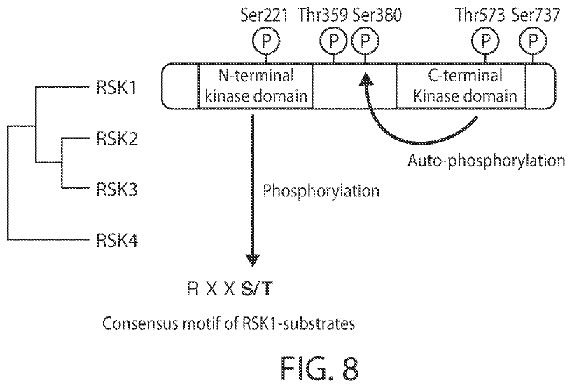

We were particularly interested RPS6KAI, ribosomal protein S6 kinase alpha-1, also known as RSK1 (FIG. 1D), because it is known to be translocated to the nuclei of HeLa cells in response to growth factor stimulation (21). This translocation leads to phosphorylation of nuclear substrates to regulate transcription of mitogen-responsive genes (21, 22). Given that our nuclear proteomics detected RSK1 translocation to the nucleus in response to IFN-γ, we therefore hypothesized that RSK1 may also phosphorylate proteins involved in transcription regulation during macrophage activation. However, RSK1 is just one of four kinases in this enzyme family, RSK1, RSK2 RSK3 and RSK4 (FIG. 8) of which RSK2 and RSK3 are also known to translocate to the nucleus in response to growth factor (23, 24). RSK4 is distinct from the other RSK isoforms in that it is predominantly cytosolic and constitutively active (25). Since we only detected RSK1 in our proteomics data (FIG. 1D), it would indicate that the three other kinases were sufficiently lower in abundance that they were not sequenced by the mass spectrometer. We therefore investigated whether they are in fact expressed in macrophages using immunoblot analysis (FIG. 1E). Compared to the equally loaded recombinant RSK standards, we could deduce the relative expression levels of the four enzymes in unstimulated macrophages, where RSK3 RSK2<RSK1, and no signal for RSK4 (FIG. 1E).

To confirm the results of the RSK1 nuclear translocation kinetics data (FIG. 1D), we performed immunofluorescence staining using human PBMC-derived macrophages to visualize translocation of RSK1 to nuclei. In unstimulated macrophages, RSK1 signal was diffused throughout the cell (FIG. 1F); however, after 30 minutes of IFN-γ stimulation, intense RSK1 signal was detected in the nuclei (FIGS. 1F and 1G). Anti-RSK1 immunoblot analysis of nuclear lysates from human macrophages also confirmed IFN-γ-induced nuclear translocation of RSK1 between 20 to 30 minutes of one donor and by 10 minutes for the second (FIG. 1H). Using multiple detection methods, proteomics and immuno-based manner, we confirmed that RSK1 is translocated to the nucleus in response to IFN-γ stimulation. Although the exact timing of this translocation can vary across donors we consistently observe that it occurs within 60 minutes of stimulation.

Network analysis links RSK1 to human inflammatory diseases.

Proteomics and immunoblot analysis indicate that, of the four RSK enzymes, RSK1 predominates in human primary macrophages (FIG. 1D). We therefore hypothesized that if RSK activity contributes to macrophage activation it is likely to occur through the most abundant RSK1. Sequence alignment indicates the four enzymes share 79.7-81.0% (>594 sequence identity) where RSK2 and RSK3 are the most similar to each other, and RSK4 is least conserved with respect to the other three enzymes (FIG. 8). Divergence in sequence conservation suggests divergence in function, not related to enzyme activity per se (since the active sites are conserved, FIG. 8), but related to signaling pathways and molecular interactions. Based on this assumption, we performed network analysis on each RSK enzyme to determine their likely molecular interactors and potential involvement in a variety of human diseases.

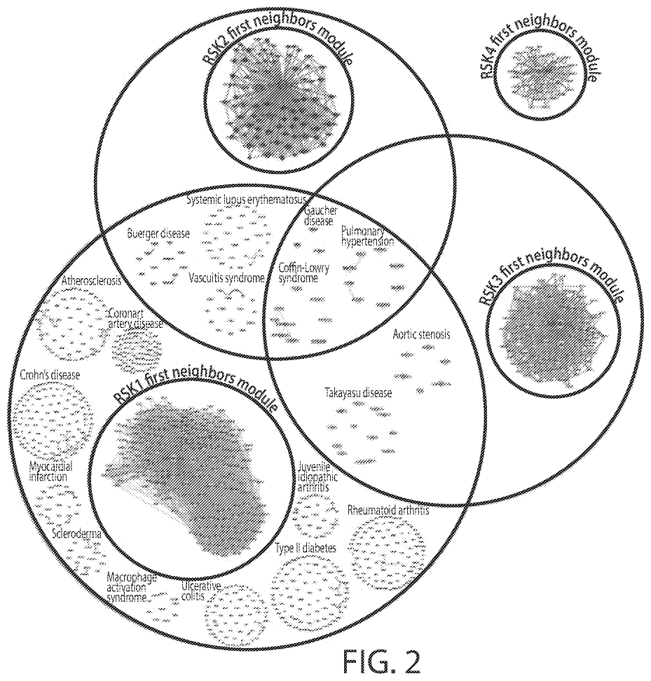

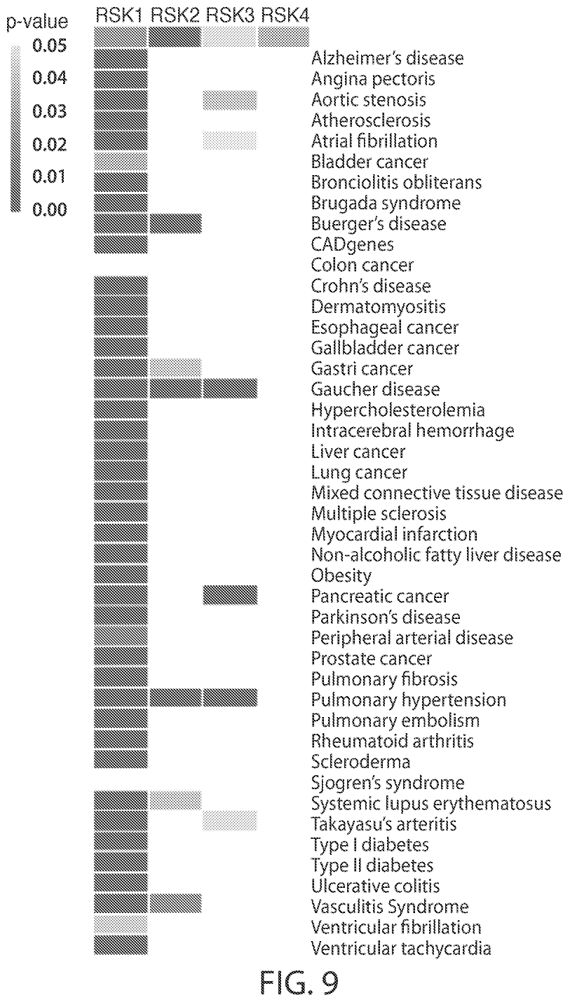

Recent evidence suggests that disease-related proteins tend to localize within the molecular interaction network, or the interactome, forming closely interacting subnetworks called disease modules (26). Furthermore, the interactome-based location of a disease determines its pathobiological relationship to other diseases (27, 28). We sought to establish the association of the RSK family of proteins with a variety of macrophage activation-associated diseases such as, cardiovascular, autoimmune, and metabolic disorders. Based on the network proximity between the RSK interaction partners and disease modules (see Methods), we identified that the RSK1-first neighbor module is significantly close to many autoimmune, cardiovascular and metabolic diseases (FIG. 2 and FIG. 9). Moreover, RSK2 and RSK3 modules share disease associations with RSK1 (FIG. 2). RSK2 and RSK3, however, tend to associate with less human disease gene modules than does RSK1. The RSK4 module shows no significant associations with any of the diseases we tested (FIG. 2 and FIG. 9). These results may predict that RSK1 has the most potential impact on human inflammatory diseases among the RSK family of proteins.

RSK1 is activated by JAK signaling in IFN-γ-stimulated macrophages.

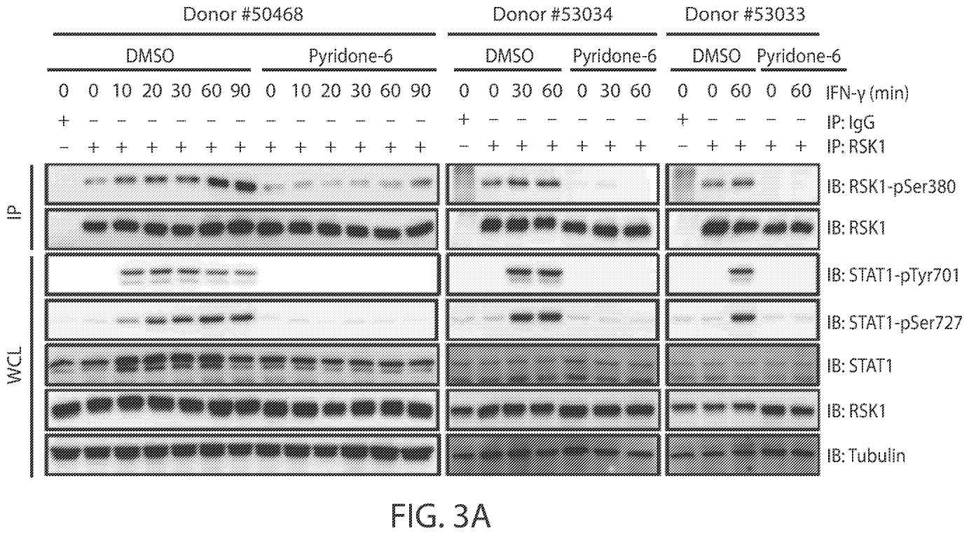

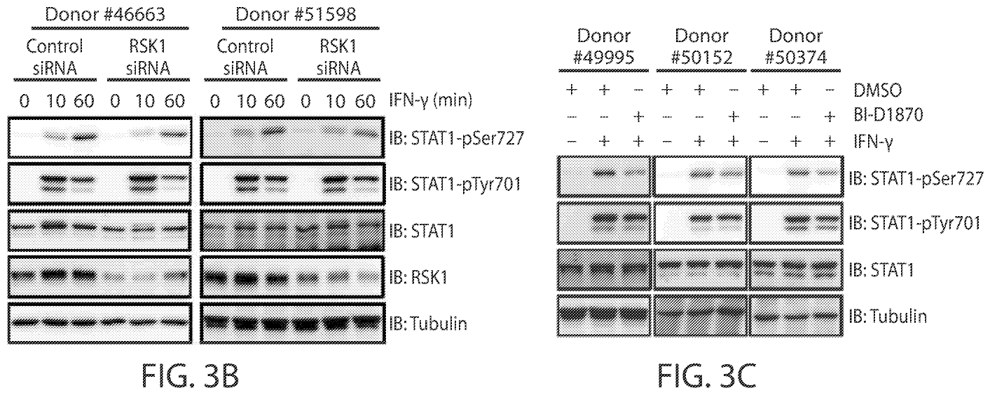

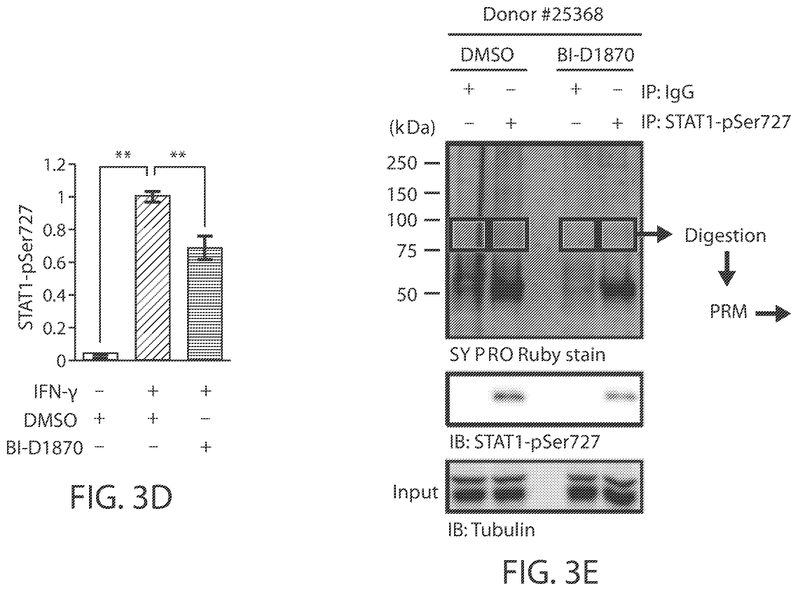

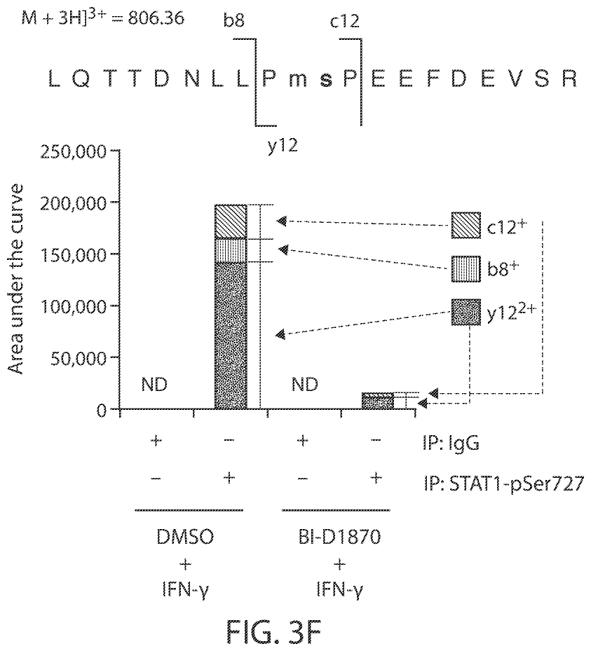

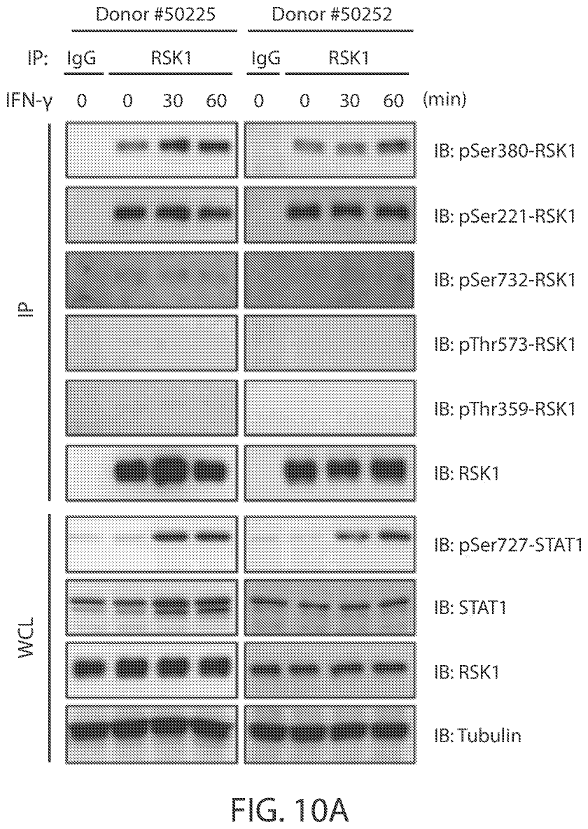

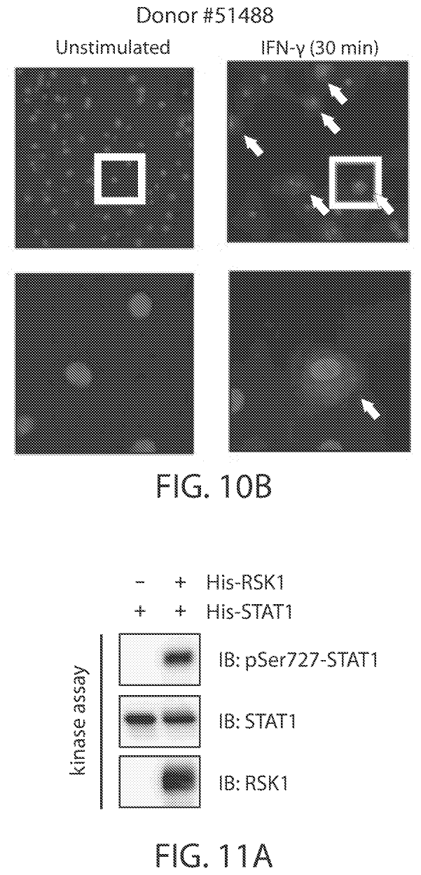

Previous studies showed that an enzymatic activity of RSK1 is regulated by the status of multiple phosphorylation sites (29). As many as five phosphorylation sites, Ser221, Thr359, Ser380, Thr573, and Ser732, have been reported for RSK1 activation in epidermal growth factor (EGF) signaling (FIG. 8). We therefore investigated whether pro-inflammatory signaling affects the phosphorylation status of RSK1 in PMBC-derived human primary macrophages. We stimulated human macrophages with IFN-γ for 30 and 60 minutes, then immunoprecipitated with anti-RSK1 antibody, followed by immunoblot analysis against these five phosphorylation sites (FIG. 10A). When compared to the unstimulated macrophages, there was increase in signal for phospho-RSK1-Ser380 between 30 and 60 minutes of IFN-γ treatment, but no change to phosphorylation of Ser221 and Ser732. Signals for phosphorylation of Thr359 and Thr573 were too low to perform any comparisons (FIG. 10A). These data imply that RSK1 is activated through Ser380 phosphorylation in pro-inflammatory activated macrophages. Moreover, immunofluorescence staining revealed that Ser380 phosphorylation mainly increased in the cytoplasm of macrophages in response to IFN-γ (FIG. 10B). To further define whether the RSK1 activation is regulated by JAK signaling, we treated human macrophages with DMSO or a pan-JAK inhibitor pyridone-6 followed by stimulation with IFN-γ for up to 90 minutes. With IFN-γ alone, phospho-RSK1-Ser380 signal increased as early as 10 minutes, but most dramatically at 60 minutes (FIG. 3A). Pyridone-6-mediated inhibition of JAK signaling resulted in a marked suppression of phospho-RSK1-Ser380 signal (FIG. 3A). This response to pyridine-6 is similar to that of phospho-STAT-Ser727, as confirmed in the corresponding cell lysates (FIG. 3A). These results indicate that JAK signaling mediates IFN-γ-induced activation of RSK1 in macrophages.

RSK1 inhibition reduces STAT1 phosphorylation at SER727 in IFN-γ-stimulated macrophages.

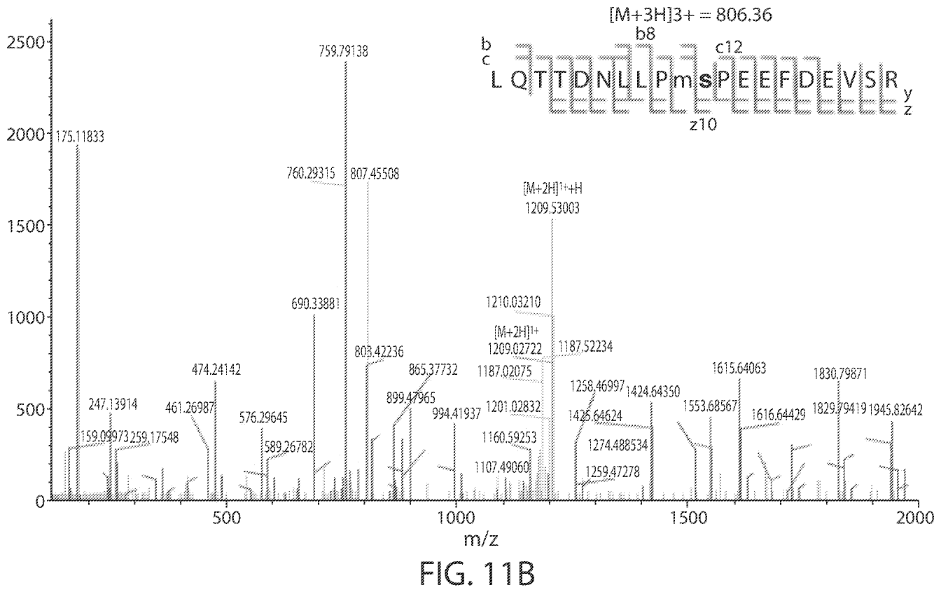

Thus far our data support that, like STAT1, RSK1 is a downstream target for JAK signaling (FIG. 3A). Given that phosphorylation of STAT1 at Ser727 occurs in the nucleus and is important for its activity (30), and that RSK1 is translocated to the nucleus, we hypothesized that STAT1 is a substrate for nuclear RSK1. We incubated recombinant RSK1 with or without STAT1 in presence of ATP for 1 hour. Immunoblot analysis confirmed that RSK1 is capable of phosphorylating STAT1 at Ser727 in vitro (FIG. 11A).

To assess whether RSK1 induces phosphorylation of endogenous STAT1 at Ser727, we transfected human PBMC-derived macrophages with control siRNA or RSK1 siRNA followed by IFN-γ exposure. In the siRNA controls, phospho-STAT1-Tyr701 signal was observed at 10 minutes followed by a decrease at 60 minutes of IFN-γ stimulation; whereas an increase of the basally phospho-STAT1-Ser727 was observed at 10 minutes, and increased further at 60 minutes (FIG. 3B), consistent with two waves of STAT1 phosphorylation dynamics reported previously (12). RSK1 silencing attenuated the phospho-STAT1-Ser727 signal, but not that of phospho-STAT1-Tyr701 (FIG. 3B), indicating that RSK1 contributes to this second wave of STAT1 phosphorylation.

In addition to siRNA, we also used an RSK inhibitor, BI-D1870 (31), to monitor the STAT1 phosphorylation status of Tyr701 and Ser727 at 60 minutes of IFN-γ stimulation. We treated human primary macrophages with DMSO (control) or BI-D1870 and confirmed the attenuated signal for phospho-STAT1-Ser727 and no change to that of phospho-STAT-Tyr701 (FIGS. 3C and 3D). To further validate RSK-mediated phospho-STAT1-Ser727 in macrophages, we performed immunoprecipitation of the cell lysates with either control IgG or anti-STAT1-pSer727 for subsequent mass spectrometric analysis (FIG. 3E). The anti-tubulin blots confirmed equal loading of cell lysate protein input to the antibody (FIG. 3E). Post-immunoprecipitation, we recovered less STAT1-pSer727 in the BI-D1870 condition versus IFN-γ (plus DMSO) alone and confirmed the pSer727 site-specific decrease in signal using parallel reaction monitoring (PRM) of three EThcD fragment ions (y12²⁺, b8⁺, and c12⁺) (FIG. 3F and FIG. 11b).

Thus, two independent methods (RSK1 siRNA and BI-D1870) support that RSK1 contributes to the levels of phospho-STAT1-Ser727 indicating that RSK1 can induce pro-inflammatory signaling events through STAT1 phosphorylation in macrophages.

RSK1 promotes secretion of inflammatory chemokines during macrophage activation.

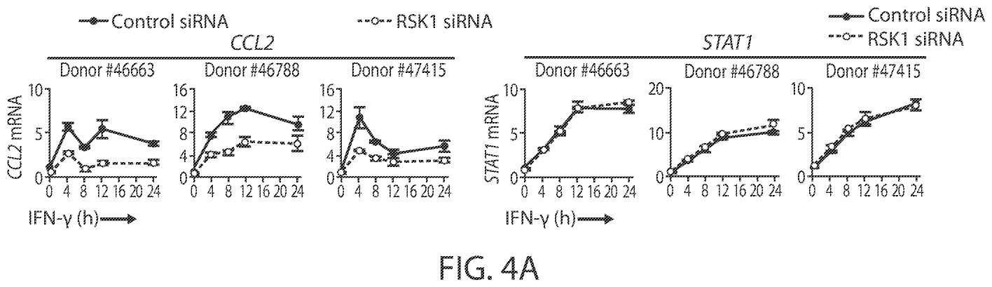

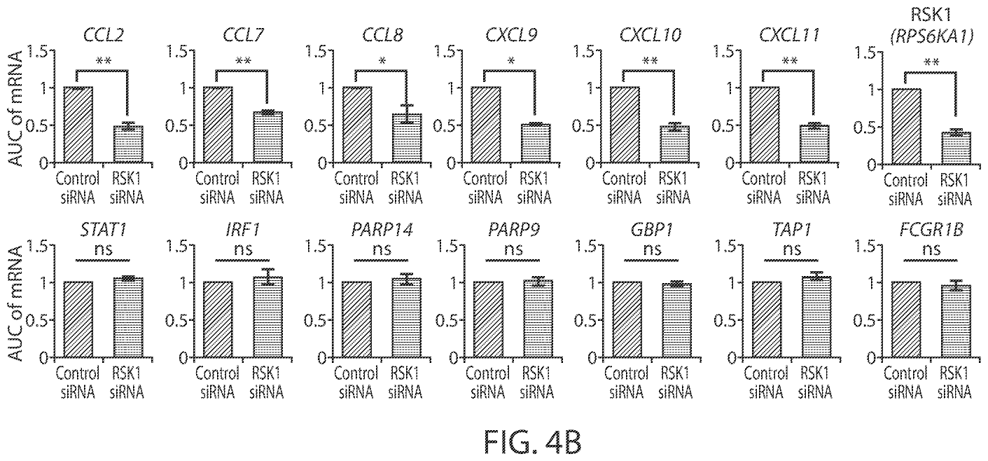

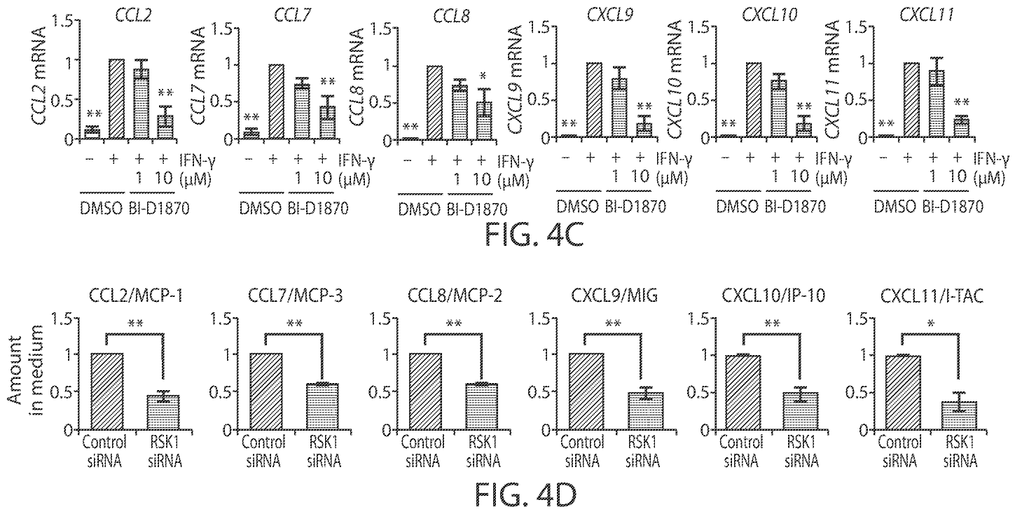

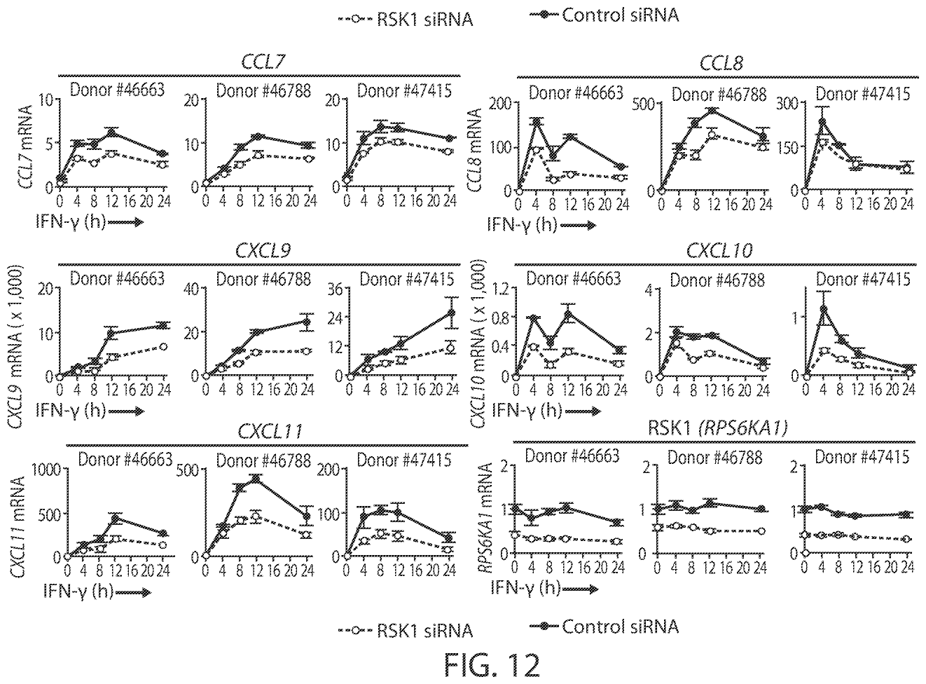

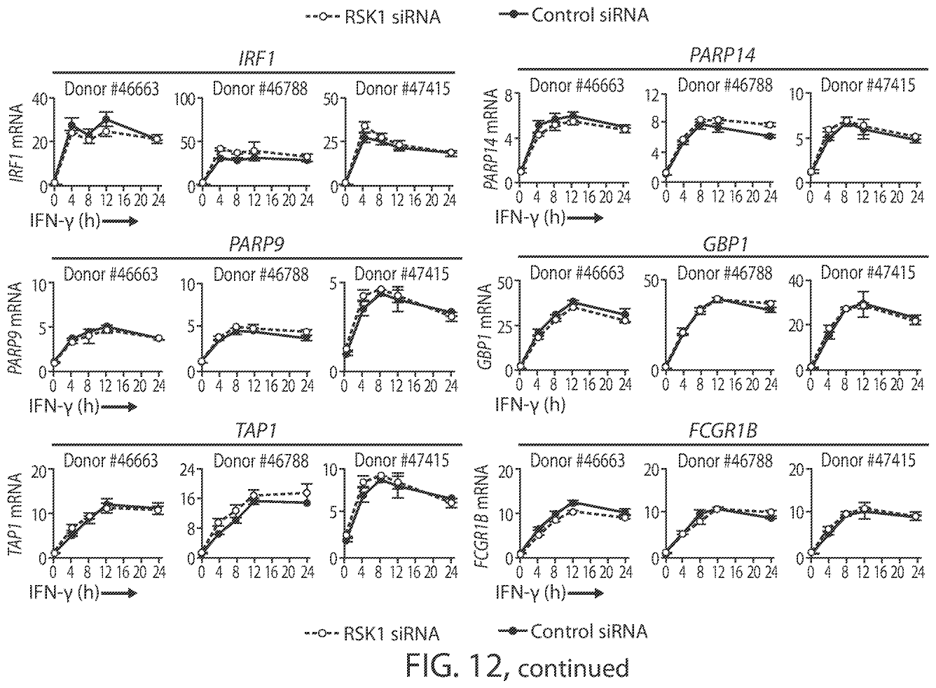

To demonstrate that RSK1 can activate macrophages through STAT1 signaling, we investigated whether RSK1 silencing decreases the transcription of IFN-γ-induced chemokine mRNA. Human primary macrophages were treated with control siRNA or RSK1 siRNA followed by IFN-γ stimulation up to 24 hours. IFN-γ increased the expression of the pro-inflammatory chemokines CCL2/MCP-1, CCL7/MCP-3, CCL8/MCP-2, CXCL9/MIG, CXCL10/IP-10, and CXCL11/I-TAC, and RSK1 silencing decreased their total expression levels throughout the IFN-γ stimulation period (FIG. 4A and FIG. 12), as demonstrated by the area-under-the-curve graphs (FIG. 4B). We also monitored the expression of other known IFN-γ-inducible genes such as those encoding transcription factors STAT1 and IRF1 (32, 33); enzymes GBP1, PARP14 and PARP9 (32-34), and membrane proteins TAP1 and FCGR1B (32). RSK1 silencing, however, exerted no effects on any of their mRNA levels (FIGS. 4A and 4B and FIG. 12). RSK1 may therefore selectively mediate the induction of a certain set of molecules in response to IFN-γ stimulation.

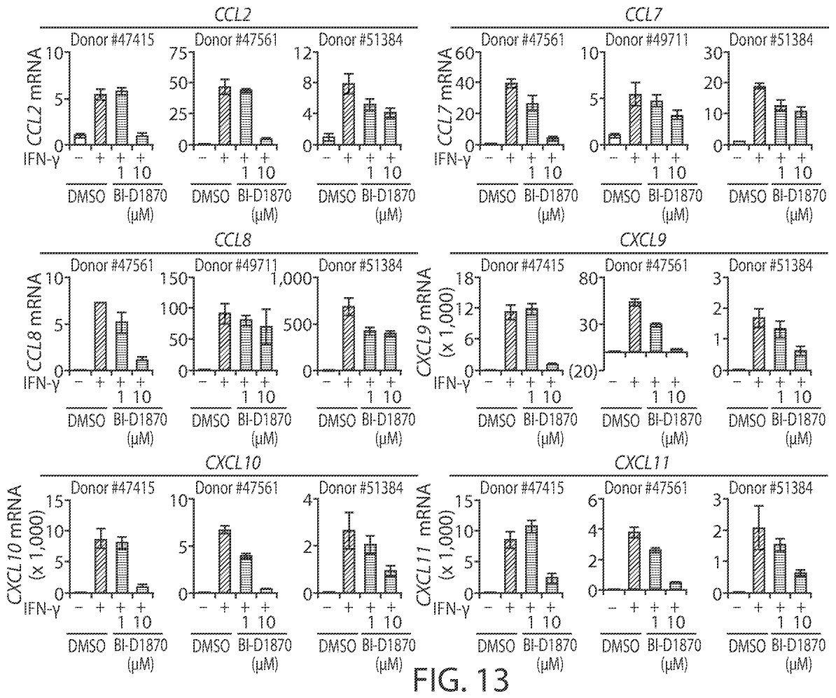

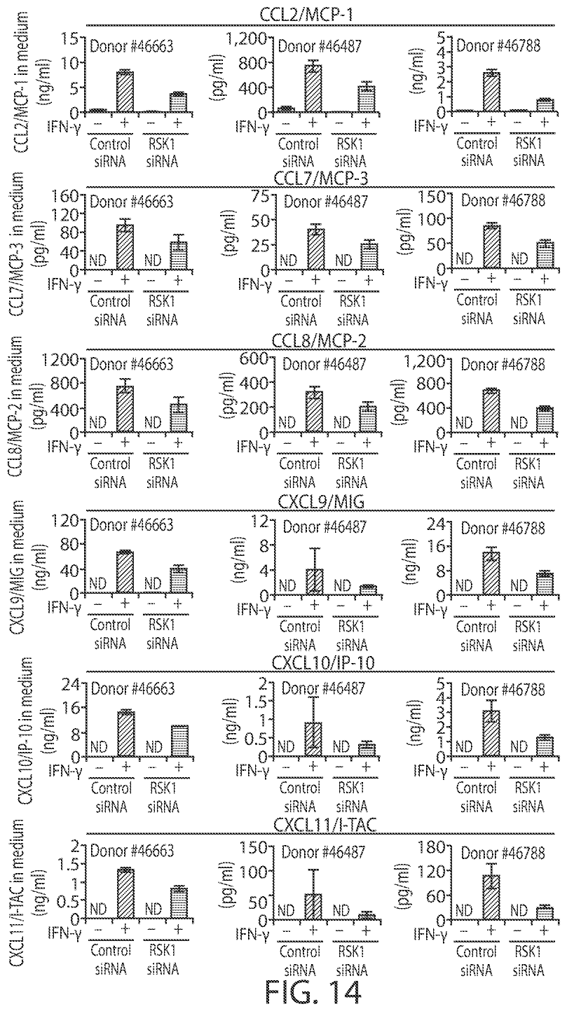

To further validate the siRNA data, we treated human primary macrophages with BI-D1870 followed by stimulation with IFN-γ. Compared to the DMSO condition, BI-D1870 attenuated mRNA levels of pro-inflammatory chemokines such as CCL2/MCP-1 (FIG. 4C and FIG. 13). This decrease in mRNA also resulted in a decrease in secreted chemokines. IFN-γ induced the release of CCL2/MCP-1, CCL7/MCP-3, CCL8/MPC-2, CXCL9/MIG, CXCL10/IP-10, and CXCL11/I-TAC into culture media of human macrophages, which was abrogated by RSK1 silencing (FIG. 4D and FIG. 14). Taken together, these data indicate that RSK1 mediates the increased secretion of major chemokines including CCL2/MCP-1 in IFN-γ-triggered macrophage activation.

RSK plays a key role in activating macrophages in peritonitis in mice.

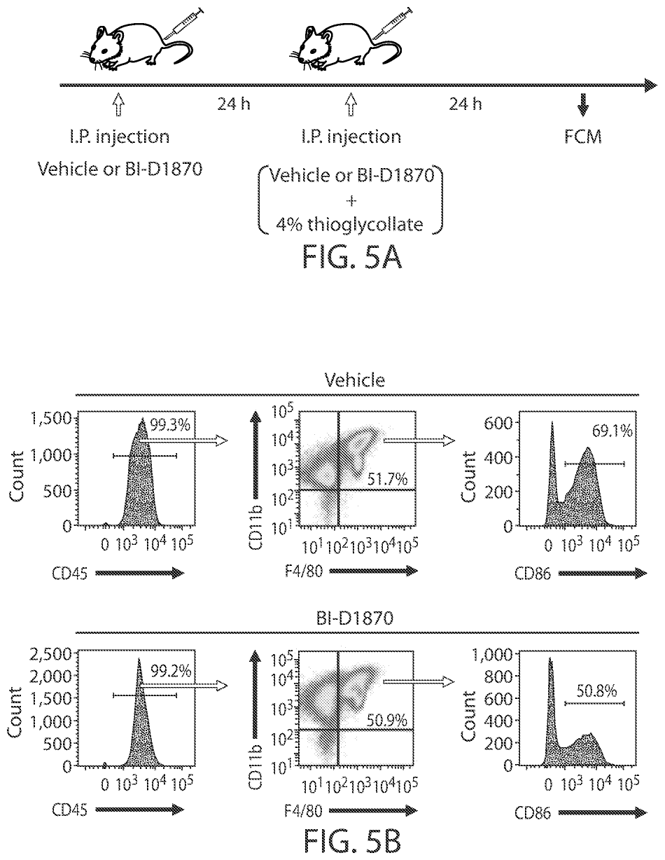

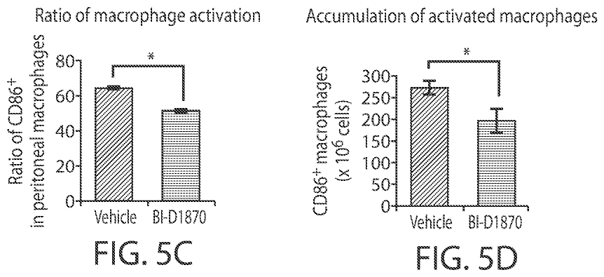

To determine whether RSK isoforms including RSK1 promote macrophage activation in vivo, we used a mouse model of thioglycollate-elicited peritonitis and BI-D1870. Mice were injected intraperitoneally with vehicle or 30 mg/kg BI-D1870 followed by thioglycollate-induced peritonitis (FIG. 5A). Twenty-four hours after the thioglycollate injection, we collected peritoneal cells from the mice and measured the population of activated macrophages (CD45⁺ CD11b⁺ F4/80⁺ CD86⁺ cells) using flow cytometry (FIGS. 5A and 5B). BI-D1870 suppressed the ratio of activated macrophages to total macrophages and the accumulation of activated macrophages in peritoneal cavity (FIGS. 5C and 5D).

Phospho-proteomics and network analysis link RSK-mediated phosphorylation to human inflammatory diseases.

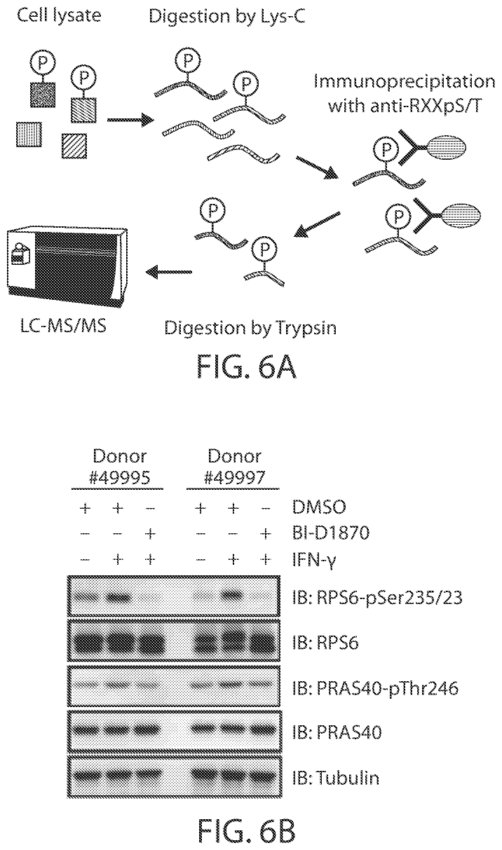

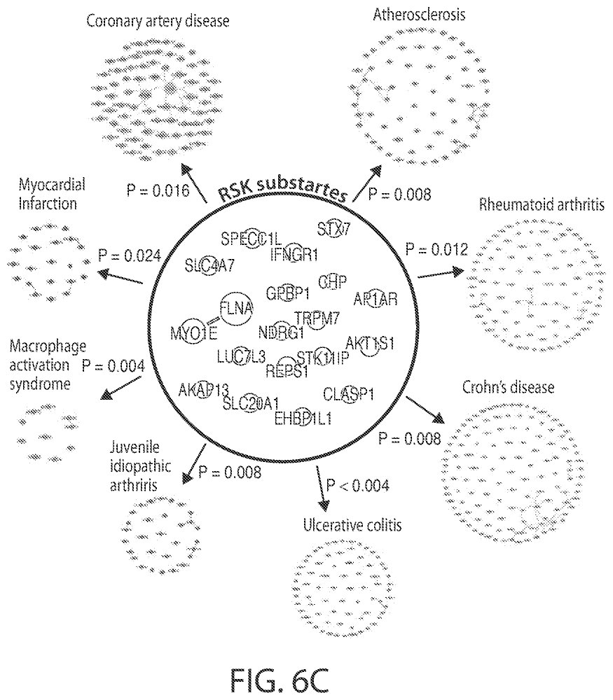

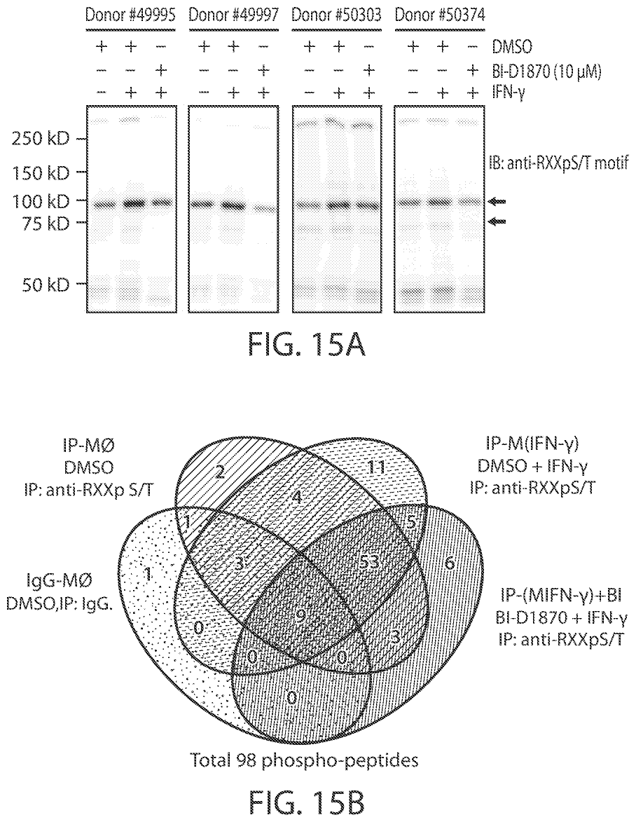

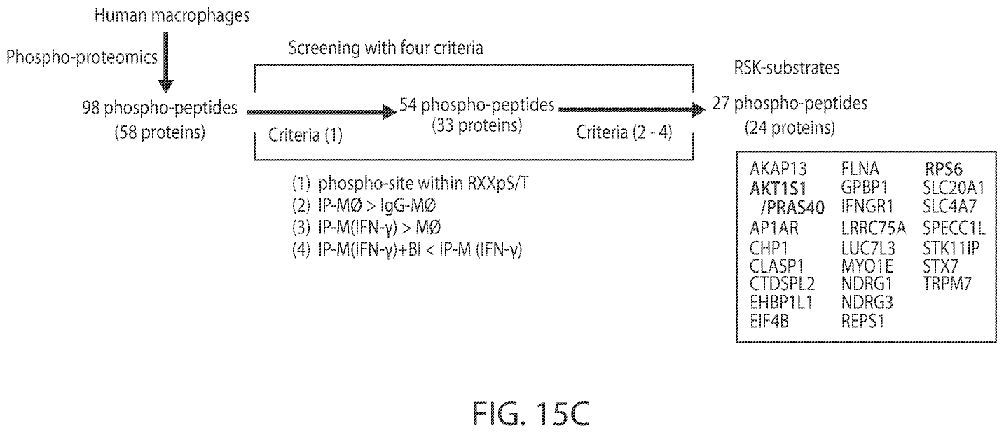

The RSK family of kinases prefer to phosphorylate serine or threonine in a consensus RXXS/T motif (FIG. 8), although these kinases are capable of phosphorylating sequences that differ from the consensus motif (e.g. STAT1 at LPMpS⁷²⁷ (FIG. 3), YB-1 at YLRpS¹⁰², RRN3/TIF-1A at MQPpS⁶⁴⁹, and ATP4 at PNR pS²⁴⁵ (35)). To explore phosphorylation signaling events via additional candidate RSK-substrates in proinflammatory activated macrophages, we focused on characterizing the global intracellular phosphorylation status of the commonly described RXXS/T consensus (FIG. 6A) (35). We thus performed phospho-proteomics using an anti-RXXpS/T antibody-based enrichment strategy (36). Human PBMC-derived macrophages from four donors were treated with DMSO or a pan-RSK inhibitor BI-D1870 followed by IFN-γ treatment. In order to achieve the minimum protein input (8.0 mg) for phospho-peptide enrichment, a pool of four donor macrophage cell lysates was needed. We therefore first verified whether the donors exhibited a similar phosphorylation pattern in response to IFN-γ treatment. Immunoblot analysis with the anti-RXXpS/T revealed a similar banding pattern in response to IFN-γ across the donors (FIG. 15A), and a similar decrease in signal intensities in response to in response to BI-D1870 treatment (FIG. 15A).

For the combined donor samples we performed four immunoprecipitation (IP) experiments: 1 and 2, unstimulated macrophages IPed with either IgG or anti-RXXpS/T; and 3 and 4, IFN-γ-stimulated macrophages plus/minus BI-D1870, IPed with anti-RXXpS/T (FIG. 15B). We identified 98 high-confident phospho-peptides corresponding to 58 proteins, of which 54 contained RXXpS/T motif (FIGS. 15 B and 15C). Approximately half of the phospho-peptides (53%) were common to all three anti-RXXpS/T IPs (FIG. 15B). Using a filtering strategy that removed non-specific hits as determined by the IgG control, we identified 24 IFN-γ induced phospho-proteins that decreased with BI-DI1870 treatment, including known RSK1-substrates, such as RPS6, a ribosomal protein S6 (37), and EIF4B, a eukaryotic translation initiation factor 4B (38) (FIG. 15C and Table 3). In addition, phospho-peptides derived from kinase-interacting proteins (AKT1S1/PRAS40, AKT1 substrate-1; AKAP13, A-kinase anchor protein 13; STK11IP, Serine/threonine-protein kinase 11-interacting protein) were also decreased with BI-D1870 treatment (FIG. 15C and Table 3), suggesting crosstalk of signal pathways between RSK kinases and other kinases (e.g. PKA/Protein kinase A and AKT/Protein kinase B) in pro-inflammatory activation of macrophages. To validate the changes in phosphorylation status between IFN-γ and RSK-inhibition conditions, we performed immunoblot analysis of RPS6-pSer235/236 and, for example, PRAS40-pThr246 using two of the four donors form the original phosphor-proteomics experiments, confirming the changes detected using mass spectrometry (FIG. 6B).

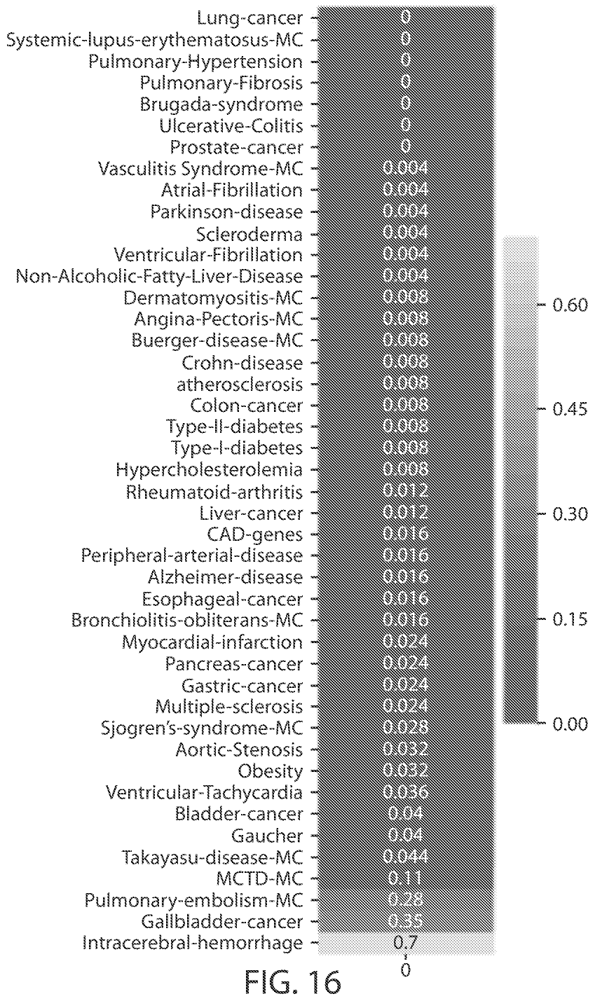

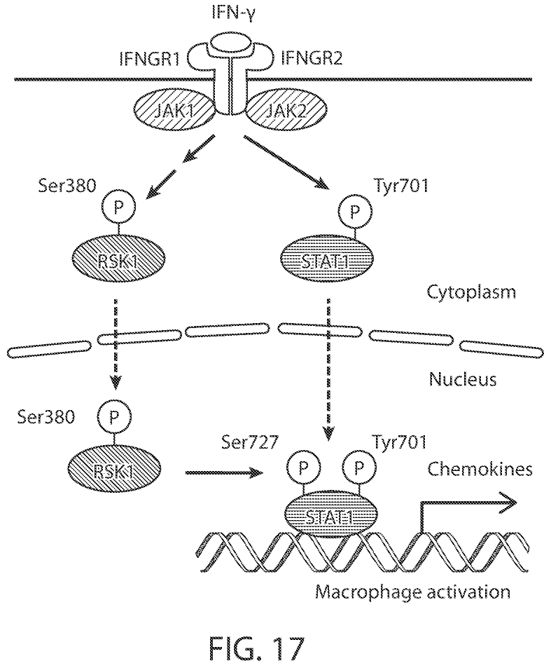

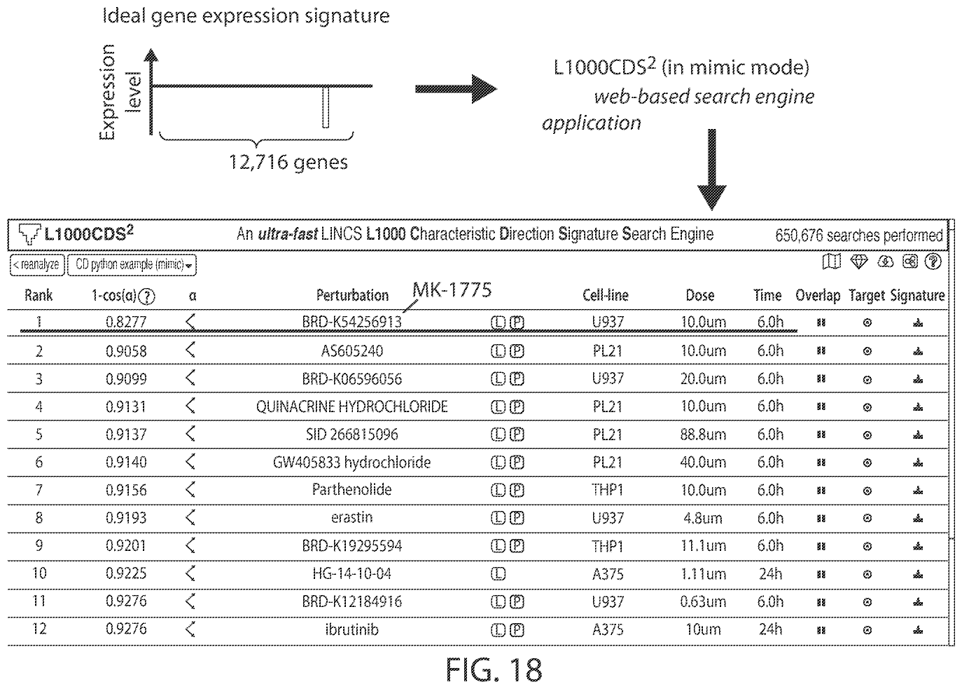

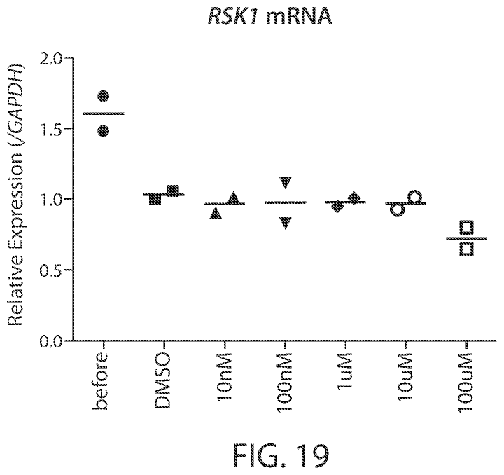

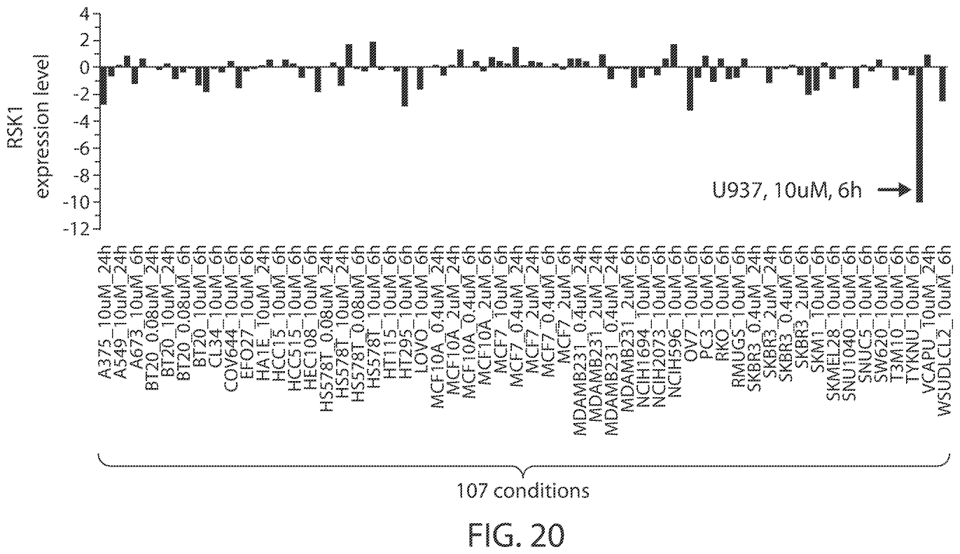

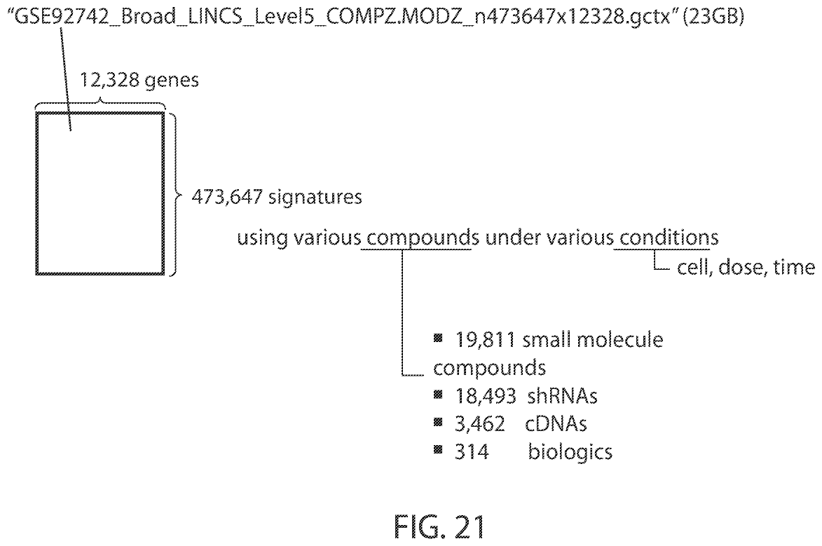

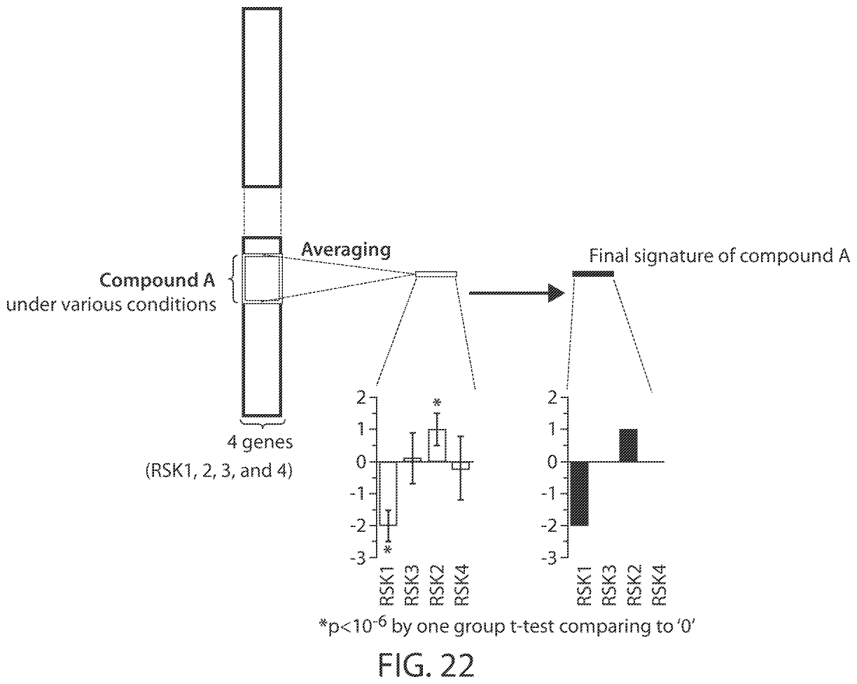

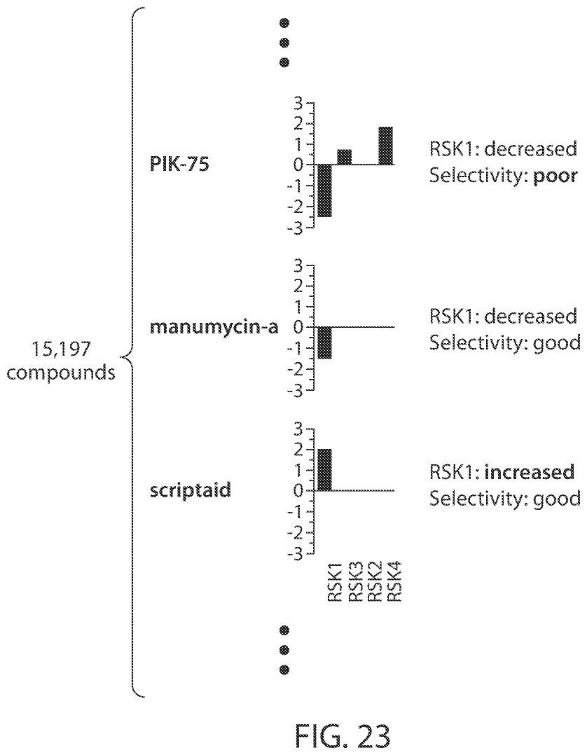

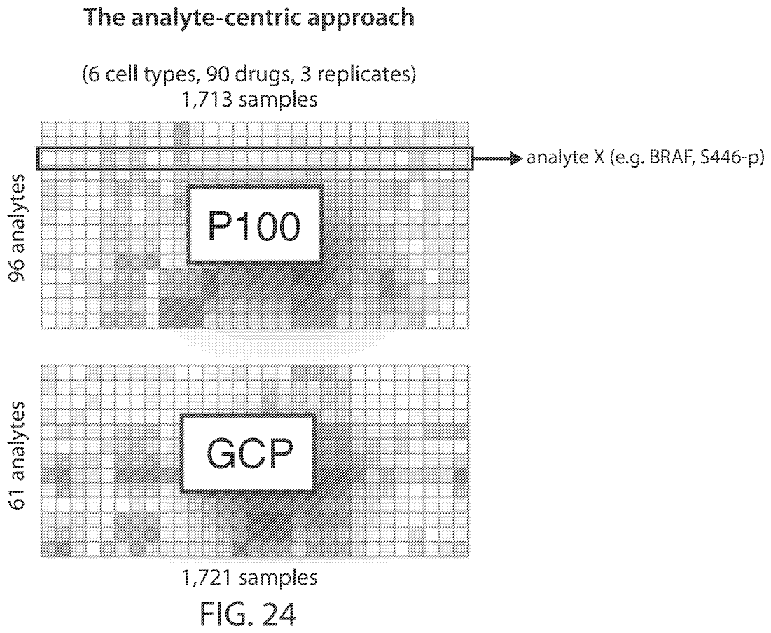

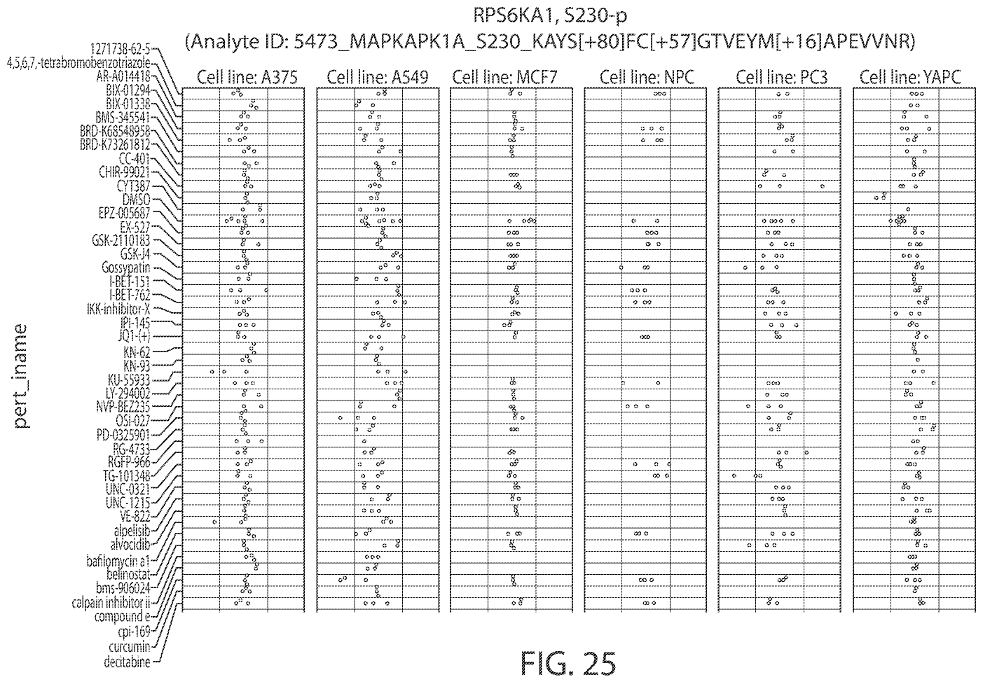

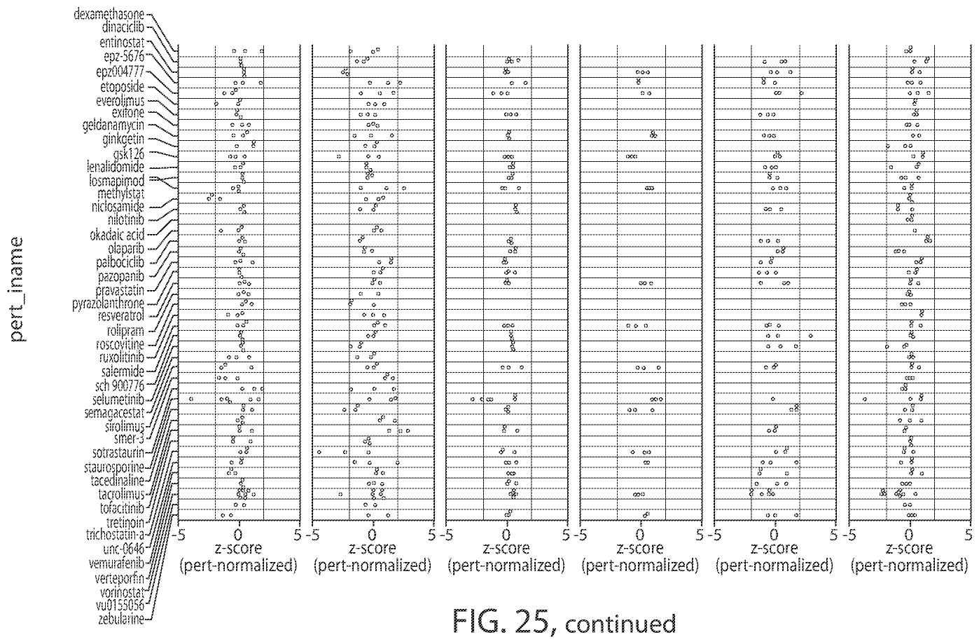

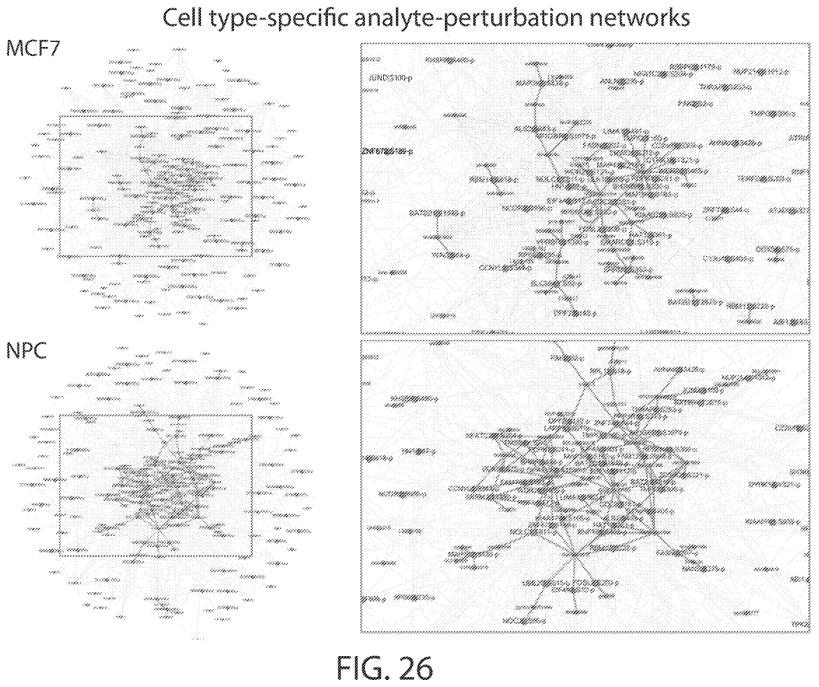

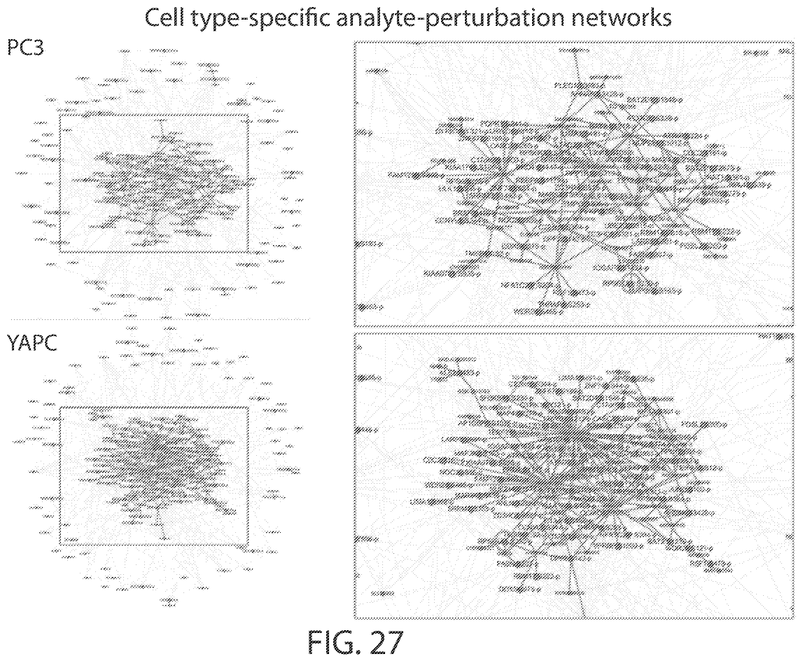

In addition to network proximity between the RSK1-first-neighbor and the human disease modules (FIG. 2), we further measured the network closeness between the RSK-substrate module and the human disease modules to screen for RSK-associated disorders. We identified that the RSK-substrates are significantly close to some of the tested inflammatory diseases, including autoimmune and cardiovascular diseases (FIG. 6C and FIG. 16). These results indicate that RSK-mediated phosphorylation of the substrates in pro-inflammatory activated macrophages links to human inflammatory diseases.

Discussion

This study demonstrates that RSK1 is a key nuclear shuttling enzyme that mediates IFN-γ-induced pro-inflammatory activation of human macrophages based on the following novel findings: 1) nine nuclear translocated protein candidates identified by human primary macrophage nuclear proteomics include RSK1; 2) RSK1 is closely associated with multiple human inflammatory diseases, as predicted by network analysis using the protein-protein interaction databases; 3) IFN-γ stimulation increases phospho-RSK1 Ser380 via JAK signaling in human primary macrophages; 4) RSK1 phosphorylation by IFN-γ leads to phosphorylation of STAT1 at Ser727; 6) RSK1 mediates IFN-γ-induced production of pro-inflammatory chemokines such as CCL2/MCP-1 by macrophages; 7) RSK suppression by the inhibitor BI-D1870 suppresses activation of peritoneal macrophages in mice; and 8) 22 proteins were identified as candidates of novel RSK-substrates in IFN-γ-stimulated macrophages by phospho-proteomics. These lines of evidence indicate a new theory that RSK1 functions as a key kinase of pro-inflammatory M(IFN-γ) macrophage activation mediated by the JAK-STAT pathway (see a schematic diagram in fig. S17).

We postulated that IFN-γ-induced nuclear translocation of regulators serves key roles for skewing macrophages to the M(IFN-γ) phenotype, based on several lines of evidence supporting that nucleocytoplasmic shuttling proteins control a variety of cellular responses in the nucleus (39-43). However, nuclear shuttling proteins that engage in pro-inflammatory macrophage activation have been largely unknown. In this study, we aimed to identify key nuclear shuttling enzyme(s) for M(IFN-γ) macrophage activation. To achieve this, we adopted mass spectrometry-based proteomics approach using nuclear lysates from human primary macrophages, because quantitative proteomics is suitable to monitor changes in the nuclei of macrophages during pro-inflammatory activation. As the results of screening, we identified RSK1 as a key nuclear shuttling enzyme for M(IFN-γ) macrophage activation.

The RSK serine/threonine kinase family is consist of four isoforms, RSK1, RSK2, RSK3, and RSK4, which regulate various cellular processes such as transcription, translation, cell cycle regulation, and cell survival (29, 35, 44). Although RSK isoforms show a high degree of sequence homology, increasing evidence supports functional differences among RSK isoforms, especially in cancer cells (35, 45). Three RSK isoforms, RSK1, RSK2, and RSK3, function as downstream effectors of the extracellular-signal-regulated kinase (ERK) signaling in response to mitogenic stimuli, whereas ERK signaling does not affect RSK4 due to its constitutive activation even in serum-starved cells (25, 37, 46). In several types of tumor cells, RSK1 and RSK2 contributes to tumor progression, invasion, and migration (47-49). Thus, RSK1 and RSK2 are considered as promising candidates of molecular targets for cancer therapies (44, 50). In contrast, RSK3 and RSK4 have been shown to act as tumor suppressors (51-53). Despite the biological importance of RSK isoforms, few studies have focused on additional RSK-mediated biological processes. In addition, the functionality of RSK-family members may depend on cell-types and contexts. Our findings highlight the new role of RSK1 in pro-inflammatory macrophage activation.

As with IFN-γ stimulation, RSK1 activation occurs in the cytoplasm and subsequently translocates to the nucleus upon exposure to EGF (35). Phosphorylation of RSK1 at Ser221 is essential for its nuclear targeting induced by EGF stimulation (54). Meanwhile, we showed that, in human primary macrophages, Ser221 was strongly phosphorylated without any stimulation (FIG. 10A). Given the finding, it is conceivable that IFN-γ-induced nuclear translocation of RSK1 is triggered by different types of molecular machinery, including other post-translational modification, which remains unclear. Hence further studies will need to address this question.

Our findings also indicate that RSK1 is responsible for IFN-γ-induced phosphorylation of STAT1 at Ser727 in human primary macrophages. Ser727 phosphorylation is essential for maximal activation of STAT1, contributing to IFN-γ-induced macrophage activation (30, 55). The S727A mutant in which Ser727 mutated to Ala differentially affects STAT1-target genes, s indicating that Ser727 phosphorylation also controls selectivity of STAT1 transactivation (55, 56). In this context, the Ser727 kinases could differentially promote STAT1-target genes as well as induce STAT1 activation in response to IFN-γ. Consistent with this notion, silencing RSK1 suppressed specific part of the STAT1-target genes including pro-inflammatory chemokines, e.g. CCL2, indicating that RSK1-mediated Ser727 phosphorylation selectively induces transactivation of STAT1-target genes in human macrophages (FIG. 4, FIG. 13). In addition, IFN-γ-induced Ser727 phosphorylation requires nuclear translocation of STAT1 (12), indicating that the Ser727 kinase phosphorylates STAT1 in the nucleus. Concerning our finding that RSK1 translocates to the nucleus in response to IFN-γ, RSK1 appears to phosphorylate STAT1 at Ser727 after their nuclear translocation in pro-inflammatory activated macrophages.

In summary, we demonstrate that RSK1 plays a key kinase that translocates to the nucleus for shifting human primary macrophages toward pro-inflammatory phenotype. We also present a novel mechanism by which RSK1 controls transcriptional activity and target selectivity of STAT1 through Ser727 phosphorylation to promote secretion of pro-inflammatory chemokines in IFN-γ-stimulated macrophages. This study provides new insight into molecular basis for RSK1-mediated pro-inflammatory activation of macrophages, which is the first step toward the design of an effective therapy for patients with macrophage-mediated inflammatory diseases.

### Example 2

### Material and Methods

Cell culture of human PBMC-derived primary macrophages. Human PBMCs were isolated from buffy coat using lymphocyte separation medium (MP Biomedicals) according to the instructions of the manufacturer. PBMCs were incubated in RPMI-1640 without serum for one hour, washed with Hanks' Balanced Salt Solution, and cultured in RPMI-1640 containing 5% human serum (Gemini Bio-Products), penicillin, and streptomycin. After differentiation for ten days, we used the cells as human PBMC-derived macrophages. Cells were maintained at 37° C. in 5% CO². Cells were treated with IFN-γ (R&D Systems), DMSO (Sigma-Aldrich), BI-D1870 (RSK Inhibitor II; EMD Millipore), or pyridone-6 (JAK Inhibitor I; EMD Millipore).

Subcellular fractionation. We used ProteoExtract Subcellular Proteome Extraction Kit (EMD Millipore) to obtain nuclear lysates of human macrophages in according to the instructions of the manufacturer. Purity of the fractions was monitored using immunoblot analysis (see below).

Tandem mass tagging (TMT) sample preparation. We stimulated human PBMC-derived macrophages obtained from three donors (donor A, #44383; donor B, #44442; donor C, #44400) with IFN-γ for 0, 10, 20, 30, or 60 minutes. Nuclear fractions of each condition were isolated using ProteoExtract Subcellular Proteome Extraction Kit (EMD Millipore) and proteolysed (Lys-C, Wako Chemicals) using in-solution urea strategy detailed previously (34). Peptides were labeled with TMT 10-plex reagent (Pierce). The reporter ion channels were assigned for two sets of running as follows: for first running, 126 (0 minutes with IFN-γ, donor A), 127N (10 minutes with IFN-γ, donor A), 128N (20 minutes with IFN-γ, donor A), 129N (30 minutes with IFN-γ, donor A), 130N (60 minutes with IFN-γ, donor A), 127C (60 minutes with IFN-γ, donor B), 128C (30 minutes with IFN-γ, donor B), 129C (20 minutes with IFN-γ, donor B), 130C (10 minutes with IFN-γ, donor B) and 131 (0 minutes with IFN-γ, donor B); for second running, 126 (0 minutes with IFN-γ, donor A), 127N (10 minutes with IFN-γ, donor A), 128N (20 minutes with IFN-γ, donor A), 129N (30 minutes with IFN-γ, donor A), 130N (60 minutes with IFN-γ, donor A), 127C (60 minutes with IFN-γ, donor C), 128C (30 minutes with IFN-γ, donor C), 129C (20 minutes with IFN-γ, donor C), 130C (10 minutes with IFN-γ, donor C) and 131 (0 minutes with IFN-γ, donor C). The labeled peptides were combined and desalted using Oasis Hlb 1 cc columns (Waters). The peptides were then fractionated into 24 fractions based on their isoelectric focusing point (pH range of 3-10) using the OFF-gel system (Agilent). The fractions were dried using a tabletop speed vacuum, cleaned with the Oasis columns and resuspended in 40 μl of 5% acetonitrile and 0.5% formic acid for subsequent analysis by liquid chromatography/mass spectrometry (LC/MS).

Phospho-proteomics. Phospho-peptide immunoaffinity purification from cell lysates was performed as described previously (36), with minor modifications. Human PBMC-derived macrophages were pretreated with DMSO or BI-D1870 followed by IFN-γ stimulation. Cell lysates (8.0 mg) were proteolyzed (Lys-C, Wako Chemicals) using in-solution urea strategy detailed previously (34). Trifluoroacetic acid (TFA) was added to protein digests to a final concentration of 1%, precipitate was removed by centrifugation, and digests were loaded onto Sep-Pak C18 columns (Waters) equilibrated with 0.1% TFA. Columns were washed with 0.1% TFA and wash buffer (0.1% TFA, 5% acetonitrile). A peptide fraction was obtained by elution with elution buffer (0.1% TFA, 40% acetonitrile). The peptide eluate was frozen overnight and lyophilize frozen peptide solution for 2 days. Peptides were dissolved in 1.4 mL of IAP buffer (Cell signaling Technology). Insoluble matter was removed by centrifugation. Phospho-Akt Substrate (RXXS*/T*) (110B7E) Rabbit mAb (Sepharose Bead Conjugate) (#9646; Cell Signaling Technology) washed with PBS was added to the peptide solution and incubated at 4° C. for two hours. The immobilized antibody beads were washed three times with 1 ml IAP buffer and three times with 1 ml water, all at 4° C. Peptides were eluted from beads by incubation with 55 μl of 0.15% TFA at room temperature for 10 minutes (eluate 1), followed by a wash of the beads (eluate 2) with 50 μl of 0.15% TFA. Both eluates were combined. The peptide solution was desalted using Oasis Hlb 1 cc columns (Waters), dried in a SpeedVac, resuspend with Trypsin solution, and digested overnight. After desalting using Oasis Hlb 1 cc columns, peptides were dried in a SpeedVac, and resuspend with 40 μl of 5% acetonitrile and 0.5% formic acid for subsequent analysis by liquid chromatography/mass spectrometry (LC/MS). To identify RSK-substrates which possess phosphorylation within the RXXS*/T* motif, we filtered based on four criteria; (1) a detected phosphorylation site is found in the motif, (2) a ratio of signal intensity of IP with anti-RXXS*/T* motif over that of IP with control IgG is higher than 1.00, (3) a ratio of signal intensity of IFN-γ-stimulated cells over that of unstimulated cells is higher than 1.00, (4) a ratio of signal intensity of IFN-γ plus BI-D1870 over IFN-γ minus BI-D1870 is higher than 1.00.

Liquid chromatography tandem mass spectrometry (LC-MS/MS). TMT studies—The high resolution/accuracy Q Exactive mass spectrometer fronted with a Nanospray FLEX ion source, coupled to an Easy-nLC1000 HPLC pump (Thermo Scientific) was used to analyze the TMT peptide samples. The analytical gradient was run at 300 nl/minutes from 5 to 18% Solvent B (acetonitrile/0.1% formic acid) for 120 minutes, followed by five minutes of 95% Solvent B. Solvent A was 0.1% formic acid. The precursor scan was set to 140 K resolution, and the top 10 precursor ions (within a scan range of 380-2000 m/z) were subjected to higher energy collision induced dissociation (HCD, collision energy 30%, isolation width 3.0 m/z, dynamic exclusion enabled, starting m/z fixed at 120 m/z, and resolution set to 35 K) for peptide sequencing (MS/MS).

Phosphoproteomics—Phospho-peptides were analyzed on the Orbitrap Fusion Lumos (with Easy-Spray ion source and Easy-nLC1000 HPLC pump), using electron-transfer/higher-energy collision dissociation (EThcD) for phopsho-peptide sequencing. The gradient flow rate was 300 nl/min from 5 to 21% solvent B (acetonitrile/0.1% formic acid) for 80 minutes, 21 to 30% solvent B for ten minutes, followed by five minutes of 95% solvent B. Solvent A was 0.1% formic acid. Each peptide sample was analyzed four times: a full scan range of 350-1800 m/z and three gas phase separation scans—350-500 m/z, 500-700 m/z, and 700-1200 m/z in order to increase phospho-peptide signals. The MS/MS were acquired as follows: calibrated charge dependent ETD parameters enabled, HCD collision energy 30%, and resolution set to 60 K. The peptides that had higher charge state and lower m/z were prioritized for MS/MS.

Anti-STAT1-pSer727 IPs—The STAT1 peptide with phosphorylation at Ser727 was detected on the Orbitrap Fusion Lumos. The gradient flow rate was 300 nL/min from 5 to 21% solvent B (acetonitrile/0.1% formic acid) for 80 minutes, 21 to 30% solvent B for ten minutes, followed by five minutes of 95% solvent B. Solvent A was 0.1% formic acid. The target phosphorylated STAT1 peptide, LQTTDNLLPmsPEEFDEVSR (m10-oxidation, s11-phosphorylation, 806.3575 m/z, z=3), was subjected to EThcD (calibrated charge dependent ETD parameters enabled, HCD collision energy 30%, and resolution set to 500 k) for MS/MS.

LC-MS/MS data analysis.TMT studies—The MS/MS data were queried against the human UniProt database (downloaded on Aug. 1, 2014) using the SEQUEST search algorithm, via the Proteome Discoverer version 2.1 (PD2.1, Thermo Scientific), using a 10-ppm tolerance window in the MS1 search space, and a 0.02 Da fragment tolerance window for HCD. Methionine oxidation was set as a variable modification, and carbamidomethylation of cysteine residues and 10-plex TMT tags (Thermo Scientific) were set as fixed modifications. The peptide false discovery rate (FDR) was calculated using Percolator provided by PD: the FDR was determined based on the number of MS/MS spectral hits when searched against the reverse, decoy human database. Peptides were filtered based on a 1% FDR. Peptides assigned to a given protein group, and not present in any other protein group, were considered as unique. Consequently, each protein group is represented by a single master protein (PD Grouping feature). Master proteins with two or more unique peptides were used for TMT reporter ratio quantification. The normalized reporter ion intensities were exported from PD2.1 the analysis below.

Phospho-proteomics and kinase assays. The MS/MS data were queried as above using a 10-ppm tolerance window in the MS1 search space, and a 0.02 Da fragment tolerance window for EThcD or HCD. Methionine oxidation, and phosphorylation of serine and threonine were set as variable modifications, and carbamidomethylation of cysteine residues was set as fixed modifications. High confidently assigned phospho-peptides were used for precursor ion area under the curve (AUC) quantification. The peptides that were detected in IgG conditions were considered as non-specific signals and excluded. The normalized precursor ion intensities were exported from PD2.1.

Anti-STAT1-pSer727 IPs—The three most abundant fragment ions of the target peptide (LQTTDNLLPmsPEEFDEVSR (SEQ ID NO: 5)-m10-oxidation, s11-phosphorylation, 806.3575 m/z, z=3) as annotated by SEQUEST (PD2.1) were used for quantification: y12²⁺, 759.79455 m/z; c12⁺, 1424.64911 m/z; b8⁺, 899.48327 m/z. AUC of each fragment was calculated using the Skyline software (https://skyline.gs.washington.edu).

Multiplexed cluster analysis. High-dimensional clustering of the normalized TMT ion intensities was done using our published software, XINA (19). Our method is unique from standard clustering approaches in that we combine the kinetics data acquired from multiple datasets (e.g., the two TMT 10-plex experiments) into a single input file for clustering, under the assumption that the sources and extent of variation (response to IFN-γ) across the experiments, the three donors' kinetics, are similar (19). The value in this multiplexing approach includes a simplified output of a single set of clusters and the ability to monitor the behavior of a single protein across various conditions (in this case, the three donors' nuclear responses to IFN-γ). In this study, we combined the three independent nuclear translocation datasets: The five timepoint kinetics of Donor A (the average the two TMT 10-plex replicate data), Donor B and Donor C for subsequent clustering. We ran model-based clustering analysis using ‘mclust’ R package, resulting in a 41-clusters that explain the variation (donor and IFN-γ response) in the combined data (FIG. 7A).

Immunoblot analysis and immunoprecipitation. Cells were harvested, washed with phosphate-buffered saline (PBS), and suspended with Lysis buffer (20 mM Tris-HCl, pH 7.5; 150 mM NaCl; 1 mM Na₂EDTA; 1 mM EGTA; 1% Triton; 2.5 mM sodium pyrophosphate; 1 mM beta-glycerophosphate; 1 mM Na₃VO₄; 1 μs/ml leupeptin) containing protease inhibitors (Roche). After centrifugation, the supernatants were isolated and used as whole cell lysates. For immunoprecipitation, cell lysates were incubated with normal IgG or anti-RSK1 for two hours followed by incubation with Protein A agarose beads (Cell Signal Technology) for one hour at 4° C. The beads were washed with lysis buffer and re-suspended with lysis buffer. Whole cell lysates and subcellular fractions, and immunoprecipitated proteins were boiled with sample buffer for five minutes, separated by SDS-PAGE, transferred onto nitrocellulose membranes. The membranes were blocked with 2.5% skim milk in TBS with 0.05% Tween 20 (TBS-T) and incubated with anti-RSK1 (#sc-231; Santa Cruz Biotechnology), anti-RSK1 (#8408; Cell Signal Technology), anti-RSK2 (#sc-9986; Santa Cruz Biotechnology), anti-RSK3 (#sc-1431; Santa Cruz Biotechnology), anti-RSK4 (sc-100424; Santa Cruz Biotechnology), anti-STAT1 (#610115; BD Biosciences), anti-Lamin A/C (#39287, Active Motif), anti-phospho-RSK1-Ser221 (#AF892; R&D systems), anti-phospho-RSK1-Thr359 (#8753; Cell Signaling Technology), anti-phospho-RSK1-Ser380 (#11989; Cell Signaling Technology), anti-phospho-RSK1-Thr573 (#9346; Cell Signaling Technology), anti-phospho-RSK1-Ser732 (#600-401-B30S; Rockland), anti-phospho-STAT1-Ser727 (#8826; Cell Signaling Technology), anti-phospho-STAT1-Tyr701 (#9167; Cell Signaling Technology), anti-Tubulin (#T5168; Sigma-Ardrich), anti-phospho-RPS6-Ser235/236 (#A300-584A; Bethyl Laboratories), anti-RPS6 (#A300-556A; Bethyl Laboratories), anti-phospho-PRAS40-Thr246 (#13175; Cell Signaling Technology), or anti-PRAS40 (#2691; Cell Signaling Technology). Membranes were then washed with TBS-T, incubated with peroxidase-conjugated anti-rabbit IgG (Fisher Scientific) or peroxidase-conjugated anti-mouse IgG (Fisher Scientific), and washed with TBS-T. Immune complexes were visualized using SuperSignal West Dura Extended Duration Substrate (Thermo Fisher Scientific). Digital image data was obtained with ImageQuant Las 4000 (GE Healthcare).

SYPRO Ruby staining and in-gel proteolysis. We performed gel staining using SYPRO Ruby Protein Gel Stain (Thermo Fisher Science) according to the instructions supplied by the manufacturer. After SDS-PAGE, the gels were placed in fix solution (50% methanol, 7% acetic acid) for 30 minutes twice. The gels were stained with SYPRO Ruby Gel Stain overnight. The gels were incubated with wash solution (10% methanol, 7% acetic acid) for 30 minutes, followed by rinse with water three times for five minutes. Digital images of the stained gels were obtained using ImageQuant Las 4000. The prominent band corresponding to the expected molecular weight for STAT1 was excised for in-gel trypsinization (57). Peptides were dissolved in sample loading buffer (0.1% formic acid, 5% acetonitrile) for subsequent mass spectrometric analysis.

Immunofluorescence assays. Cells cultured in chamber slides were fixed in 4% paraformaldehyde for 15 minutes, permeabilized in 0.5% Triton X-100 for 15 minutes, washed with phosphate-buffered saline (PBS), and blocked with 10% goat serum in PBS saline for 30 minutes. After washing with PBS, the cells were immuno-stained with anti-RSK1 (#sc-231; Santa Cruz) followed by reaction with Alexa Fluor 498-conjugated secondary antibodies. Nuclei were stained with 4,6-diamidino-2-phenylindole (Vector Laboratories). Images were obtained with Eclipse 80i fluorescent microscope (Nikon).

Network analysis. As a proxy of the association between the RSK family of proteins and human diseases, the average shortest network distance between RSK modules and disease-related proteins were measured, where network distance is defined as the non-Euclidean distance measured in terms of the number of edges between two nodes. RSK modules are defined as the subgraphs consisting of the RSK family gene and its first neighbors, i.e. direct interaction partners on the interactome. The average shortest distance D of an RSK module to disease genes is measured by calculating the shortest distance between each RSK module gene s and all genes t of a disease and then averaging over all RSK module genes s such that and, where is the shortest distance between s and t and S and T are the sets of genes in the RSK first neighbors module and disease genes, respectively. To compare the average shortest distance value to random expectation, the average shortest distance of the same number of randomly selected genes to disease genes was calculated for N=100 realizations. To control for degree (i.e., the number of connections of a gene), the random selection was done in a degree-preserving manner where all genes were binned according to their degree and random genes were selected uniformly at random from their corresponding degree bin. Empirical p-values were calculated by, where is the average shortest distance of the randomized instance. The interactome onto which the RSK modules and disease genes were mapped consists of curated physical protein-protein interactions with experimental support, including binary interactions, protein complexes, enzyme-coupled reactions, signaling interactions, kinase-substrate pairs, regulatory interactions and manually curated interactions from literature, as described previously (28). Disease genes were obtained from the DiseaseConnect (available on the world wide web at http://disease-connect.org) (18) (using entries with evidence from Genome-Wide Association Studies (GWAS) and Online Mendelian Inheritance in Man (OMIM) (available on the world wide web at www.omim.org/)) and MalaCards (available on the world wide web at www.malacards.org/) (58) databases. Cellular localizations of proteins were assessed using the Uniprot database (accessed Feb. 20, 2018) using the Subcellular Location annotations. For the identification of nuclear proteins, “Chromosome, centromere, kinetochore”, “Nucleus” and “Nucleus” speckle were considered as nuclear locations.

Cell transfections. Transfections of macrophages with siRNA were performed using SilenceMag (BOCA Scientific) according to the instructions of the manufacturer. Target sequences of siRNA are follows:

For non-targeting control pool:
(SEQ ID NO: 6)
5′-UGGUUUACAUGUCGACUAA-3′,
(SEQ ID NO: 7)
5′-UGGUUUACAUGUUGUGUGA-3′,
(SEQ ID NO: 8)
5′-UGGUUUACAUGUUUUCUGA-3′,
and
(SEQ ID NO: 9)
5′-UGGUUUACAUGUUUUCCUA-3′.
For human RSK1/RPS6KA1 pool:
(SEQ ID NO: 10)
5′-GUGGGCACCUGUAUGCUAU-3′,
(SEQ ID NO: 11)
5′-GAUAAGAGCAAGCGGGAUC-3′,
(SEQ ID NO: 12)
5′-GAAAGUACGUGACCGCGUC-3′,
and
(SEQ ID NO: 13)
5′-GAACACAGUUUCAGAGACA-3′.

Real-time PCR. Total RNA from cells was isolated using TRIzol (Thermo Fisher Scientific) according to the instructions of the manufacturer. Reverse transcription was performed using qScript cDNA Synthesis Kits (QuantaBio). The mRNA levels were determined by TaqMan-based real-time PCR reactions (Thermo Fisher Scientific). The following TaqMan probes were used: human RSK1/RPS6KA1 (Hs01546654_m1), human RSK2/RPS6KA3 (Hs00177936_m1), human RSK3/RPS6KA2 (Hs00179731_m1), human CCL2 (Hs00234140_m1), human CCL7 (Hs00171147_m1), human CCL8 (Hs04187715_m1), human CXCL9 (Hs00171065_m1), human CXCL10 (Hs01124251_g1), human CXCL11 (Hs04187682_g1), human STAT1 (Hs01013996_m1), human IRF1 (Hs00971960_m1), human PARP14 (Hs00981511_m1), human PARP9 (Hs00967084_m1), human GBP1 (Hs00977005_m1), human TAP1 (Hs00388677_m1), human FCGR1B (Hs00417598_m1), human GAPDH (Hs02758991_g1). Data were normalized by human GAPDH and then calculated using the delta-delta Ct method.

ELISA. The amounts of human CCL2/MCP-1, human CCL7/MCP-3, human CCL8/MCP-2, human CXCL9/MIG, human CXCL10/IP-10, and human CXCL11/I-TAC proteins in the culture media were measured using DUOSET ELISA kits (R&D Systems) according to the manufacturer's instruction.

Mouse peritonitis model. C57BL/6J wild type mice (12 weeks old, male) were purchased from Jackson Laboratory. We injected intraperitoneally with vehicle (30% PEG400, 0.5% Tween80, 5% Propylene glycol) or 30 mg/kg BI-D1870 (Selleck Chemicals). After 24 hours, we injected intraperitoneally with 0.5 ml of 4% thioglycollate (Fisher Scientific), as well as vehicle or 30 mg/kg BI-D1870. Twenty-four hours after thioglycollate-injection, peritoneal cells were collected from the peritoneal cavity. All animal procedures used in this study were approved by and performed in compliance with Beth Israel Deaconess Medical Center's Institutional Animal Care and Use Committee.

Flow cytometry. Peritoneal cells from mice were incubated with anti-CD16/CD32 (#101319, BioLegend) to block the Fc receptor. Cells were then stained with anti-CD45-allophycocyanin (APC)/Cy7 (#103116, BioLegend), anti-CD11b-APC (#101212, BioLegend), anti-Ly-6G-phycoerythrin (PE) (#127608, BioLegend), anti-CD86-PE/Cy7 (#105116, BioLegend), anti-F4/80-fluorescein isothiocyanate (FITC) (#122606, BioLegend) in EasySep Buffer (STEMCELL Technologies) for 30 minutes. After washing cells with EasySep Buffer, stained cells were analyzed by BD FACSAria II (BD Bioscience) and FlowJo software (FlowJo LLC).

### REFERENCES

1. K. J. Moore, I. Tabas, Macrophages in the pathogenesis of atherosclerosis. Cell 145, 341-355 (2011).
2. M. Aikawa, P. Libby, The vulnerable atherosclerotic plaque: pathogenesis and therapeutic approach. Cardiovascular pathology: the official journal of the Society for Cardiovascular Pathology 13, 125-138 (2004).
3. G. Chinetti-Gbaguidi, S. Colin, B. Staels, Macrophage subsets in atherosclerosis. Nature Reviews Cardiology 12, 10-17 (2015).
4. K. J. Moore, F. J. Sheedy, E. A. Fisher, Macrophages in atherosclerosis: a dynamic balance. Nat Rev Immunol 13, 709-721 (2013).
5. J. Xue et al., Transcriptome-based network analysis reveals a spectrum model of human macrophage activation. Immunity 40, 274-288 (2014).
6. M. Becker et al., Integrated Transcriptomics Establish Macrophage Polarization Signatures and have Potential Applications for Clinical Health and Disease. Sci Rep 5, 13351 (2015).
7. F. O. Martinez, S. Gordon, The M1 and M2 paradigm of macrophage activation: time for reassessment. F1000prime reports 6, 13 (2014).
8. P. J. Murray et al., Macrophage activation and polarization: nomenclature and experimental guidelines. Immunity 41, 14-20 (2014).
9. I. Tabas, K. E. Bornfeldt, Macrophage Phenotype and Function in Different Stages of Atherosclerosis. Circ Res 118, 653-667 (2016).
10. L. K. Kozicky, L. M. Sly, Phosphatase regulation of macrophage activation. Seminars in Immunology 27, 276-285 (2015).
11. K. Shuai, G. Stark, I. Kerr, J. Darnell, A single phosphotyrosine residue of Stat91 required for gene activation by interferon-gamma. Science 261, 1744-1746 (1993).
12. I. Sadzak et al., Recruitment of Stat1 to chromatin is required for interferon-induced serine phosphorylation of Stat1 transactivation domain. Proc Natl Acad Sci USA 105, 8944-8949 (2008).
13. X. Hu, K.-H. Park-Min, H. H. Ho, L. B. Ivashkiv, IFN-γ-Primed Macrophages Exhibit Increased CCR2-Dependent Migration and Altered IFN-γ Responses Mediated by Stat1. The Journal of Immunology 175, 3637-3647 (2005).
14. P. S. Subramaniam, J. Larkin, 3rd, M. G. Mujtaba, M. R. Walter, H. M. Johnson, The COOH-terminal nuclear localization sequence of interferon gamma regulates STAT1 alpha nuclear translocation at an intracellular site. J Cell Sci 113 (Pt 15), 2771-2781 (2000).
15. R. L. Haspel, M. Salditt-Georgieff, J. E. Darnell, The rapid inactivation of nuclear tyrosine phosphorylated Stat1 depends upon a protein tyrosine phosphatase. The EMBO Journal 15, 6262-6268 (1996).
16. UniProt: a hub for protein information. Nucleic Acids Res 43, D204-212 (2015).
17. M. Uhlen et al., Proteomics. Tissue-based map of the human proteome. Science 347, 1260419 (2015).
18. J. X. Binder et al., COMPARTMENTS: unification and visualization of protein subcellular localization evidence. Database 2014, bau012 (2014).
19. L. H. Lee et al., XINA: A Workflow for the Integration of Multiplexed Proteomics Kinetics Data with Network Analysis. J Proteome Res, (2018).
20. P. Ricchiuto et al., mIMT-visHTS: A novel method for multiplexing isobaric mass tagged datasets with an accompanying visualization high throughput screening tool for protein profiling. Journal of proteomics 128, 132-140 (2015).
21. R. Chen, C. Sarnecki, J. Blenis, Nuclear localization and regulation of erk-and rsk-encoded protein kinases. Molecular and cellular biology 12, 915-927 (1992).
22. T. Nakajima et al., The Signal-Dependent Coactivator CBP Is a Nuclear Target for pp90RSK. Cell 86, 465-474 (1996).
23. D. De Cesare, S. Jacquot, A. Hanauer, P. Sassone-Corsi, Rsk-2 activity is necessary for epidermal growth factor-induced phosphorylation of CREB protein and transcription of c-fos gene. Proc Natl Acad Sci USA 95, 12202-12207 (1998).
24. Y. Zhao, C. Bjørbaek, S. Weremowicz, C. C. Morton, D. E. Moller, RSK3 encodes a novel pp90rsk isoform with a unique N-terminal sequence: growth factor-stimulated kinase function and nuclear translocation. Molecular and Cellular Biology 15, 4353-4363 (1995).
25. B. A. Dummler et al., Functional characterization of human RSK4, a new 90-kDa ribosomal S6 kinase, reveals constitutive activation in most cell types. J Biol Chem 280, 13304-13314 (2005).
26. A.-L. Barabási, N. Gulbahce, J. Loscalzo, Network Medicine: A Network-based Approach to Human Disease. Nature reviews. Genetics 12, 56-68 (2011).
27. J. Loscalzo, A.-L. Barabási, E. K. Silverman, Network Medicine: Complex Systems in Human Disease and Therapeutics. (Harvard University Press, 2016).
28. J. Menche et al., Disease networks. Uncovering disease-disease relationships through the incomplete interactome. Science 347, 1257601 (2015).
29. R. Anjum, J. Blenis, The RSK family of kinases: emerging roles in cellular signalling. Nat Rev Mol Cell Biol 9, 747-758 (2008).
30. Z. Wen, Z. Zhong, J. E. Darnell, Jr., Maximal activation of transcription by Stat1 and Stat3 requires both tyrosine and serine phosphorylation. Cell 82, 241-250 (1995).
31. G. P. Sapkota et al., BI-D1870 is a specific inhibitor of the p90 RSK (ribosomal S6 kinase) isoforms in vitro and in vivo. Biochem J 401, 29-38 (2007).
32. J. Satoh, H. Tabunoki, A Comprehensive Profile of ChIP-Seq-Based STAT1 Target Genes Suggests the Complexity of STAT1-Mediated Gene Regulatory Mechanisms. Gene regulation and systems biology 7, 41-56 (2013).
33. M. A. Meraz et al., Targeted disruption of the Stat1 gene in mice reveals unexpected physiologic specificity in the JAK-STAT signaling pathway. Cell 84, 431-442 (1996).
34. H. Iwata et al., PARP9 and PARP14 cross-regulate macrophage activation via STAT1 ADP-ribosylation. Nat Commun 7, 12849 (2016).
35. Y. Romeo, X. Zhang, P. P. Roux, Regulation and function of the RSK family of protein kinases. Biochem J 441, 553-569 (2012).
36. A. Moritz et al., Akt-RSK-S6-kinase Signaling Networks Activated by Oncogenic Receptor Tyrosine Kinases. Science signaling 3, ra64-ra64 (2010).
37. P. P. Roux et al., RAS/ERK Signaling Promotes Site-specific Ribosomal Protein S6 Phosphorylation via RSK and Stimulates Cap-dependent Translation. Journal of Biological Chemistry 282, 14056-14064 (2007).
38. E. Kuang, B. Fu, Q. Liang, J. Myoung, F. Zhu, Phosphorylation of Eukaryotic Translation Initiation Factor 4B (EIF4B) by Open Reading Frame 45/p90 Ribosomal S6 Kinase (ORF45/RSK) Signaling Axis Facilitates Protein Translation during Kaposi Sarcoma-associated Herpesvirus (KSHV) Lytic Replication. The Journal of Biological Chemistry 286, 41171-41182 (2011).
39. L. Xu, J. Massague, Nucleocytoplasmic shuttling of signal transducers. Nat Rev Mol Cell Biol 5, 209-219 (2004).
40. F. Conforti et al., Molecular Pathways: Anticancer Activity by Inhibition of Nucleocytoplasmic Shuttling. Clinical Cancer Research 21, 4508-4513 (2015).
41. S. Yoon, K. Bogdanov, A. Kovalenko, D. Wallach, Necroptosis is preceded by nuclear translocation of the signaling proteins that induce it. Cell Death and Differentiation 23, 253-260 (2016).
42. K. Nihira, N. Taira, Y. Miki, K. Yoshida, TTK/Mps1 controls nuclear targeting of c-Abl by 14-3-3-coupled phosphorylation in response to oxidative stress. Oncogene 27, 7285-7295 (2008).
43. N. Taira, K. Nihira, T. Yamaguchi, Y. Miki, K. Yoshida, DYRK2 Is Targeted to the Nucleus and Controls p53 via Ser46 Phosphorylation in the Apoptotic Response to DNA Damage. Molecular Cell 25, 725-738 (2007).
44. R. Lara, M. J. Seckl, O. E. Pardo, The p90 RSK Family Members: Common Functions and Isoform Specificity. Cancer Research 73, 5301-5308 (2013).
45. A. Carriere, H. Ray, J. Blenis, P. P. Roux, The RSK factors of activating the Ras/MAPK signaling cascade. Front Biosci 13, 4258-4275 (2008).
46. P. P. Roux, B. A. Ballif, R. Anjum, S. P. Gygi, J. Blenis, Tumor-promoting phorbol esters and activated Ras inactivate the tuberous sclerosis tumor suppressor complex via p90 ribosomal S6 kinase. Proc Natl Acad Sci USA101, 13489-13494 (2004).
47. F. J. Sulzmaier, J. W. Ramos, RSK isoforms in cancer cell invasion and metastasis. Cancer research 73, 6099-6105 (2013).
48. H. Zhao et al., The Clinical Implications of RSK1-3 in Human Breast Cancer. Anticancer Res 36, 1267-1274 (2016).
49. K. A. Ludwik et al., Development of a RSK Inhibitor as a Novel Therapy for Triple-Negative

Breast Cancer. Mol Cancer Ther 15, 2598-2608 (2016).
50. K. A. Casalvieri, C. J. Matheson, D. S. Backos, P. Reigan, Selective Targeting of RSK Isoforms in Cancer. Trends in Cancer 3, 302-312 (2017).
51. P. A. Bignone et al., RPS6KA2, a putative tumour suppressor gene at 6q27 in sporadic epithelial ovarian cancer. Oncogene 26, 683-700 (2007).
52. A. Thakur et al., Anti-invasive and Antimetastatic Activities of Ribosomal Protein S6 Kinase 4 in Breast Cancer Cells. Clinical cancer research: an official journal of the American Association for Cancer Research 14, 4427-4436 (2008).
53. L. Lopez-Vicente et al., RSK4 inhibition results in bypass of stress-induced and oncogene-induced senescence. Carcinogenesis 32, 470-476 (2011).
54. X. Gao, D. Chaturvedi, T. B. Patel, Localization and retention of p90 ribosomal S6 kinase 1 in the nucleus: implications for its function. Mol Biol Cell 23, 503-515 (2012).
55. L. Varinou et al., Phosphorylation of the Stat1 Transactivation Domain Is Required for Full-Fledged IFN-γ-Dependent Innate Immunity. Immunity 19, 793-802 (2003).
56. P. Kovarik et al., Specificity of signaling by STAT1 depends on SH2 and C-terminal domains that regulate Ser727 phosphorylation, differentially affecting specific target gene expression. The EMBO journal 20, 91-100 (2001).
57. S. Singh, M. Kirchner, J. A. Steen, H. Steen, A practical guide to the FLEXIQuant method. Methods Mol Biol 893, 295-319 (2012).
58. N. Rappaport et al., MalaCards: an integrated compendium for diseases and their annotation. Database: the journal of biological databases and curation 2013, bat018 (2013).

TABLE 3
IFN-γ induced phospho-proteins that decreased with BI-DI1870 treatment.

| | | | | | Intensity of phospho-peptides | | | |
|---|---|---|---|---|---|---|---|---|
| | | | | | | DMSO | | BI=D1870 |
| | | | | | | DMSO | +IFNg | +IFNg |
| | | | | SEQ | | IP:RXXpS/T | IP:RXXpS/T | IP:RXXpS/T |
| | | Modifi- | | ID | DMSO | (SEQ ID | (SEQ ID | (SEQ ID |
| GENE ID | Protein | cations | Sequence | NO | IP: IgG | NO: 41) | NO: 42) | NO: 43) |
| AKAP13 | A-kinase anchor | T2471 | [R].RAETFGGFDSHQMNASK.[G] | 14 | — | 1.00 | 1.68 | 0.73 |
| | protein 13 | | | | | | | |
| AKT1S1 | Proline-rich | T266 | [R].LNTSDFQK.[L] | 15 | — | 1.00 | 3.11 | 0.89 |
| | AKT1 substrate | | | | | | | |
| | 1 | | | | | | | |
| AP1AR | AP-1 complex- | T228 | [R].SKTEEDILR.[A] | 16 | — | 1.00 | 1.67 | 0.80 |
| | associated | | | | | | | |
| | regulatory | | | | | | | |
| | protein | | | | | | | |
| CHP1 | Calcineurin B | T7 | [R].ASTLLRDEELEEIKK.[E] | 17 | — | 1.00 | 2.18 | 1.00 |
| | homologous | | | | | | | |
| | protein 1 | | | | | | | |
| CLASP1 | CLIP-associating | S646 | [R].RQSSGSATNVASTPDNR.[G] | 18 | — | 1.00 | 1.75 | 0.94 |
| | protein 1 | | | | | | | |
| CTDSPL2 | CTD small | S104 | [R].RKSQVNGEAGSYEMTNQHVK.[Q] | 19 | — | 1.00 | 8.00 | — |
| | phosphatase-like | | | | | | | |
| | protein 2 | | | | | | | |
| EHBP1L1 | EH domain- | S1257 | [R].LRRPSVNGEPGSVPPPR.[A] | 20 | — | 1.00 | 3.13 | — |
| | binding protein | | | | | | | |
| | 1-like protein 1 | | | | | | | |
| EIF4B | Eukaryotic | S422 | [R].TGSESSQTGTSTTSSR.[SN] | 21 | — | 1.00 | 2.58 | 1.29 |
| | translation | | | | | | | |
| | initiation factor | | | | | | | |
| | 4B | | | | | | | |
| FLNA | Filamin-A | S2152 | [R].RAPSVANVGSHCDLSLK.[I] | 22 | — | 1.00 | 3.67 | 1.32 |
| GPBP1 | Vasculin, | S49 | [R].RHNSSDGFDSAIGRPNGGNFGR.[K] | 23 | — | 1.00 | 2.28 | 1.00 |
| | transcription | S49 | [R].HNSSDGFDSAIGRPNGGNFGR.[K] | 24 | — | 1.00 | 2.05 | 0.45 |
| | factor | | | | | | | |
| IFNGR1 | Interferon | T295 | [R].SATLETKPESK.[Y] | 25 | — | 1.00 | 1.83 | 0.77 |
| | gamma receptor | | | | | | | |
| | 1 | | | | | | | |
| LRRC75A | Leucine-rich | T234 | [R].LTTLALNGNRLTRAVLR.[D] | 26 | — | 1.00 | 1.51 | — |
| | repeat-containing | | | | | | | |
| | protein 75A | | | | | | | |
| LUC7L3 | Luc7-like | T238 | [R].KRTEEPDRDER.[L] | 27 | — | 1.00 | 1.55 | 1.03 |
| | protein 3 | | | | | | | |
| MYO1E | Unconventional | T935 | [R].RNTTQNTGYSSGTQNANYPVR.[A] | 28 | — | 1.00 | 2.15 | 0.88 |
| | myosin-Ie | | | | | | | |
| NDRG1 | NDRG1, N-myc | S330 | [R].TASGSSVTSLDGTR.[S] | 29 | — | 1.00 | 2.29 | 1.88 |
| | downstream- | | | | | | | |
| | regulated gene 1 | | | | | | | |
| NDRG3 | NDRG3 | S338 | [R].THSTSSSLGSGESPFSR.[S] | 30 | — | 1.00 | 2.25 | 1.07 |
| REPS1 | RalBP1- | S650 | [R].RLKSEDELRPEVDEHTQK.[T] | 31 | — | 1.00 | 1.96 | 1.09 |
| | associated Eps | S650 | [R].LKSEDELRPEVDEHTQK.[T] | 32 | — | 1.00 | 1.91 | 0.94 |
| | domain- | | | | | | | |
| | containing | | | | | | | |
| | protein 1 | | | | | | | |
| RPS6 | 40S ribosomal | S235 | [K].RRRLSSLRASTSK.[S] | 33 | — | 1.00 | 1.86 | 0.74 |
| | protein S6 | S235; | [R].RLSSLRASTSK.[S] | 34 | — | 1.00 | 3.46 | 1.65 |
| | | S236 | | | | | | |
| SLC20A1 | Sodium- | S335 | [R].ERLPSVDLK.[E] | 35 | — | 1.00 | 1.51 | 0.51 |
| | dependent | | | | | | | |
| | phosphate | | | | | | | |
| | transporter 1 | | | | | | | |
| SLC4A7 | Sodium | S407 | [R].ENSTVDFSK.[VEG] | 36 | — | 1.00 | 1.79 | 1.61 |
| | bicarbonate | | | | | | | |
| | cotransporter 3 | | | | | | | |
| SPECC1L | Cytospin-A | T838 | [R].RSSTSSEPTPTVK.[T] | 37 | — | 1.00 | 2.80 | 1.80 |
| STK11IP | Serine/threonine- | S398 | [R].RASISEPSDTDPEPR.[T] | 38 | — | 1.00 | 2.31 | 0.58 |
| | protein kinase | | | | | | | |
| | 11-interacting | | | | | | | |
| | protein | | | | | | | |
| STX7 | Syntaxin-7 | S129 | [R].ASSRVSGSFPEDSSK.[E] | 39 | — | 1.00 | 1.60 | 0.67 |
| TRPM7 | Transient | S1504 | [R].RPSTEDTHEVDSK.[A] | 40 | — | 1.00 | 1.75 | 0.73 |
| | receptor | | | | | | | |
| | potential cation | | | | | | | |
| | channel | | | | | | | |
| | subfamily M | | | | | | | |
| | member 7 | | | | | | | |

1) A method of treating an inflammatory disease or disorder, the method comprising administering to a subject in need thereof an effective amount of an agent that inhibits Ribosomal S6 Kinase-1 (RSK1).

2) The method of claim 1, wherein inhibition of RSK1 is
a. inhibition of RSK1 phosphorylation;
b. inhibition of RSK1 kinase activity;
c. inhibition of the inflammatory response;
d. inhibition of phosphorylation of Signal transducer and activator of transcription 1 (STAT1);
e. inhibition of RSK1 nuclear translocation;
f. inhibition of RSK1 expression level and/or activity; and/or
g. suppression of IFN-γ-induced pro-inflammatory chemokines in primary macrophages.

3) The method of claim 2, wherein the RSK1 phosphorylation is at Serine 380.

4)-6) (canceled)

7) The method of claim 2, wherein the phosphorylation of STAT1 is at Serine 727.

8) (canceled)

9) The method of claim 1, further comprising, prior to administration,
a. diagnosing a subject with having an inflammatory disease or disorder; or
b. receiving results that identify a subject as having an inflammatory disease or disorder.

10) (canceled)

11) The method of claim 1, wherein the agent that inhibits RSK1 is selected from the group consisting of a small molecule, an antibody, a peptide, a genome editing system, an antisense oligonucleotide, and an RNAi.

12) The method of claim 11, wherein the small molecule is selected from the group consisting of: MK-1775, Manumycin-a, Cerulenin, Tanespimycin, salermide, and tosedostat.

13) The method of claim 11, wherein the RNAi is a microRNA, an siRNA, or a shRNA.

14) The method of claim 11, wherein the antibody is a humanized antibody.

15) (canceled)

16) The method of claim 2, wherein the expression level and/or activity of RSK1 is inhibited by at least 50%, at least 60%, at least 70%, at least 80%, at least 90%, or more as compared to an appropriate control.

17) (canceled)

18) The method of claim 2, wherein the IFN-γ-induced chemokines are suppressed by at least 50%, at least 60%, at least 70%, at least 80%, at least 90%, or more as compared to an appropriate control.

19) The method of claim 1, further comprising administering at least a second therapeutic for an inflammatory disease or disorder.

20) A method of treating an inflammatory disease or disorder, the method comprising administering to a subject in need thereof an effective amount of an agent that inhibits Signal transducer and activator of transcription 1 (STAT1) phosphorylation.

21) The method of claim 20, wherein STAT1 phosphorylation is at Serine 727.

22) The method of claim 20, wherein inhibition of STAT1 phosphorylation inhibits the inflammatory response.

23) The method of claim 20, further comprising, prior to administration,
a. diagnosing a subject with having an inflammatory disease or disorder; or
b. receiving results that identify a subject as having an inflammatory disease or disorder.

24) (canceled)

25) The method of claim 20, wherein the agent that inhibits STAT1 phosphorylation is selected from the group consisting of a small molecule, an antibody, a peptide, a genome editing system, an antisense oligonucleotide, and an RNAi.

26)-31) (canceled)

32) The method of claim 1, wherein the inflammatory disease or disorder is selected from the group consisting of: macrophage activation syndrome, ulcerative colitis, type II diabetes, rheumatoid arthritis, juvenile idiopathic arthritis, Takayasu disease, aortic stenosis, Coffin-Lowry syndrome, pulmonary hypertension, Gaucher disease, systemic lupus erythematosus, Buerger disease, atherosclerosis, coronary artery disease, myocardial infarction, peripheral artery disease, vein graft disease, in-stent restenosis, arterioveneous fistula disease, arterial calcification, calcific aortic valve disease, Crohn's disease, vasculitis syndrome, scleroderma, rheumatic heart disease, acute lung injury, chronic obstructive pulmonary disease, acute kidney injury, stroke, neuroinflammation, and fatty liver.

33) (canceled)

34) (canceled)

35) A composition comprising an agent that inhibits RSK1 or an agent that inhibits STAT1 phosphorylation.

36) (canceled)

37) The composition of claim 35, further comprising a pharmaceutically acceptable carrier.
